SEQUENCE LISTING (51 sequences)

SEQ ID NO: 1 - DNA, 387 bp, Artificial Sequence
  Synthetic nucleic acid
caggtgcggctggtgcaatctgggggaggcctggtcaagcctggggggtccctgagactctcctgtgcagcctctggattcaccttcagcacctttgccatgcattgggtccgccaggctccagggaaggggctggagtgggtctcatccattagtcgtagtagtggttccataaactacgcagactcagtgaagggccgattcaccatctccagagacaacgccaagaactcactgtttctgcaaatgaacagcctgagagccgacgacacggctgtctattactgtgcgcgagagaacacgattccgtttgggggaggtgtcgtccttgaaagggcatcacactttgactactggggccagggaaccacggtcaccgtctcttca

SEQ ID NO: 2 - protein, 129 aa, Artificial Sequence
  Synthetic polypeptide
QVRLVQSGGGLVKPGGSLRLSCAASGFTFSTFAMHWVRQAPGKGLEWVSSISRSSGSINYADSVKGRFTISRDNAKNSLFLQMNSLRADDTAVYYCARENTIPFGGGVVLERASHFDYWGQGTTVTVSS

SEQ ID NO: 3 - DNA, 321 bp, Artificial Sequence
  Synthetic nucleic acid
gacatccgggtgacccagtctccatcctccctgtctgcatctgtaggagacagagtcaccatcatttgccgggcaagtcagagcagtagtactttcctaaattggtatcagcagaaaccagggaaagcccctaacctcctgatctacgctgcatccagtttgcaaagtggggtcccatcaaggttcagtggcagtggatctgggacagatttcactctcaccatcagcagtctgcaaccagaagattttgcaacttactactgtcaacagagttacagtgccccgtacacttttggccaggggaccaaagtggatatcaaa

SEQ ID NO: 4 - protein, 107 aa, Artificial Sequence
  Synthetic polypeptide
DIRVTQSPSSLSASVGDRVTIICRASQSSSTFLNWYQQKPGKAPNLLIYAASSLQSGVPSRFSGSGSGTDFTLTISSLQPEDFATYYCQQSYSAPYTFGQGTKVDIK

SEQ ID NO: 5 - DNA, 369 bp, Artificial Sequence
  Synthetic nucleic acid
caggtgcagctggtgcagtctggagcagaggtgaaaaagcctgggacctcagtgaaaatttcctgcaaggcatctggatacagcttcaccagcaagtatatgcactgggtgcgacaggcccctggacaagggcttgagtggatgggaaaaatcaaccctagtggtggtagaagagactacgcacagaagttccagggcagagtcaccatgaccagggacacgtccacgagcacagtctacatggagctgagcagcctgagatctggggacacggccgtctattactgtgcgagagatatgcacggtgtgttaagctggtaccatgcccttgactactggggccagggaaccctggtcaccgtctcctca

SEQ ID NO: 6 - protein, 123 aa, Artificial Sequence
  Synthetic polypeptide
QVQLVQSGAEVKKPGTSVKISCKASGYSFTSKYMHWVRQAPGQGLEWMGKINPSGGRRDYAQKFQGRVTMTRDTSTSTVYMELSSLRSGDTAVYYCARDMHGVLSWYHALDYWGQGTLVTVSS

SEQ ID NO: 7 - DNA, 321 bp, Artificial Sequence
  Synthetic nucleic acid
gacatccgggtgacccagtctccatcctccctgtctacgtctgtgggagacagagtcaccatcacttgccgggcaagtcaagacattggtagatatctaaattggtatcagcagaaaccagggaaagcccctaagctcctgatctatggtgcatccagtttgcaaagtggggtcccatcaaggttcagtggcagtggatctgggacagatttcactctcaccatcagcagtctgcaacctgaagattttgcaacttactactgtcaacagggttaccgcatcccgatcaccttcggccaagggacacgactggagattaaa

SEQ ID NO: 8 - protein, 107 aa, Artificial Sequence
  Synthetic polypeptide
DIRVTQSPSSLSTSVGDRVTITCRASQDIGRYLNWYQQKPGKAPKLLIYGASSLQSGVPSRFSGSGSGTDFTLTISSLQPEDFATYYCQQGYRIPITFGQGTRLEIK

SEQ ID NO: 9 - DNA, 378 bp, Artificial Sequence
  Synthetic nucleic acid
caggtgcagctgcaggagtcgggcccaggactagtgaagccttcggagaccctgtccctcacctgcactgtctctggtgactccctcagtagttcgtactggagctggatccggcagtccgccgggaagggactggagtacattgggcgtacctatgttagtgggaacaccaagtacaacccctccctcaagagtcgagtcaccatgtcagtagacacgtccaagaaccagttctccctgagactgacctctgtgaccgccgcggacacggccgtatattactgtgcgagaatacgagtgctaccagctgctatgcttagaggggactactggtacttcgatctctggggccgtggcaccctggtcactgtctcctca

SEQ ID NO: 10 - protein, 126 aa, Artificial Sequence
  Synthetic polypeptide
QVQLQESGPGLVKPSETLSLTCTVSGDSLSSSYWSWIRQSAGKGLEYIGRTYVSGNTKYNPSLKSRVTMSVDTSKNQFSLRLTSVTAADTAVYYCARIRVLPAAMLRGDYWYFDLWGRGTLVTVSS

SEQ ID NO: 11 - DNA, 318 bp, Artificial Sequence
  Synthetic nucleic acid
tcctatgagctgacgcagctaccctcagtgtccgtgtcaccaggacagacagcgagcatcacctgctctggagataaagtggaaaataaatatgtttgctggtatcagcagaagtcaggccagtcccctgtcctggtcatctatgaagatagtaagcggccctcagggatccctgagcgattctctggctccaactctggaaacacagccactctgaccatcagcgggacccaaactatggatgaggctgactatttctgtcaggcgtgggacagtagtattggggtcttcggaactgggaccaagctcaccgtccta

SEQ ID NO: 12 - protein, 106 aa, Artificial Sequence
  Synthetic polypeptide
SYELTQLPSVSVSPGQTASITCSGDKVENKYVCWYQQKSGQSPVLVIYEDSKRPSGIPERFSGSNSGNTATLTISGTQTMDEADYFCQAWDSSIGVFGTGTKLTVL

SEQ ID NO: 13 - DNA, 369 bp, Artificial Sequence
  Synthetic nucleic acid
caggtgcagctggtggagtctgggggaggcttggtaaagcctggggggtcccttagactctcctgtgcagcctctggattcatgttcagtaatgcctggatgaactgggtccgccaggctccagggaaggggctggagtgggttggacgtattaaaagcagaagtgatggtgggacaacagactacgctgcacccgtgaaaggcagattcaccttctcaagagaggattcaaaaaacatgctgtatctgcaaatgaacagcctgaaacgcgaggacacagccgtctattactgtaccaccagagtttctatttttcggggacctattgaggacgtctggggccaagggaccacggtcaccgtctcctca

SEQ ID NO: 14 - protein, 123 aa, Artificial Sequence
  Synthetic polypeptide
QVQLVESGGGLVKPGGSLRLSCAASGFMFSNAWMNWVRQAPGKGLEWVGRIKSRSDGGTTDYAAPVKGRFTFSREDSKNMLYLQMNSLKREDTAVYYCTTRVSIFRGPIEDVWGQGTTVTVSS

SEQ ID NO: 15 - DNA, 336 bp, Artificial Sequence
  Synthetic nucleic acid
cagcctgtgctgactcagccgccctcagtgtctggggccccagggcagagggtcaccatctcctgcgctggaagcagctccaacatcggggcaggttatgatgtatactggtaccagcagcttccaggaactgcccccaaactcctcatctatggaaacaacaatcggccctcaggggtccctgaccgattctctggctccaagtctggcacctcagcctccctggccatcacagggctccaggctgaggatgaggctgaatattactgccagtcctatgacagcagcctgcgtgattcttatgtcttcggaagtgggaccaaggtgaccgtccta

SEQ ID NO: 16 - protein, 112 aa, Artificial Sequence
  Synthetic polypeptide
QPVLTQPPSVSGAPGQRVTISCAGSSSNIGAGYDVYWYQQLPGTAPKLLIYGNNNRPSGVPDRFSGSKSGTSASLAITGLQAEDEAEYYCQSYDSSLRDSYVFGSGTKVTVL

SEQ ID NO: 17 - DNA, 408 bp, Artificial Sequence
  Synthetic nucleic acid
gaggtccagctggtggagtctgggggaggcctggtcaagcctggggggtccctgagactctcctgtgcagcctctggatttaccctcagtagttatagcatgaactgggtccgccaggctccagggaaggggctggaatgggtctcatccgttactagtagtgatgacaaatactacgcaggccgatttaccctctctacaggcagcagtgatgataaatactacgcagactcagtgaggggccgctttaccatctccagagacaacgccaagaattcactctatctgcaaatgaacagcctgagagccgaagacacagctatatattattgtgcgagggatattggatgggcacaaccgcctggggctgactactggggccagggaaccctggtcaccgtctcctca

SEQ ID NO: 18 - protein, 137 aa, Artificial Sequence
  Synthetic polypeptide
EVQLVESGGGLVKPGGSLRLSCAASGFTLSSYSMNWVRQAPGKGLEWVSSVTSSDDKYYAGRFTLSTGSSDDKYYADSVRGRFTISRDNAKNSLYLQMNSLRAEDTAIYYCARDIGWAQPPGADYWGQGTLVTVSSH

SEQ ID NO: 19 - DNA, 321 bp, Artificial Sequence
  Synthetic nucleic acid
gacatccagttgacccagtctccatcctccctgtctgcatctgtaggagacagagtcaccatcacttgccgggccagtcagggcattagaagttatttagcctggtatcagcaaaaaccagggaaagcccctaagctcctgatctatgctgcatccactttgcaaagtggggtcccatcaaggttcagcggcagtggatctgggacagatttcactctcaccatcagcagcctgcagcctgaagattttgcaacttattactgtcaacaggttaatagttaccctcggactttcggccaagggaccaaggtggaaatcaaa

SEQ ID NO: 20 - protein, 107 aa, Artificial Sequence
  Synthetic polypeptide
DIQLTQSPSSLSASVGDRVTITCRASQGIRSYLAWYQQKPGKAPKLLIYAASTLQSGVPSRFSGSGSGTDFTLTISSLQPEDFATYYCQQVNSYPRTFGQGTKVEIK

SEQ ID NO: 21 - DNA, 396 bp, Artificial Sequence
  Synthetic nucleic acid
cagctgcagctgcaggagtcgggcccaggactgctgaagccttcggagaccctgtccctcacttgcagtgtctctggtggctccatcaacagttatacttactactggggctgggtccgccagtccccagcgaaggggctggagtggattgggagtttctcttatagtgggagttcccactacaacccgtctcttgagagtcgagtcaccatctccgtagacaggtccaagaatcaggtctccctgaagctgagttctgtgaccgccgcagacacggctgtgtattactgtgcgagattcgctaggtttatgactacgtctggggatcttatcgttagtctcgattactacgctttcgacgtctggggccaagggaccacggtcaccgtctcctca

SEQ ID NO: 22 - protein, 132 aa, Artificial Sequence
  Synthetic polypeptide
QLQLQESGPGLLKPSETLSLTCSVSGGSINSYTYYWGWVRQSPAKGLEWIGSFSYSGSSHYNPSLESRVTISVDRSKNQVSLKLSSVTAADTAVYYCARFARFMTTSGDLIVSLDYYAFDVWGQGTTVTVSS

SEQ ID NO: 23 - DNA, 324 bp, Artificial Sequence
  Synthetic nucleic acid
gacatccagatgacccagtctccatcctccctgtctgcatctgtaggagacagagtcaccatcacttgccgggcaagtcagaccattaggaacaatttaaattggtatcagcaaaaactagggaaagcccctaaactcctgatctatgctgcatccactttacaaaatggggtcccctcgaggttcagtggcagtggatctgggacagatttcactctcaccatcagtagtctgcaacctgaagattttgcgacttactattgtcaacagagttacactacccctcgagtcacttttgcccaggggaccaagttggagatcaaa

SEQ ID NO: 24 - protein, 108 aa, Artificial Sequence
  Synthetic polypeptide
DIQMTQSPSSLSASVGDRVTITCRASQTIRNNLNWYQQKLGKAPKLLIYAASTLQNGVPSRFSGSGSGTDFTLTISSLQPEDFATYYCQQSYTTPRVTFAQGTKLEIK

SEQ ID NO: 25 - DNA, 357 bp, Artificial Sequence
  Synthetic nucleic acid
gaggtccagctggtggagtctgggggaggcttggtgaagcctggggggtcccttagactctcctgtgcaggctctggattcactttcactaaagcctggatgagctgggtccgccaggctccagggaaggggctggagtgggttggccgtattaagagcagagctgatagtgggacaacagcctacactgcacccgtgaaaggcagattcaccatctcaagagatgattcaaaaaagacgctgtatctgcaaatgaacagcctgaaaaccgaggacacagccgtgtactattgtgtcgcacatggggacccagtagaggcacaatggggccagggaaccctggtcaccgtctcctct

SEQ ID NO: 26 - protein, 119 aa, Artificial Sequence
  Synthetic polypeptide
EVQLVESGGGLVKPGGSLRLSCAGSGFTFTKAWMSWVRQAPGKGLEWVGRIKSRADSGTTAYTAPVKGRFTISRDDSKKTLYLQMNSLKTEDTAVYYCVAHGDPVEAQWGQGTLVTVSS

SEQ ID NO: 27 - DNA, 336 bp, Artificial Sequence
  Synthetic nucleic acid
cagtctgtgctgactcagccgccctcagtgtctggggccccagggcagagggtcaccatctcctgcactgggggcagctccaacatcggggcaggttatgatgtacaatggtaccagcaggttccaggaacagcccccaaactcctcatctatcataacaacaatcggccctcaggggtccctgaccggttctctggctccaagtctggcacctcagcctccctggccatcactgggctccaggctgaggatgaggctgattattactgccagtcttatgacagcagcctgagtgacaattgggtgttcggcggagggaccaagctgaccgtccta

SEQ ID NO: 28 - protein, 112 aa, Artificial Sequence
  Synthetic polypeptide
QSVLTQPPSVSGAPGQRVTISCTGGSSNIGAGYDVQWYQQVPGTAPKLLIYHNNNRPSGVPDRFSGSKSGTSASLAITGLQAEDEADYYCQSYDSSLSDNWVFGGGTKLTVL

SEQ ID NO: 29 - DNA, 375 bp, Artificial Sequence
  Synthetic nucleic acid
caggtccagctggtgcagtctggggctgaggtgaagaagcctgggacctcagtgagggtctcctgcaaggcttctggatacagcctcaccggccactatatgcactgggtgcgacaggcccctggacaagggcttgagtggatgggttggatcaaccctgccagtggtggcacacattatgcagagaagtttcgggtcagggtcgccatgaccagggacacgtccatcagcacagtttacatggagttgtacagcctgacatctgacgacacggccgtctactactgtgcgagggctgtccggggcacgacagcagtggctgggacttggaggttcgacccctggggccagggaaccctggtcatcgtttcctca

SEQ ID NO: 30 - protein, 125 aa, Artificial Sequence
  Synthetic polypeptide
QVQLVQSGAEVKKPGTSVRVSCKASGYSLTGHYMHWVRQAPGQGLEWMGWINPASGGTHYAEKFRVRVAMTRDTSISTVYMELYSLTSDDTAVYYCARAVRGTTAVAGTWRFDPWGQGTLVIVSS

SEQ ID NO: 31 - DNA, 330 bp, Artificial Sequence
  Synthetic nucleic acid
cagtctgtgctgactcagccgccctcagtgtctgcggccccaggacagaaggtcaccatctcctgctctggaagcagctccaacattgggaataattatgtatcctggtaccagcagttcccaggtacagcccccaaactcctcatttatgacaataataggcgaccctcaggtgttcctgaccgattctctggctccaagtctgacacgtcagccaccctgggcatcaccggactccagactggggacgaggccgattattactgcggaacatgggatagcagcctgggtgctggtgtcttcggcggagggaccaagctgaccgtcctg

SEQ ID NO: 32 - protein, 110 aa, Artificial Sequence
  Synthetic polypeptide
QSVLTQPPSVSAAPGQKVTISCSGSSSNIGNNYVSWYQQFPGTAPKLLIYDNNRRPSGVPDRFSGSKSDTSATLGITGLQTGDEADYYCGTWDSSLGAGVFGGGTKLTVL

SEQ ID NO: 33 - DNA, 32 bp, Artificial Sequence
  Synthetic nucleic acid
tgccatgggaaccagcagctagcgttttagca

SEQ ID NO: 34 - DNA, 45 bp, Artificial Sequence
  Synthetic nucleic acid
gttctaggcgcgcctgtacttgctgaggagacrgtgaccagggtg

SEQ ID NO: 35 - DNA, 44 bp, Artificial Sequence
  Synthetic nucleic acid
gttctaggcgcgcctgtacttgctgaagagacggtgaccattgt

SEQ ID NO: 36 - DNA, 44 bp, Artificial Sequence
  Synthetic nucleic acid
gttctaggcgcgcctgtacttgctgaggagacggtgaccagggt

SEQ ID NO: 37 - DNA, 46 bp, Artificial Sequence
  Synthetic nucleic acid
gttctaggcgcgcctgtacttgctgaggagacggtgaccgtggtcc

SEQ ID NO: 38 - DNA, 40 bp, Artificial Sequence
  Synthetic nucleic acid
cttatagcggccgcagttcgtttgatttccaccttggtcc

SEQ ID NO: 39 - DNA, 40 bp, Artificial Sequence
  Synthetic nucleic acid
cttatagcggccgcagttcgtttgatctccascttggtcc

SEQ ID NO: 40 - DNA, 40 bp, Artificial Sequence
  Synthetic nucleic acid
cttatagcggccgcagttcgtttgatatccactttggtcc

SEQ ID NO: 41 - DNA, 40 bp, Artificial Sequence
  Synthetic nucleic acid
cttatagcggccgcagttcgtttaatctccagtcgtgtcc

SEQ ID NO: 42 - DNA, 43 bp, Artificial Sequence
  Synthetic nucleic acid
cttatagcggccgcgggctgacctaggacggtsascttggtcc

SEQ ID NO: 43 - DNA, 43 bp, Artificial Sequence
  Synthetic nucleic acid
cttatagcggccgcgggctgacctaaaatgatcagctgggttc

SEQ ID NO: 44 - DNA, 43 bp, Artificial Sequence
  Synthetic nucleic acid
cttatagcggccgcgggctgacctaggacggtcagctcsgtcc

SEQ ID NO: 45 - DNA, 43 bp, Artificial Sequence
  Synthetic nucleic acid
cttatagcggccgcgggctgaccgaggacggtcacttggtcca

SEQ ID NO: 46 - DNA, 43 bp, Artificial Sequence
  Synthetic nucleic acid
cttatagcggccgcgggctgaccgaggrcggtcagctgggtgc

SEQ ID NO: 47 - DNA, 21 bp, Artificial Sequence
  Synthetic nucleic acid
ggaagtagtccttgaccaggc

SEQ ID NO: 48 - DNA, 25 bp, Artificial Sequence
  Synthetic nucleic acid
ctctctgggatagaagttattcagc

SEQ ID NO: 49 - DNA, 23 bp, Artificial Sequence
  Synthetic nucleic acid
ccagggtagctttgttcgcttgc

SEQ ID NO: 50 - DNA, 60 bp, Artificial Sequence
  Synthetic nucleic acid
  misc_feature (34)..(37): n is a, c, g, or t
tctcgtgggctcggagatgtgtataagagacagnnnnctgttattgctagcgttttagca

SEQ ID NO: 51 - DNA, 56 bp, Artificial Sequence
  Synthetic nucleic acid
  misc_feature (34)..(37): n is a, c, g, or t
tcgtcggcagcgtcagatgtgtataagagacagnnnnaaggcgcgcctgtacttgc

(19) United States
(12) Patent Application Publication
(10) Pub. No.: US 2022/0089694 A1
(43) Pub. Date: Mar. 24, 2022

(54) EBOLA VIRUS GLYCOPROTEIN-SPECIFIC MONOCLONAL ANTIBODIES AND USES THEREOF

(71) Applicant: The U.S.A., as represented by the Secretary, Department of Health and Human Services, Bethesda, MD (US)

(72) Inventors: Nancy J. Sullivan, Kensington, MD (US); John Misasi, Kensington, MD (US); Brandon Dekosky, Lawrence, KS (US); Kendra Elizabeth Leigh, Frankfurt (DE)

(73) Assignee: The U.S.A., as represented by the Secretary, Department of Health and Human Services, Bethesda, MD (US)

(21) Appl. No.: 17/415,412

(22) PCT Filed: Dec. 19, 2019
(86) PCT No.: PCT/US2019/067423

§ 371 Date: Jun. 17, 2021

Related U.S. Application Data
(60) Provisional application No. 62/782,809, filed on Dec. 20, 2018.

(51) Int. Cl. C07K 16/10; G01N 33/569

(52) U.S. Cl. CPC C07K 16/10; G01N 33/56983; C07K 2317/24; G01N 2800/26; C07K 2317/92; G01N 2333/08; C07K 2317/76

(57) ABSTRACT

Human monoclonal antibodies that specifically bind Ebola virus glycoprotein with nanomolar affinity are described. The monoclonal antibodies were isolated by bulk sorting of plasmablasts from a human Ebola virus vaccinee and pairing of the immunoglobulin heavy and light chain genes using emulsion PCR. The paired immunoglobulin genes were expressed using Fab yeast display to screen and characterize the antibodies. The Ebola virus-specific monoclonal antibodies can be used, for example, to diagnose and treat Ebola virus infection or Ebola virus disease in a subject.

### CROSS REFERENCE TO RELATED APPLICATIONS

This application claims the benefit of U.S. Provisional Application No. 62/782,809, filed Dec. 20, 2018, which is herein incorporated by reference in its entirety.

### FIELD

This disclosure concerns monoclonal antibodies and antigen binding fragments that specifically bind to Ebola virus (EBOV) glycoprotein (GP) and their use, for example, in methods of treating or preventing EBOV infection or EBOV disease (EVD) in a subject.

### BACKGROUND

In 2013, the International Committee on the Taxonomy of Viruses (ICTV) Filoviridae Study Group and other experts published an updated taxonomy for filoviruses. The genus Ebolavirus is one of three genera in the family Filoviridae, which along with the genera Marburgvirus and Cuevavirus, are known to induce viral hemorrhagic fever. Five distinct species included in the genus Ebolavirus are Bundibugyo (BDBV), Reston (RESTV), Sudan (SUDV), Taï Forest (TAFV), and Zaire (EBOV) (Kuhn et al., Arch Virol 158(1): 301-311, 2013).

Ebola virus is a large, negative-strand RNA virus composed of seven genes encoding viral proteins, including a single glycoprotein (GP) (Sanchez et al., Virus Res 29(3): 215-240, 1993; Sanchez et al., J Gen Virol 73(Pt 2): 347-357, 1992; Hart, Int J Parasitol 33(5-6): 583-595, 2003). The virus is responsible for causing Ebola virus disease (EVD), formerly known as Ebola hemorrhagic fever (EHF), in humans. In particular, BDBV, EBOV, and SUDV have been associated with large outbreaks of EVD in Africa with reported case fatality rates of up to 90% (Baize et al., N Engl J Med 371(15): 1418-1425 2014). Transmission of Ebola virus to humans is not yet fully understood, but is likely due to incidental exposure to infected animals (Geisbert and Jahrling, Nat Med 10(12 Suppl): S110-S121, 2004; Meslin, Emerg Infect Dis 3(2):223-228, 1997; Okware et al., Trop Med Int Health 7(12): 1068-1075, 2002). EVD spreads through human-to-human transmission, with infection resulting from direct contact with blood, secretions, organs or other bodily fluids of infected people, and indirect contact with environments contaminated by such fluids (Baize et al., N Engl J Med 371(15): 1418-1425 2014).

EVD has an incubation period of 2 to 21 days (7 days on average, depending on the strain) followed by a rapid onset of non-specific symptoms such as fever, extreme fatigue, gastrointestinal complaints, abdominal pain, anorexia, headache, myalgias and/or arthralgias. These initial symptoms last for about 2 to 7 days after which more severe symptoms related to hemorrhagic fever occur, including hemorrhagic rash, epistaxis, mucosal bleeding, hematuria, hemoptysis, hematemesis, melena, conjunctival hemorrhage, tachypnea, confusion, somnolence, and hearing loss. Laboratory findings include low white blood cell and platelet counts and elevated liver enzymes (Baize et al., N Engl J Med 371(15): 1418-1425 2014). In general, the symptoms last for about 7 to 14 days after which recovery may occur. Death can occur 6 to 16 days after the onset of symptoms (Geisbert and Jahrling, Nat Med 10(12 Suppl): S110-S121, 2004; Hensley et al., Curr Mol Med 5(8): 761-772, 2005). People are infectious as long as their blood and secretions contain the virus; the virus was isolated from semen 61 days after onset of illness in a man who was infected in a laboratory (Baize et al., N Engl J Med 371(15): 1418-1425 2014).

Immunoglobulin M (IgM) antibodies to the virus appear 2 to 9 days after infection, whereas immunoglobulin G (IgG) antibodies appear approximately 17 to 25 days after infection, which coincides with the recovery phase. In survivors of EVD, both humoral and cellular immunity are detected, however, their relative contribution to protection is unknown (Sullivan et al., J Virol 77(18): 9733-9737, 2003).

While prior outbreaks of EVD have been localized to regions of Africa, there is a potential threat of spread to other countries given the frequency of international travel. The 2014 outbreak in West Africa was first recognized in March 2014, and by Apr. 13, 2016, the number of cases far exceeded the largest prior EVD outbreak with a combined total (suspected, probable, and laboratory-confirmed) 28,616 cases and 11,310 deaths (case fatality rate=39.5%) (Centers for Disease Control and Prevention, 2014 Ebola Outbreak in West Africa—Case Counts, Apr. 13, 2016). The largest previous outbreak occurred in Uganda in 2000-2001 with 425 cases and 224 deaths (case-fatality rate=53%) (Dixon and Schafer, Morbidity and Mortality Weekly Report 63:1-4, 2014).

Viruses in the Filoviridae family are also categorized as potential threats for use as biological weapons due to ease of dissemination and transmission, and high levels of mortality. Currently, no effective therapies or FDA-licensed vaccines exist for any member of the Filoviridae family of viruses.

### SUMMARY

The present disclosure describes eight human Ebola virus (EBOV) glycoprotein (GP)-specific monoclonal antibodies that were isolated from plasmablasts of a human EBOV vaccine recipient. The antibodies are referred to herein as EboV.YD.01, EboV.YD.02, EboV.YD.03, EboV.YD.04, EboV.YD.05, EboV.YD.06, EboV.YD.07, and EboV.YD.08. The disclosed antibodies can be used, for example, in the treatment or diagnosis of EBOV infection or EVD.

Provided herein are monoclonal antibodies (or antigen-binding fragments) that bind, such as specifically bind, EBOV GP. In some embodiments, the monoclonal antibody or antigen-binding fragment includes the complementarity determining region (CDR) sequences of EboV.YD.01, EboV.YD.02, EboV.YD.03, EboV.YD.04, EboV.YD.05, EboV.YD.06, EboV.YD.07, or EboV.YD.08.

Also provided are nucleic acid molecules and expression vectors that encode the VH and/or VL domain of a monoclonal antibody disclosed herein.

Further provided are pharmaceutical compositions for treating or inhibiting an Ebola virus infection, which include a therapeutically effective amount of a monoclonal antibody, antigen-binding fragment, nucleic acid molecule, or expression vector disclosed herein and a pharmaceutically acceptable carrier.

Also provided are methods of detecting an EBOV infection in a subject by contacting a biological sample from the subject with a monoclonal antibody or antigen binding fragment disclosed herein under conditions sufficient to form an immune complex; and detecting the presence of the immune complex in the sample.

Further provided are methods of preventing or treating an EBOV infection in a subject by administering to the subject a therapeutically effective amount of a monoclonal antibody, antigen binding fragment, nucleic acid molecule, expression vector, or pharmaceutical composition disclosed herein.

A method of producing a monoclonal antibody disclosed herein is further provided. The method includes expressing first and second nucleic acid molecules encoding the VH domain and the VL domain, respectively, of a monoclonal antibody or antigen binding fragment disclosed herein in a host cell; or expressing a nucleic acid molecule encoding the VH domain and the VL domain of a monoclonal antibody or antigen binding fragment disclosed herein in the host cell; and purifying the antibody or antigen binding fragment.

The foregoing and other objects, features, and advantages of the disclosure will become more apparent from the following detailed description, which proceeds with reference to the accompanying figures.

### BRIEF DESCRIPTION OF THE DRAWINGS

FIGS. 1A-1D: High-throughput cloning, yeast display, and functional analysis of the human natively paired VH:VL antibody repertoire. (FIG. 1A) Using an axisymmetric flow focusing device, peripheral blood B cells were compartmentalized inside microdroplets and lysed, and single-cell mRNA was captured as overlap extension PCR template to produce VH and VL cDNAs joined by a 32-nucleotide linker sequence (SEQ ID NO: 33) containing NcoI and NheI restriction sites. (FIG. 1B) Natively paired VH:VL amplicon libraries were subcloned en masse into a Fab expression vector with a galactose-inducible bidirectional promoter Gal1/Gal10) for transcription of CH1-VH and C_{κ/λ}-V_{κ/λ}, along with expression tags (c-Myc and FLAG, respectively) and leucine-zipper (LZ) dimerization domains. (FIG. 1C) Display characteristics for a panel of 13 anti-hemagglutinin (HA) antibodies in yeast strains EBY100, AWY101 that overexpresses protein disulfide isomerase (PDI), and AWY101 with LZ-forced dimerization. (FIG. 1D) Sequential rounds of FACS under increasingly stringent conditions (such as lower antigen concentrations, co-incubation with competitor antibodies) were used to bin libraries within various windows of affinity. Antibodies were recovered from sorted yeast, and expressed and characterized.

FIGS. 2A-2C: Examples of natively paired antibody repertoire analysis and functional characterization. (FIG. 2A) FACS analysis of the natively paired heavy:light antibody repertoire from 5,002 peripheral plasmablasts isolated from an EBOV vaccinee and screened for binding to EBOV GP_{Δmuc}. (FIG. 2B) Neutralization and affinity of GP_{Δmuc} antibodies randomly selected after the third round of sorting. Affinity values and neutralization are reported as average±s.d. from three technical replicates. (FIG. 2C) Competition analysis of anti-GP_{Δmuc} antibodies from FIG. 2B and the EBOV-neutralizing antibody KZ52.

FIGS. 3A-3B: Maps of pCT-VHVL-K1 (FIG. 3A) and pCT-VHVL-L1 (FIG. 3B) native VH:VL display vectors. Natively-paired VH:VL sequences were cloned en masse into these vectors for human antibody repertoire mining, and their corresponding Fabs were expressed on the yeast cell surface via galactose induction.

FIGS. 4A-4B: Flow cytometry analysis of a panel of human anti-HA antibodies before and after display optimization, and of the 2 anti-EBOV antibodies and 1 anti-HIV-1 bNAb in the optimized system. (FIG. 4A) Display of the six anti-HA antibodies listed in FIG. 1C that did not functionally display in EBY100 (upper) and in AWY101 with LZ-forced Fab dimerization (lower). (FIG. 4B) Anti-EBOV antibodies c13c6 and KZ52 and anti-HIV-1 bNAb VRC34.01 displayed in the optimized system. For anti-HA antibodies, 100 nM recombinant A/California/07/2009 HA was used to stain D1 H1-2, D1 H1-3/H3-3, D1 H1-53, D1 H1-12, and D1 H1-17/H3-14, and 100 nM recombinant B/Brisbane/60/2008 HA was used to stain D1 Vic-8/Yama-20. 23 nM GP_{Δmuc}-APC was used to stain c13c6 and KZ52; 50 nM VRC34-epitope scaffold-FP-APC was used to stain VRC34.01. A representative profile from five (FIG. 4A) or three (FIG. 4B) independent experiments for each antibody is shown.

FIG. 5: Representative FACS gating strategy for EBOV library sorts. Yeast cells were stained with 2 μg/ml anti-FLAG-FITC and 23 nM GP_{Δmuc}-APC.

FIG. 6: Flow cytometry antigen binding profiles of monoclonal yeast populations expressing EBOV.YD.09-EBOV.YD.11, which were identified by single colony picking. Yeast cells were stained with 2 μg/ml anti-FLAG-FITC and 23 nM GP GP_{Δmuc}-APC.

FIGS. 7A-7B: Biolayer interferometry response curves for human anti-EBOV antibodies from the plasmablast cognate VH:VL repertoire. Binding was assessed against GP_{Δmuc}. Global analyses were carried out using nonlinear least-squares fitting allowing a single set of binding parameters to be obtained simultaneously for all concentrations used in each experiment. Shown are EBOV.YD.01-EBOV.YD.04 (FIG. 7A) and EBOV.YD.05-EBOV.YD.08 (FIG. 7B).

FIG. 8: Neutralization of EBOV GP pseudotype infection by human anti-EBOV antibodies. Percent (%) infection is shown relative to the negative control antibody VRC01. Data are reported as average±standard deviation for three technical replicates.

FIGS. 9A-9D: Yeast display enrichment and selection of Ebola GP-specific antibodies. Yeast display libraries with kappa light chains (EboV K) and lambda light chains (EboV L) were sequentially enriched for binding to the Ebola GPΔMuc probe labeled with allophycocyanin (APC). (FIGS. 9A, 9C) Flow scatter plots of library binding in Rounds 1-3 showing enriched binding in the APC positive gate in successive rounds. (FIGS. 9B, 9D) Flow scatter plot showing Fab expression (Flag-VL FITC) vs. GPΔMuc-APC positive yeast. Sorting gates used to identify candidate mAbs are shown and were set to correspond with presumed increases in affinity.

FIGS. 10A-10B: Binding of EboV.YD.01 to GPΔMuc. (FIG. 10A) Yeast from probe positive sort gates was diluted and a single colony was purified by limiting dilutions. The colony was grown in culture and binding to GPΔMuc-APC probe was confirmed using flow cytometry. (FIG. 10B) The sequence of the expressed antibody in the yeast from FIG. 10A was obtained, cloned into full immunoglobulin G (IgG) expression vectors, and the antibody was expressed and purified. Biolayer interferometry was used to assess binding of the purified IgG to GPΔMuc. GPΔMuc was coupled to amine-reactive 2nd generation biosensors and binding of the indicated monoclonal antibodies was determined. EboV kappa low was derived from yeast expected to have low binding capacity. KZ52 is an Ebola-specific positive control mAb and VRC01 is a HIV gp120-directed negative control mAb. Shown are representative binding curves from 13 replicates.

FIG. 11: Competition group as determined by BLI. The order of addition to the biosensors was mucin-domain-deleted GP (antigen), competitor mAb, and analyte mAb. VRC01 is an isotype control mAb that does not bind GP. mAb114 binds the GP trimer from the top (if GP is considered oriented so that the viral membrane is at the bottom). 13C6 binds to the medial portion of the glycan cap and mAb166 binds to the lateral portion of the glycan cap. KZ52 and S1-4 A09 bind at the base of the GP trimer; KZ52 binds to one protomer of the GP trimer and S1-4 A09 makes contacts with two adjacent GP protomers simultaneously. (Top, EboV.YD.01-EboV.YD.04; Bottom, EboV.YD.05-EboV.YD.08)

FIG. 12: Kinetics of EboV.YD.01 binding to EBOV GPΔMuc. Binding to EBOV GPΔMuc was measured by biolayer interferometry. kₒₙ, k_{off}, and K_{D} values were calculated based on a global, nonlinear, least squares, 1:1 binding model curve fit with the assumption of fully reversible binding.

FIG. 13: Neutralization of pseudotyped EBOV GP particles by EboV.YD.01-04. Lentivirus particles bearing EBOV GP were incubated with antibody for 1 hour at the indicated concentrations prior to addition to 293T cells. Percent infection was determined 72 hours later by measurement of luciferase reporter gene expression. Data is presented normalized to that of negative control antibody (VRC01).

FIGS. 14A-14B: Binding of EboV.YD.02 to GPΔMuc. (FIG. 14A) Yeast from probe positive sort gates was diluted and a single colony was purified by limiting dilutions. The colony was grown in culture and binding to GPΔMuc-APC probe was confirmed using flow cytometry. (FIG. 14B) The sequence of the expressed antibody in the yeast from FIG. 14A was obtained, cloned into full IgG expression vectors, and the antibody expressed and purified. Biolayer interferometry was used to assess binding of the purified IgG to GPΔMuc. GPΔMuc was coupled to amine-reactive 2nd generation biosensors and binding of the indicated monoclonal antibodies was determined. Shown are representative binding curves from 13 replicates.

FIG. 15: Kinetics of EboV.YD.02 binding to EBOV GPΔMuc. Binding to EBOV GPΔMuc was measured by biolayer interferometry. kₒₙ, k_{off}, and K_{D} values were calculated based on a global, nonlinear, least squares, 1:1 binding model curve fit with the assumption of fully reversible binding.

FIGS. 16A-16B: Binding of EboV.YD.03 to GPΔMuc. (FIG. 16A) Yeast from probe positive sort gates was diluted and a single colony was purified by limiting dilutions. The colony was grown in culture and binding to GPΔMuc-APC probe was confirmed using flow cytometry. (FIG. 16B) The sequence of the expressed antibody in the yeast from FIG. 16A was obtained, cloned into full IgG expression vectors, and the antibody was expressed and purified. Biolayer interferometry was used to assess binding of the purified IgG to GPΔMuc. GPΔMuc was coupled to amine-reactive 2nd generation biosensors and binding of the indicated monoclonal antibodies was determined. Shown are representative binding curves from 13 replicates.

FIG. 17: Kinetics of EboV.YD.03 binding to EBOV GPΔMuc. Binding to EBOV GPΔMuc was measured by biolayer interferometry. kₒₙ, k_{off}, and K_{D} values were calculated based on a global, nonlinear, least squares, 1:1 binding model curve fit with the assumption of fully reversible binding.

FIGS. 18A-18B: Binding of EboV.YD.04 to GPΔMuc. (FIG. 18A) Yeast from probe positive sort gates was diluted and a single colony was purified by limiting dilutions. The colony was grown in culture and binding to GPΔMuc-APC probe was confirmed using flow cytometry. (FIG. 18B) The sequence of the expressed antibody in the yeast from FIG. 18A was obtained, cloned into full IgG expression vectors, and the antibody was expressed and purified. Biolayer interferometry was used to assess binding of the purified IgG to GPΔMuc. GPΔMuc was coupled to amine-reactive 2nd generation biosensors and binding of the indicated monoclonal antibodies was determined. Shown are representative binding curves from 13 replicates.

FIG. 19: Kinetics of EboV.YD.04 binding to EBOV GPΔMuc. Binding to EBOV GPΔMuc was measured by biolayer interferometry. kₒₙ, k_{off}, and K_{D} values were calculated based on a global, nonlinear, least squares, 1:1 binding model curve fit with the assumption of fully reversible binding.

FIGS. 20A-20B: Binding of EboV.YD.05 to GPΔMuc. (FIG. 20A) Yeast from probe positive sort gates was diluted and a single colony was purified by limiting dilutions. The colony was grown in culture and binding to GPΔMuc-APC probe was confirmed using flow cytometry. (FIG. 20B) The sequence of the expressed antibody in the yeast from FIG. 20A was obtained, cloned into full IgG expression vectors, and the antibody expressed and purified. Biolayer interferometry was used to assess binding of the purified IgG to GPΔMuc. GPΔMuc was coupled to amine-reactive 2nd generation biosensors and binding of the indicated monoclonal antibodies was determined. Shown are representative binding curves from 13 replicates.

FIG. 21: Kinetics of EboV.YD.05 binding to EBOV GPΔMuc. Binding to EBOV GPΔMuc was measured by biolayer interferometry. kₒₙ, k_{off}, and K_{D} values were calculated based on a global, nonlinear, least squares, 1:1 binding model curve fit with the assumption of fully reversible binding.

FIGS. 22A-22B: Binding of EboV.YD.06 to GPΔMuc. (FIG. 22A) Yeast from probe positive sort gates was diluted and a single colony was purified by limiting dilutions. The colony was grown in culture and binding to GPΔMuc-APC probe was confirmed using flow cytometry. (FIG. 22B) The sequence of the expressed antibody in the yeast from FIG. 22A was obtained, cloned into full IgG expression vectors, and the antibody was expressed and purified. Biolayer interferometry was used to assess binding of the purified IgG to GPΔMuc. GPΔMuc was coupled to amine-reactive 2nd generation biosensors and binding of the indicated monoclonal antibodies was determined. Shown are representative binding curves from 13 replicates.

FIG. 23: Kinetics of EboV.YD.06 binding to EBOV GPΔMuc. Binding to EBOV GPΔMuc was measured by biolayer interferometry. kₒₙ, k_{off}, and K_{D} values were calculated based on a global, nonlinear, least squares, 1:1 binding model curve fit with the assumption of fully reversible binding.

FIGS. 24A-24B: Binding of EboV.YD.07 to GPΔMuc. (FIG. 24A) Yeast from probe positive sort gates was diluted and a single colony was purified by limiting dilutions. The colony was grown in culture and binding to GPΔMuc-APC probe was confirmed using flow cytometry. (FIG. 24B) The sequence of the expressed antibody in the yeast from FIG. 24A was obtained, cloned into full IgG expression vectors, and the antibody was expressed and purified. Biolayer interferometry was used to assess binding of the purified IgG to GPΔMuc. GPΔMuc was coupled to amine-reactive 2nd generation biosensors and binding of the indicated monoclonal antibodies was determined. Shown are representative binding curves from 13 replicates.

FIG. 25 Kinetics of EboV.YD.07 binding to EBOV GPΔMuc. Binding to EBOV GPΔMuc was measured by biolayer interferometry. kₒₙ, k_{off}, and K_{D} values were calculated based on a global, nonlinear, least squares, 1:1 binding model curve fit with the assumption of fully reversible binding.

FIGS. 26A-26B: Binding of EboV.YD.08 to GPΔMuc. (FIG. 26A) Yeast from probe positive sort gates was diluted and a single colony was purified by limiting dilutions. The colony was grown in culture and binding to GPΔMuc-APC probe was confirmed using flow cytometry. (FIG. 26B) The sequence of the expressed antibody in the yeast from FIG. 26A was obtained, cloned into full IgG expression vectors, and the antibody was expressed and purified. Biolayer interferometry was used to assess binding of the purified IgG to GPΔMuc. GPΔMuc was coupled to amine-reactive 2nd generation biosensors and binding of the indicated monoclonal antibodies was determined. Shown are representative binding curves from 13 replicates.

FIG. 27: Kinetics of EboV.YD.08 binding to EBOV GPΔMuc. Binding to EBOV GPΔMuc was measured by biolayer interferometry. kₒₙ, k_{off}, and K_{D} values were calculated based on a global, nonlinear, least squares, 1:1 binding model curve fit with the assumption of fully reversible binding.

### SEQUENCE LISTING

The nucleic and amino acid sequences listed in the accompanying sequence listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. The Sequence Listing is submitted as an ASCII text file, created on Dec. 3, 2019, 31.3 KB, which is incorporated by reference herein. In the accompanying sequence listing:

SEQ ID NO: 1 is the nucleotide sequence of the EboV.YD.01 VH domain.

SEQ ID NO: 2 is the amino acid sequence of the EboV.YD.01 VH domain.

SEQ ID NO: 3 is the nucleotide sequence of the EboV.YD.01 VL domain.

SEQ ID NO: 4 is the amino acid sequence of the EboV.YD.01 VL domain.

SEQ ID NO: 5 is the nucleotide sequence of the EboV.YD.02 VH domain.

SEQ ID NO: 6 is the amino acid sequence of the EboV.YD.02 VH domain.

SEQ ID NO: 7 is the nucleotide sequence of the EboV.YD.02 VL domain.

SEQ ID NO: 8 is the amino acid sequence of the EboV.YD.02 VL domain.

SEQ ID NO: 9 is the nucleotide sequence of the EboV.YD.03 VH domain.

SEQ ID NO: 10 is the amino acid sequence of the EboV.YD.03 VH domain.

SEQ ID NO: 11 is the nucleotide sequence of the EboV.YD.03 VL domain.

SEQ ID NO: 12 is the amino acid sequence of the EboV.YD.03 VL domain.

SEQ ID NO: 13 is the nucleotide sequence of the EboV.YD.04 VH domain.

SEQ ID NO: 14 is the amino acid sequence of the EboV.YD.04 VH domain.

SEQ ID NO: 15 is the nucleotide sequence of the EboV.YD.04 VL domain.

SEQ ID NO: 16 is the amino acid sequence of the EboV.YD.04 VL domain.

SEQ ID NO: 17 is the nucleotide sequence of the EboV.YD.05 VH domain.

SEQ ID NO: 18 is the amino acid sequence of the EboV.YD.05 VH domain.

SEQ ID NO: 19 is the nucleotide sequence of the EboV.YD.05 VL domain.

SEQ ID NO: 20 is the amino acid sequence of the EboV.YD.05 VL domain.

SEQ ID NO: 21 is the nucleotide sequence of the EboV.YD.06 VH domain.

SEQ ID NO: 22 is the amino acid sequence of the EboV.YD.06 VH domain.

SEQ ID NO: 23 is the nucleotide sequence of the EboV.YD.06 VL domain.

SEQ ID NO: 24 is the amino acid sequence of the EboV.YD.06 VL domain.

SEQ ID NO: 25 is the nucleotide sequence of the EboV.YD.07 VH domain.

SEQ ID NO: 26 is the amino acid sequence of the EboV.YD.07 VH domain.

SEQ ID NO: 27 is the nucleotide sequence of the EboV.YD.07 VL domain.

SEQ ID NO: 28 is the amino acid sequence of the EboV.YD.07 VL domain.

SEQ ID NO: 29 is the nucleotide sequence of the EboV.YD.08 VH domain.

SEQ ID NO: 30 is the amino acid sequence of the EboV.YD.08 VH domain.

SEQ ID NO: 31 is the nucleotide sequence of the EboV.YD.08 VL domain.

SEQ ID NO: 32 is the amino acid sequence of the EboV.YD.08 VL domain.

SEQ ID NO: 33 is the nucleotide sequence of a linker.

SEQ ID NOs: 34-46 are nucleotide sequences of yeast display cloning primers.

SEQ ID NOs: 47-49 are nucleotide sequences of yeast display transformation primers.

SEQ ID NOs: 50 and 51 are nucleotide sequences of PCR primers.

### DETAILED DESCRIPTION

The present disclosure provides eight monoclonal antibodies that bind with high affinity to EBOV glycoprotein. Four of the eight antibodies were tested for their capacity to neutralize EBOV infection and all four (EboV.YD.01-EboV.YD.04) demonstrated high potency with an IC50 of 7 μg or less; three antibodies (EboV.YD.02, EboV.YD.03 and EboV.YD.04) exhibited an IC50 of less than 2 μg.

### I. Summary of Terms

Unless otherwise noted, technical terms are used according to conventional usage. Definitions of common terms in molecular biology may be found in Benjamin Lewin, Genes X, published by Jones & Bartlett Publishers, 2009; and Meyers et al. (eds.), The Encyclopedia of Cell Biology and Molecular Medicine, published by Wiley-VCH in 16 volumes, 2008; and other similar references.

As used herein, the singular forms “a,” “an,” and “the,” refer to both the singular as well as plural, unless the context clearly indicates otherwise. For example, the term “an antigen” includes single or plural antigens and can be considered equivalent to the phrase “at least one antigen.” As used herein, the term “comprises” means “includes.” It is further to be understood that any and all base sizes or amino acid sizes, and all molecular weight or molecular mass values, given for nucleic acids or polypeptides are approximate, and are provided for descriptive purposes, unless otherwise indicated. Although many methods and materials similar or equivalent to those described herein can be used, particular suitable methods and materials are described herein. In case of conflict, the present specification, including explanations of terms, will control. In addition, the materials, methods, and examples are illustrative only and not intended to be limiting. To facilitate review of the various embodiments, the following explanations of terms are provided:

Administration: The introduction of a composition into a subject by a chosen route. Administration can be local or systemic. For example, if the chosen route is intravenous, the composition is administered by introducing the composition into a vein of the subject. Exemplary routes of administration include, but are not limited to, oral, injection (such as subcutaneous, intramuscular, intradermal, intraperitoneal, and intravenous), sublingual, rectal, transdermal (for example, topical), intranasal, vaginal, and inhalation routes.

Agent: Any substance or any combination of substances that is useful for achieving an end or result; for example, a substance or combination of substances useful for inhibiting EBOV infection in a subject. Agents include proteins, antibodies, nucleic acid molecules, compounds, small molecules, organic compounds, inorganic compounds, or other molecules of interest. An agent can include a therapeutic agent, a diagnostic agent or a pharmaceutical agent. In some embodiments, the agent is a polypeptide agent (such as an EBOV-neutralizing antibody), or an anti-viral agent. Some agents may be useful to achieve more than one result.

Amino acid substitution: The replacement of one amino acid in a peptide with a different amino acid.

Antibody: An immunoglobulin, antigen-binding fragment, or derivative thereof, that specifically binds and recognizes an analyte (antigen) such as EBOV GP. The term “antibody” is used herein in the broadest sense and encompasses various antibody structures, including but not limited to monoclonal antibodies, polyclonal antibodies, multispecific antibodies (for example, bispecific antibodies), and antibody fragments, so long as they exhibit the desired antigen-binding activity.

Non-limiting examples of antibodies include, for example, intact immunoglobulins and variants and fragments thereof known in the art that retain binding affinity for the antigen. Examples of antibody fragments include but are not limited to Fv, Fab, Fab′, Fab′-SH, F(ab′)₂; diabodies; linear antibodies; single-chain antibody molecules (such as scFv); and multispecific antibodies formed from antibody fragments. Antibody fragments include antigen binding fragments either produced by the modification of whole antibodies or those synthesized de novo using recombinant DNA methodologies (see, for example, Kontermann and Dubel (Ed), Antibody Engineering, Vols. 1-2, 2^{nd} Ed., Springer Press, 2010).

A single-chain antibody (scFv) is a genetically engineered molecule containing the V_{H} and V_{L} domains of one or more antibody(ies) linked by a suitable polypeptide linker as a genetically fused single chain molecule (see, for example, Bird et al., Science, 242:423-426, 1988; Huston et al., Proc. Natl. Acad. Sci., 85:5879-5883, 1988; Ahmad et al., Clin. Dev. Immunol., 2012, doi:10.1155/2012/980250; Marbry, IDrugs, 13:543-549, 2010). The intramolecular orientation of the V_{H} domain and the V_{L} domain in a scFv is typically not decisive for scFvs. Thus, scFvs with both possible arrangements (V_{H} domain-linker domain-V_{L}-domain; V_{L}-domain-linker domain-V_{H}-domain) may be used.

In a dsFv the V_{H} and V_{L} have been mutated to introduce a disulfide bond to stabilize the association of the chains. Diabodies also are included, which are bivalent, bispecific antibodies in which V_{H} and V_{L} domains are expressed on a single polypeptide chain, but using a linker that is too short to allow for pairing between the two domains on the same chain, thereby forcing the domains to pair with complementary domains of another chain and creating two antigen binding sites (see, for example, Holliger et al., Proc. Natl. Acad. Sci., 90:6444-6448, 1993; Poljak et al., Structure, 2:1121-1123, 1994).

Antibodies also include genetically engineered forms such as chimeric antibodies (such as humanized murine antibodies) and heteroconjugate antibodies (such as bispecific antibodies). See also, Pierce Catalog and Handbook, 1994-1995 (Pierce Chemical Co., Rockford, Ill.); Kuby, J., Immunology, 3^{rd} Ed., W.H. Freeman & Co., New York, 1997.

An “antibody that binds to the same epitope” as a reference antibody refers to an antibody that blocks binding of the reference antibody to its antigen in a competition assay by 50% or more, and conversely, the reference antibody blocks binding of the antibody to its antigen in a competition assay by 50% or more. Antibody competition assays are well-known in the art.

An antibody may have one or more binding sites. If there is more than one binding site, the binding sites may be identical to one another or may be different. For instance, a naturally-occurring immunoglobulin has two identical binding sites, a single-chain antibody or Fab fragment has one binding site, while a bispecific or bifunctional antibody has two different binding sites.

Typically, a naturally occurring immunoglobulin has heavy (H) chains and light (L) chains interconnected by disulfide bonds. Immunoglobulin genes include the kappa, lambda, alpha, gamma, delta, epsilon and mu constant region genes, as well as the myriad immunoglobulin variable domain genes. There are two types of light chain, lambda (λ) and kappa (κ). There are five main heavy chain classes (or isotypes) which determine the functional activity of an antibody molecule: IgM, IgD, IgG, IgA and IgE.

Each heavy and light chain contains a constant region (or constant domain) and a variable region (or variable domain; see, e.g., Kindt et al. Kuby Immunology, 6^{th} ed., W.H. Freeman and Co., page 91 (2007).) In several embodiments, the V_{H} and V_{L} combine to specifically bind the antigen. In additional embodiments, only the V_{H} is required. For example, naturally occurring camelid antibodies consisting of a heavy chain only are functional and stable in the absence of light chain (see, e.g., Hamers-Casterman et al., Nature, 363:446-448, 1993; Sheriff et al., Nat. Struct. Biol., 3:733-736, 1996). Any of the disclosed antibodies can include a heterologous constant domain. For example the antibody can include constant domain that is different from a native constant domain, such as a constant domain including one or more modifications to increase half-life.

References to “V_{H}” or “VH” refer to the variable region of an antibody heavy chain, including that of an antigen binding fragment, such as Fv, scFv, dsFv or Fab. References to “V_{L}” or “VL” refer to the variable domain of an antibody light chain, including that of an Fv, scFv, dsFv or Fab.

The V_{H} and V_{L} contain a “framework” region interrupted by three hypervariable regions, also called “complementarity-determining regions” or “CDRs” (see, for example, Kabat et al., Sequences of Proteins of Immunological Interest, U.S. Department of Health and Human Services, 1991). The sequences of the framework regions of different light or heavy chains are relatively conserved within a species. The framework region of an antibody, that is the combined framework regions of the constituent light and heavy chains, serves to position and align the CDRs in three-dimensional space.

The CDRs are primarily responsible for binding to an epitope of an antigen. The CDRs of each chain are typically referred to as CDR1, CDR2, and CDR3 (from the N-terminus to C-terminus), and are also typically identified by the chain in which the particular CDR is located. Thus, a V_{H} CDR3 is the CDR3 from the V_{H} of the antibody in which it is found, whereas a V_{L} CDR1 is the CDR1 from the V_{L} of the antibody in which it is found. Light chain CDRs are sometimes referred to as LCDR1, LCDR2, and LCDR3. Heavy chain CDRs are sometimes referred to as HCDR1, HCDR2, and HCDR3. The amino acid sequence boundaries of a given CDR can be readily determined using any of a number of well-known schemes, including those described by Kabat et al. (“Sequences of Proteins of Immunological Interest,” 5th Ed. Public Health Service, National Institutes of Health, Bethesda, Md., 1991; “Kabat” numbering scheme), Al-Lazikani et al., (JMB 273,927-948, 1997; “Chothia” numbering scheme), Lefranc et al. (“IMGT unique numbering for immunoglobulin and T cell receptor variable domains and Ig superfamily V-like domains,” Dev. Comp. Immunol., 27:55-77, 2003; “IMGT” numbering scheme), and Kunik et al. (see Kunik et al., PLoS Comput Biol 8:e1002388, 2012; and Kunik et al., Nucleic Acids Res 40(Web Server issue):W521-524, 2012; “Paratome CDRs”). The Kabat, Paratome and IMGT databases are maintained online.

A “monoclonal antibody” is an antibody obtained from a population of substantially homogeneous antibodies, that is, the individual antibodies comprising the population are identical and/or bind the same epitope, except for possible variant antibodies, for example, containing naturally occurring mutations or mutations arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies may be made by a variety of techniques, including but not limited to the hybridoma method, recombinant DNA methods, phage-display methods, yeast display methods, and methods utilizing transgenic animals containing all or part of the human immunoglobulin loci, such methods and other exemplary methods for making monoclonal antibodies being described herein. In some examples monoclonal antibodies are isolated from a subject. Monoclonal antibodies can have conservative amino acid substitutions which have substantially no effect on antigen binding or other immunoglobulin functions. (See, for example, Harlow & Lane, Antibodies, A Laboratory Manual, 2^{nd} ed. Cold Spring Harbor Publications, New York (2013).)

A “humanized” antibody or antigen binding fragment includes a human framework region and one or more CDRs from a non-human (such as a mouse, rat, or synthetic) antibody or antigen binding fragment. The non-human antibody or antigen binding fragment providing the CDRs is termed a “donor,” and the human antibody or antigen binding fragment providing the framework is termed an “acceptor.” In one embodiment, all the CDRs are from the donor immunoglobulin in a humanized immunoglobulin. Constant regions need not be present, but if they are, they can be substantially identical to human immunoglobulin constant regions, such as at least about 85-90%, such as about 95% or more identical. Hence, all parts of a humanized antibody or antigen binding fragment, except possibly the CDRs, are substantially identical to corresponding parts of natural human antibody sequences.

A “chimeric antibody” is an antibody which includes sequences derived from two different antibodies, which typically are of different species. In some examples, a chimeric antibody includes one or more CDRs and/or framework regions from one human antibody and CDRs and/or framework regions from another human antibody.

A “fully human antibody” or “human antibody” is an antibody which includes sequences from (or derived from) the human genome, and does not include sequence from another species. In some embodiments, a human antibody includes CDRs, framework regions, and (if present) an Fc region from (or derived from) the human genome. Human antibodies can be identified and isolated using technologies for creating antibodies based on sequences derived from the human genome, for example by phage display or using transgenic animals (see, for example, Barbas et al., Phage display: A Laboratory Manuel. 1^{st} Ed. New York: Cold Spring Harbor Laboratory Press, 2004. Print.; Lonberg, Nat. Biotech., 23: 1117-1125, 2005; Lonenberg, Curr. Opin. Immunol., 20:450-459, 2008)

Antibody or antigen binding fragment that neutralizes EBOV: An antibody or antigen binding fragment that specifically binds to EBOV GP in such a way as to inhibit a biological function associated with EBOV GP (such as binding to its target receptor). In several embodiments, an antibody or antigen binding fragment that neutralizes EBOV reduces the infectious titer of EBOV. In some embodiments, an antibody or antigen binding fragment that specifically binds to EBOV GP can neutralize two or more (such as 3, 4, 5, 6, 7, 8, 9, 10, or more) strains of EBOV.

Biological sample: A sample obtained from a subject. Biological samples include all clinical samples useful for detection of disease or infection (for example, EVD or EBOV infection) in subjects, including, but not limited to, cells, tissues, and bodily fluids, such as blood, derivatives and fractions of blood (such as serum), cerebrospinal fluid; as well as biopsied or surgically removed tissue, for example tissues that are unfixed, frozen, or fixed in formalin or paraffin. In a particular example, a biological sample is obtained from a subject having or suspected of having an Ebola infection.

Bispecific antibody: A recombinant molecule composed of two different antigen binding domains that consequently binds to two different antigenic epitopes. Bispecific antibodies include chemically or genetically linked molecules of two antigen-binding domains. The antigen binding domains can be linked using a linker. The antigen binding domains can be monoclonal antibodies, antigen-binding fragments (for example, Fab, scFv), or combinations thereof. A bispecific antibody can include one or more constant domains, but does not necessarily include a constant domain. Similarly, a multi-specific antibody is a recombinant protein that includes antigen-binding fragments of at least two different monoclonal antibodies, such as two, three or four different monoclonal antibodies.

Conditions sufficient to form an immune complex: Conditions which allow an antibody or antigen binding fragment to bind to its cognate epitope to a detectably greater degree than, and/or to the substantial exclusion of, binding to substantially all other epitopes. Conditions sufficient to form an immune complex are dependent upon the format of the binding reaction and typically are those utilized in immunoassay protocols or those conditions encountered in vivo. See Harlow & Lane, Antibodies, A Laboratory Manual, 2^{nd} ed. Cold Spring Harbor Publications, New York (2013) for a description of immunoassay formats and conditions. The conditions employed in the methods are “physiological conditions” which include reference to conditions (such as temperature, osmolarity, pH) that are typical inside a living mammal or a mammalian cell. While it is recognized that some organs are subject to extreme conditions, the intra-organismal and intracellular environment normally lies around pH 7 (for example, from pH 6.0 to pH 8.0, more typically pH 6.5 to 7.5), contains water as the predominant solvent, and exists at a temperature above 0° C. and below 50° C. Osmolarity is within the range that is supportive of cell viability and proliferation.

The formation of an immune complex can be detected through conventional methods, for instance immunohistochemistry, immunoprecipitation, flow cytometry, immunofluorescence microscopy, ELISA, immunoblotting (for example, Western blot), magnetic resonance imaging, CT scans, X-ray and affinity chromatography. Immunological binding properties of selected antibodies may be quantified using methods well known in the art.

Conjugate: A complex of two molecules linked together, for example, linked together by a covalent bond. In one embodiment, an antibody is linked to an effector molecule; for example, an antibody that specifically binds to EBOV GP covalently linked to an effector molecule. The linkage can be by chemical or recombinant means. In one embodiment, the linkage is chemical, wherein a reaction between the antibody moiety and the effector molecule has produced a covalent bond formed between the two molecules to form one molecule. A peptide linker (short peptide sequence) can optionally be included between the antibody and the effector molecule. Because conjugates can be prepared from two molecules with separate functionalities, such as an antibody and an effector molecule, they are also sometimes referred to as “chimeric molecules.”

Conservative variants: “Conservative” amino acid substitutions are those substitutions that do not substantially affect or decrease a function of a protein, such as the ability of the protein to interact with a target protein. For example, an EBOV-specific antibody can include up to 1, 2, 3, 4, 5, 6, 7, 8, 9, or up to 10 conservative substitutions compared to a reference antibody sequence and retain specific binding activity for EBOV antigen, and/or EBOV neutralization activity. The term conservative variation also includes the use of a substituted amino acid in place of an unsubstituted parent amino acid.

Furthermore, individual substitutions, deletions or additions which alter, add or delete a single amino acid or a small percentage of amino acids (for instance less than 5%, in some embodiments less than 1%) in an encoded sequence are conservative variations where the alterations result in the substitution of an amino acid with a chemically similar amino acid.

Conservative amino acid substitution tables providing functionally similar amino acids are known. The following six groups are examples of amino acids that are considered to be conservative substitutions for one another:
1) Alanine (A), Serine (S), Threonine (T);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (W).

Non-conservative substitutions are those that reduce an activity or function of the EBOV-specific antibody, such as the ability to specifically bind to EBOV GP. For instance, if an amino acid residue is essential for a function of the protein, even an otherwise conservative substitution may disrupt that activity. Thus, a conservative substitution does not alter the basic function of a protein of interest.

Contacting: Placement in direct physical association; includes both in solid and liquid form, which can take place either in vivo or in vitro. Contacting includes contact between one molecule and another molecule, for example the amino acid on the surface of one polypeptide, such as an antigen, that contacts another polypeptide, such as an antibody. Contacting can also include contacting a cell for example by placing an antibody in direct physical association with a cell.

Control: A reference standard. In some embodiments, the control is a negative control sample obtained from a healthy patient. In other embodiments, the control is a positive control sample obtained from a patient diagnosed with EBOV infection. In still other embodiments, the control is a historical control or standard reference value or range of values (such as a previously tested control sample, such as a group of EBOV patients with known prognosis or outcome, or group of samples that represent baseline or normal values).

A difference between a test sample and a control can be an increase or conversely a decrease. The difference can be a qualitative difference or a quantitative difference, for example a statistically significant difference. In some examples, a difference is an increase or decrease, relative to a control, of at least about 5%, such as at least about 10%, at least about 20%, at least about 30%, at least about 40%, at least about 50%, at least about 60%, at least about 70%, at least about 80%, at least about 90%, at least about 100%, at least about 150%, at least about 200%, at least about 250%, at least about 300%, at least about 350%, at least about 400%, at least about 500%, or greater than 500%.

Degenerate variant: In the context of the present disclosure, a “degenerate variant” refers to a polynucleotide encoding a protein (for example, an antibody that specifically binds EBOV GP) that includes a sequence that is degenerate as a result of the genetic code. There are twenty natural amino acids, most of which are specified by more than one codon. Therefore, all degenerate nucleotide sequences are included as long as the amino acid sequence of the antibody that binds EBOV GP encoded by the nucleotide sequence is unchanged.

Detectable label: A detectable molecule (also known as a detectable marker) that is conjugated directly or indirectly to a second molecule, such as an antibody, to facilitate detection of the second molecule. For example, the detectable label can be capable of detection by ELISA, spectrophotometry, flow cytometry, microscopy or diagnostic imaging techniques (such as CT scans, MRIs, ultrasound, fiberoptic examination, and laparoscopic examination). Specific, non-limiting examples of detectable labels include fluorophores, chemiluminescent agents, enzymatic linkages, radioactive isotopes, nucleic acids (such as DNA barcodes), and heavy metals or compounds (for example super paramagnetic iron oxide nanocrystals for detection by MRI). In one example, a “labeled antibody” refers to incorporation of another molecule in the antibody. For example, the label is a detectable label, such as the incorporation of a radiolabeled amino acid or attachment to a polypeptide of biotinyl moieties that can be detected by marked avidin (for example, streptavidin containing a fluorescent marker or enzymatic activity that can be detected by optical or colorimetric methods). Various methods of labeling polypeptides and glycoproteins are known in the art and may be used. Examples of labels for polypeptides include, but are not limited to, the following: radioisotopes or radionuclides (such as ³⁵S or ¹³¹I) fluorescent labels (such as fluorescein isothiocyanate (FITC), rhodamine, lanthanide phosphors), enzymatic labels (such as horseradish peroxidase, beta-galactosidase, luciferase, alkaline phosphatase), chemiluminescent markers, biotinyl groups, predetermined polypeptide epitopes recognized by a secondary reporter (such as a leucine zipper pair sequences, binding sites for secondary antibodies, metal binding domains, epitope tags), or magnetic agents, such as gadolinium chelates. In some embodiments, labels are attached by spacer arms of various lengths to reduce potential steric hindrance. Methods for using detectable labels and guidance in the choice of detectable labels appropriate for various purposes are discussed for example in Sambrook et al. (Molecular Cloning: A Laboratory Manual, 4^{th} ed, Cold Spring Harbor, N.Y., 2012) and Ausubel et al. (In Current Protocols in Molecular Biology, John Wiley & Sons, New York, through supplement 104, 2013).

Ebola Virus (EBOV): An enveloped, non-segmented, negative-sense, single-stranded RNA virus that causes Ebola virus disease (EVD), formerly known as Ebola hemorrhagic fever (EHF), in humans. EBOV spreads through human-to-human transmission, with infection resulting from direct contact with blood, secretions, organs or other bodily fluids of infected people, and indirect contact with environments contaminated by such fluids (see, for example, Baize et al., N Engl J Med., 371, 1418-1425, 2014).

The symptoms of EBOV infection and disease are well-known. Briefly, in humans, EBOV has an initial incubation period of 2 to 21 days (7 days on average, depending on the strain) followed by a rapid onset of non-specific symptoms such as fever, extreme fatigue, gastrointestinal complaints, abdominal pain, anorexia, headache, myalgia and/or arthralgia. These initial symptoms last for about 2 to 7 days after which more severe symptoms related to hemorrhagic fever occur, including hemorrhagic rash, epistaxis, mucosal bleeding, hematuria, hemoptysis, hematemesis, melena, conjunctival hemorrhage, tachypnea, confusion, somnolence, and hearing loss. In general, the symptoms last for about 7 to 14 days after which recovery may occur. Death can occur 6 to 16 days after the onset of symptoms (Geisbert and Jahrling, Nat Med., 10, S110-21. 2004; Hensley et al., Curr Mol Med, 5, 761-72, 2005). People are infectious as long as their blood and secretions contain the virus; the virus was isolated from semen 61 days after onset of illness in a man who was infected in a laboratory (Baize et al., N Engl J Med., 371, 1418-1425, 2014).

IgM antibodies to the virus appear 2 to 9 days after infection whereas IgG antibodies appear approximately 17 to 25 days after infection, which coincides with the recovery phase. In survivors of EVD, both humoral and cellular immunity are detected, however, their relative contribution to protection is unknown (Sullivan et al., J Virol, 77:9733-7, 2003).

Five distinct EBOV species are known, including Bundibugyo (BDBV), Reston (RESTV), Sudan (SUDV), Taï Forest (TAFV), and Zaire (ZEBOV) (Kuhn, J. H., et al., Arch Virol, 2013. 158(1): p. 301-11). BDBV, ZEBOV, and SUDV have been associated with large outbreaks of EVD in Africa and reported case fatality rates of up to 90%.

The EBOV genome includes about 19K nucleotides, which encode seven structural proteins including NP (a nucleoprotein), VP35 (a polymerase cofactor), VP30 (a transcription activator), VP24, L (a RNA polymerase), and GP (a glycoprotein).

EBOV glycoprotein (GP): The virion-associated transmembrane glycoprotein of EBOV is initially synthesized as a precursor protein of about 675 amino acids in size, designated GP₀. Individual GP₀ polypeptides form a homotrimer and undergo glycosylation within the Golgi apparatus as well as processing to remove the signal peptide, and cleavage by a cellular protease between approximately positions 500/501 to generate separate GP₁ and GP₂ polypeptide chains, which remain associated as GP₁/GP₂ protomers within the homotrimer. The extracellular GP₁ polypeptide (approximately 140 kDa) is derived from the amino-terminal portion of the GP₀ precursor, and the GP₂ polypeptide (approximately 26 kDa), which includes extracellular, transmembrane, and cytosolic domains, is derived from the carboxyl-terminal portion of the GP₀ precursor. GP₁ is responsible for attachment to new host cells while GP₂ mediates fusion with those cells.

A splice variant of the gene encoding EBOV GP encodes a soluble glycoprotein (sGP) that is secreted from the viral host cell (Volchkov et al., Virology, 245, 110-119, 1998). sGP and GP₁ are identical in their first 295 N-terminal amino acids, whereas the remaining 69 C-terminal amino acids of sGP and 206 amino acids of GP₁ are encoded by different reading frames. It has been suggested that secreted sGP may effectively bind antibodies that might otherwise be protective (see, e.g., Sanchez el al., Proc. Natl. Acad. Sci. U.S.A., 93, 3602-3607, 1996; and Volchkov et al., Virology, 245, 110-119, 1998).

Comparisons of the predicted amino acid sequences for the GPs of the different EBOV strains show conservation of amino acids in the amino-terminal and carboxy-terminal regions with a highly variable region in the middle of the protein (Feldmann el al., Virus Res. 24: 1-19, 1992). The GP of Ebola viruses is highly glycosylated and contains both N-linked and O-linked carbohydrates that contribute up to 50% of the molecular weight of the protein. Most of the glycosylation sites are found in the central variable region of GP.

Effector molecule: A molecule intended to have or produce a desired effect; for example, a desired effect on a cell to which the effector molecule is targeted. Effector molecules can include, for example, polypeptides, small molecules, drugs, toxins, therapeutic agents, detectable labels, nucleic acids, lipids, nanoparticles, carbohydrates or recombinant viruses. In one non-limiting example, the effector molecule is a toxin. Some effector molecules may have or produce more than one desired effect.

Epitope: An antigenic determinant. These are particular chemical groups or peptide sequences on a molecule that are antigenic (for example, sequences that elicit a specific immune response). An antibody specifically binds a particular antigenic epitope on a polypeptide. In some examples a disclosed antibody specifically binds to an epitope on EBOV GP.

Expression: Transcription or translation of a nucleic acid sequence. For example, an encoding nucleic acid sequence (such as a gene) can be expressed when its DNA is transcribed into an RNA or RNA fragment, which in some examples is processed to become mRNA. An encoding nucleic acid sequence (such as a gene) may also be expressed when its mRNA is translated into an amino acid sequence, such as a protein or a protein fragment. In a particular example, a heterologous gene is expressed when it is transcribed into an RNA. In another example, a heterologous gene is expressed when its RNA is translated into an amino acid sequence. Regulation of expression can include controls on transcription, translation, RNA transport and processing, degradation of intermediary molecules such as mRNA, or through activation, inactivation, compartmentalization or degradation of specific protein molecules after they are produced.

Expression control sequences: Nucleic acid sequences that regulate the expression of a heterologous nucleic acid sequence to which it is operatively linked Expression control sequences are operatively linked to a nucleic acid sequence when the expression control sequences control and regulate the transcription and, as appropriate, translation of the nucleic acid sequence. Thus expression control sequences can include appropriate promoters, enhancers, transcription terminators, a start codon (ATG) in front of a protein-encoding gene, splicing signal for introns, maintenance of the correct reading frame of that gene to permit proper translation of mRNA, and stop codons. The term “control sequences” is intended to include, at a minimum, components whose presence can influence expression, and can also include additional components whose presence is advantageous, for example, leader sequences and fusion partner sequences. Expression control sequences can include a promoter.

A promoter is a minimal sequence sufficient to direct transcription. Also included are those promoter elements which are sufficient to render promoter-dependent gene expression controllable for cell-type specific, tissue-specific, or inducible by external signals or agents; such elements may be located in the 5′ or 3′ regions of the gene. Both constitutive and inducible promoters are included (see for example, Bitter et al., Methods in Enzymology 153:516-544, 1987). For example, when cloning in bacterial systems, inducible promoters such as pL of bacteriophage lambda, plac, ptrp, ptac (ptrp-lac hybrid promoter) and the like may be used. In one embodiment, when cloning in mammalian cell systems, promoters derived from the genome of mammalian cells (such as metallothionein promoter) or from mammalian viruses (such as the retrovirus long terminal repeat; the adenovirus late promoter; the vaccinia virus 7.5K promoter) can be used. Promoters produced by recombinant DNA or synthetic techniques may also be used to provide for transcription of the nucleic acid sequences.

A polynucleotide can be inserted into an expression vector that contains a promoter sequence which facilitates the efficient transcription of the inserted genetic sequence of the host. The expression vector typically contains an origin of replication, a promoter, as well as specific nucleic acid sequences that allow phenotypic selection of the transformed cells.

Expression vector: A vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (for example, naked or contained in liposomes) and viruses (for example, lentiviruses, retroviruses, adenoviruses, and adeno-associated viruses) that incorporate the recombinant polynucleotide.

Fc region: The polypeptide including the constant region of an antibody excluding the first constant region immunoglobulin domain. Fc region generally refers to the last two constant region immunoglobulin domains of IgA, IgD, and IgG, and the last three constant region immunoglobulin domains of IgE and IgM. An Fc region may also include part or all of the flexible hinge N-terminal to these domains. For IgA and IgM, an Fc region may or may not include the tailpiece, and may or may not be bound by the J chain. For IgG, the Fc region includes immunoglobulin domains Cgamma2 and Cgamma3 (Cγ2 and Cγ3) and the lower part of the hinge between Cgamma1 (Cγ1) and Cγ2. Although the boundaries of the Fc region may vary, the human IgG heavy chain Fc region is usually defined to include residues C226 or P230 to its carboxyl-terminus, wherein the numbering is according to the EU index as in Kabat. For IgA, the Fc region includes immunoglobulin domains Calpha2 and Calpha3 (Cα2 and Cα3) and the lower part of the hinge between Calpha1 (Cal) and Cα2. In some embodiments herein, the disclosed antibodies comprise a heterologous Fc region or heterologous constant domain. For example, the antibody comprises a Fc region or constant domain that is different from a native Fc region or constant domain, such as a Fc region or constant domain including one or more modifications (such as the “LS” mutations) to increase half-life.

Heterologous: Originating from a different genetic source. A nucleic acid molecule that is heterologous to a cell originated from a genetic source other than the cell in which it is expressed.

IgG: A polypeptide belonging to the class or isotype of antibodies that are substantially encoded by a recognized immunoglobulin gamma gene. In humans, this class comprises IgG₁, IgG₂, IgG₃, and IgG₄. In mice, this class comprises IgG₁, IgG₂ₐ, IgG_{2b}, IgG₃.

Immune complex: The binding of antibody or antigen binding fragment (such as a scFv) to a soluble antigen forms an immune complex. The formation of an immune complex can be detected through conventional methods, for instance immunohistochemistry, immunoprecipitation, flow cytometry, immunofluorescence microscopy, ELISA, immunoblotting (for example, Western blot), magnetic resonance imaging, CT scans, X-ray and affinity chromatography. Immunological binding properties of selected antibodies may be quantified using methods well known in the art.

Isolated: A biological component (such as a nucleic acid, peptide, protein or protein complex, for example an antibody) that has been substantially separated, produced apart from, or purified away from other biological components in the cell of the organism in which the component naturally occurs, that is, other chromosomal and extra-chromosomal DNA and RNA, and proteins. Thus, isolated nucleic acids, peptides and proteins include nucleic acids and proteins purified by standard purification methods. The term also embraces nucleic acids, peptides and proteins prepared by recombinant expression in a host cell, as well as, chemically synthesized nucleic acids. An isolated nucleic acid, peptide or protein, for example an antibody, can be at least 50%, at least 60%, at least 70%, at least 80%, at least 90%, at least 95%, at least 96%, at least 97%, at least 98%, or at least 99% pure.

Linker: A bi-functional molecule that can be used to link two molecules into one contiguous molecule, for example, to link an effector molecule to an antibody. In some embodiments, the provided conjugates include a linker between the effector molecule or detectable marker and an antibody. In some cases, a linker is a peptide within an antigen binding fragment (such as an Fv fragment) which serves to indirectly bond the variable heavy chain to the variable light chain. Non-limiting examples of peptide linkers include glycine, serine, and glycine-serine linkers.

The terms “conjugating,” “joining,” “bonding,” or “linking” can refer to making two molecules into one contiguous molecule; for example, linking two polypeptides into one contiguous polypeptide, or covalently attaching an effector molecule or detectable marker radionuclide or other molecule to a polypeptide, such as an scFv. In the specific context, the terms include reference to joining a ligand, such as an antibody moiety, to an effector molecule. The linkage can be either by chemical or recombinant means. “Chemical means” refers to a reaction between the antibody moiety and the effector molecule such that there is a covalent bond formed between the two molecules to form one molecule.

Nucleic acid (molecule or sequence): A deoxyribonucleotide or ribonucleotide polymer or combination thereof including without limitation, cDNA, mRNA, genomic DNA, and synthetic (such as chemically synthesized) DNA or RNA. The nucleic acid can be double stranded (ds) or single stranded (ss). Where single stranded, the nucleic acid can be the sense strand or the antisense strand. Nucleic acids can include natural nucleotides (such as A, T/U, C, and G), and can include analogs of natural nucleotides, such as labeled nucleotides. “Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA produced by that gene produces the protein in a cell or other biological system. Both the coding strand, the nucleotide sequence of which is identical to the mRNA sequence and is usually provided in sequence listings, and non-coding strand, used as the template for transcription, of a gene or cDNA can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns. “cDNA” refers to a DNA that is complementary or identical to an mRNA, in either single stranded or double stranded form.

Operably linked: A first nucleic acid sequence is operably linked with a second nucleic acid sequence when the first nucleic acid sequence is placed in a functional relationship with the second nucleic acid sequence. For instance, a promoter, such as the CMV promoter, is operably linked to a coding sequence if the promoter affects the transcription or expression of the coding sequence. Generally, operably linked DNA sequences are contiguous and, where necessary to join two protein-coding regions, in the same reading frame.

Pharmaceutically acceptable carriers: The pharmaceutically acceptable carriers of use are conventional. Remington's Pharmaceutical Science, 22th ed., Pharmaceutical Press, London, UK (2012), describes compositions and formulations suitable for pharmaceutical delivery of the disclosed agents.

In general, the nature of the carrier will depend on the particular mode of administration being employed. For instance, parenteral formulations usually include injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, added preservatives (such as on-natural preservatives), and pH buffering agents and the like, for example sodium acetate or sorbitan monolaurate. In particular examples, the pharmaceutically acceptable carrier is sterile and suitable for parenteral administration to a subject for example, by injection. In some embodiments, the active agent and pharmaceutically acceptable carrier are provided in a unit dosage form such as a pill or in a selected quantity in a vial. Unit dosage forms can include one dosage or multiple dosages (for example, in a vial from which metered dosages of the agents can selectively be dispensed).

Polypeptide: A polymer in which the monomers are amino acid residues that are joined together through amide bonds. When the amino acids are alpha-amino acids, either the L-optical isomer or the D-optical isomer can be used, the L-isomers being preferred. The terms “polypeptide” or “protein” as used herein are intended to encompass any amino acid sequence and include modified sequences such as glycoproteins. A polypeptide includes both naturally occurring proteins, as well as those that are recombinantly or synthetically produced. A polypeptide has an amino terminal (N-terminal) end and a carboxy-terminal end. In some embodiments, the polypeptide is a disclosed antibody or a fragment thereof.

Purified: The term purified does not require absolute purity; rather, it is intended as a relative term. Thus, for example, a purified peptide preparation is one in which the peptide or protein (such as an antibody) is more enriched than the peptide or protein is in its natural environment within a cell. In one embodiment, a preparation is purified such that the protein or peptide represents at least 50% of the total peptide or protein content of the preparation, such as at least 80%, at least 90%, at least 95% or greater of the total peptide or protein content.

Recombinant: A recombinant nucleic acid is one that has a sequence that is not naturally occurring or has a sequence that is made by an artificial combination of two otherwise separated segments of sequence. This artificial combination can be accomplished by chemical synthesis or by the artificial manipulation of isolated segments of nucleic acids, for example, by genetic engineering techniques. A recombinant protein is one that has a sequence that is not naturally occurring or has a sequence that is made by an artificial combination of two otherwise separated segments of sequence. In several embodiments, a recombinant protein is encoded by a heterologous (for example, recombinant) nucleic acid that has been introduced into a host cell, such as a bacterial or eukaryotic cell. The nucleic acid can be introduced, for example, on an expression vector having signals capable of expressing the protein encoded by the introduced nucleic acid or the nucleic acid can be integrated into the host cell chromosome.

Sequence identity: The similarity between amino acid or nucleic acid sequences is expressed in terms of the similarity between the sequences, otherwise referred to as sequence identity. Sequence identity is frequently measured in terms of percentage identity (or similarity or homology); the higher the percentage, the more similar the two sequences are. Homologs or variants of a polypeptide or nucleic acid molecule will possess a relatively high degree of sequence identity when aligned using standard methods.

Methods of alignment of sequences for comparison are well known in the art. Various programs and alignment algorithms are described in: Smith and Waterman, Adv. Appl. Math. 2:482, 1981; Needleman and Wunsch, J. Mol. Biol. 48:443, 1970; Pearson and Lipman, Proc. Natl. Acad. Sci. U.S.A. 85:2444, 1988; Higgins and Sharp, Gene 73:237, 1988; Higgins and Sharp, CABIOS 5:151, 1989; Corpet et al., Nucleic Acids Research 16:10881, 1988; and Pearson and Lipman, Proc. Natl. Acad. Sci. U.S.A. 85:2444, 1988. Altschul et al., Nature Genet. 6:119, 1994, presents a detailed consideration of sequence alignment methods and homology calculations.

The NCBI Basic Local Alignment Search Tool (BLAST) (Altschul et al., J. Mol. Biol. 215:403, 1990) is available from several sources, including the National Center for Biotechnology Information (NCBI, Bethesda, Md.) and on the internet, for use in connection with the sequence analysis programs blastp, blastn, blastx, tblastn and tblastx. A description of how to determine sequence identity using this program is available on the NCBI website on the internet.

Homologs and variants of a polypeptide are typically characterized by possession of at least about 75%, for example at least about 80%, 85%, 90%, 91%, 92%, 93%, 94%, 95%, 96%, 97%, 98% or 99% sequence identity counted over the full length alignment with the amino acid sequence of interest. Proteins with even greater similarity to the reference sequences will show increasing percentage identities when assessed by this method, such as at least 80%, at least 85%, at least 90%, at least 95%, at least 98%, or at least 99% sequence identity. When less than the entire sequence is being compared for sequence identity, homologs and variants will typically possess at least 80% sequence identity over short windows of 10-20 amino acids, and may possess sequence identities of at least 85% or at least 90% or 95% depending on their similarity to the reference sequence. Methods for determining sequence identity over such short windows are available at the NCBI website on the internet. One of skill in the art will appreciate that these sequence identity ranges are provided for guidance only; it is entirely possible that strongly significant homologs could be obtained that fall outside of the ranges provided.

As used herein, reference to “at least 90% identity” refers to “at least 90%, at least 91%, at least 92%, at least 93%, at least 94%, at least 95%, at least 96%, at least 97%, at least 98%, at least 99%, or even 100% identity” to a specified reference sequence.

Specifically bind: When referring to an antibody or antigen binding fragment, refers to a binding reaction which determines the presence of a target protein, peptide, or polysaccharide in the presence of a heterogeneous population of proteins and other biologics. Thus, under designated conditions, an antibody binds preferentially to a particular target protein, peptide or polysaccharide (such as an antigen present on the surface of a pathogen, for example EBOV GP) and does not bind in a significant amount to other proteins or polysaccharides present in the sample or subject. With reference to an antibody-antigen complex, specific binding of the antigen and antibody has a K_{d} of less than about 10⁻⁷ Molar, such as less than about 10⁻⁸ Molar, 10⁻⁹, or even less than about 10⁻¹⁰ Molar.

K_{d} refers to the dissociation constant for a given interaction, such as a polypeptide ligand interaction or an antibody antigen interaction. For example, for the bimolecular interaction of an antibody or antigen binding fragment (such as EboV.YD.01 or an antigen binding fragment thereof) and an antigen (such as EBOV GP) it is the concentration of the individual components of the bimolecular interaction divided by the concentration of the complex.

The antibodies disclosed herein specifically bind to a defined target (or multiple targets, in the case of a bispecific antibody). Thus, an antibody that specifically binds to an epitope on EBOV GP is an antibody that binds substantially to EBOV GP, including cells or tissue expressing EBOV GP, substrate to which the EBOV GP is attached, or EBOV GP in a biological specimen. It is, of course, recognized that a certain degree of non-specific interaction may occur between an antibody or conjugate including an antibody (such as an antibody that specifically binds EBOV GP or conjugate including such antibody) and a non-target (such as a cell that does not express EBOV GP). Typically, specific binding results in a much stronger association between the antibody and protein or cells bearing the antigen than between the antibody and protein or cells lacking the antigen. Specific binding typically results in greater than 2-fold, such as greater than 5-fold, greater than 10-fold, or greater than 100-fold increase in amount of bound antibody (per unit time) to a protein including the epitope or cell or tissue expressing the target epitope as compared to a protein or cell or tissue lacking this epitope. Specific binding to a protein under such conditions requires an antibody that is selected for its specificity for a particular protein. A variety of immunoassay formats are appropriate for selecting antibodies or other ligands specifically immunoreactive with a particular protein. For example, solid-phase ELISA immunoassays are routinely used to select monoclonal antibodies specifically immunoreactive with a protein. See Harlow & Lane, Antibodies, A Laboratory Manual, 2^{nd} ed., Cold Spring Harbor Publications, New York (2013), for a description of immunoassay formats and conditions that can be used to determine specific immunoreactivity.

Subject: Living multi-cellular vertebrate organisms, a category that includes human and non-human mammals. In an example, a subject is a human. In an additional example, a subject is selected that is in need of inhibiting of an EBOV infection. For example, the subject is either uninfected and at risk of EBOV infection or is infected in need of treatment.

Synthetic: Produced by artificial means in a laboratory, for example a synthetic nucleic acid or protein (for example, an antibody) can be chemically synthesized in a laboratory.

Therapeutically effective amount: The amount of agent, such as a disclosed EBOV GP specific antibody or antigen binding fragment that is sufficient to prevent, treat (including prophylaxis), reduce and/or ameliorate the symptoms and/or underlying causes of a disorder or disease, for example to prevent, inhibit, and/or treat EBOV infection. In some embodiments, a therapeutically effective amount is sufficient to reduce or eliminate a symptom of a disease, such as EVD. For instance, this can be the amount necessary to inhibit or prevent EBOV replication or to measurably alter outward symptoms of the EBOV infection. Ideally, a therapeutically effective amount provides a therapeutic effect without causing a substantial cytotoxic effect in the subject.

In some embodiments, a desired response is to inhibit or reduce or prevent EBOV infection. The EBOV infection does not need to be completely eliminated or reduced or prevented for the method to be effective. For example, administration of a therapeutically effective amount of the agent can reduce or inhibit the EBOV infection (for example, as measured by infection of cells, or by number or percentage of subjects infected by EBOV, or by an increase in the survival time of infected subjects) by a desired amount, for example by at least 10%, at least 20%, at least 50%, at least 60%, at least 70%, at least 80%, at least 90%, at least 95%, at least 98%, or even at least 100% (elimination or prevention of detectable EBOV infection, as compared to a suitable control).

A therapeutically effective amount of an antibody or antigen binding fragment that specifically binds EBOV GP that is administered to a subject will vary depending upon a number of factors associated with that subject, for example the overall health and/or weight of the subject. A therapeutically effective amount encompasses a fractional dose that contributes in combination with previous or subsequent administrations to attaining a therapeutic response. For example, a therapeutically effective amount of an agent can be administered in a single dose, or in several doses, for example daily, during a course of treatment lasting several days or weeks. However, the therapeutically effective amount can depend on the subject being treated, the severity and type of the condition being treated, and the manner of administration. A unit dosage form of the agent can be packaged in a therapeutic amount, or in multiples of the therapeutic amount, for example, in a vial (for example, with a pierceable lid) or syringe having sterile components.

Transformed: A transformed cell is a cell into which a nucleic acid molecule has been introduced by molecular biology techniques. As used herein, the term transformation encompasses all techniques by which a nucleic acid molecule might be introduced into such a cell, including transfection with viral vectors, transformation with plasmid vectors, and introduction of DNA by electroporation, lipofection, and particle gun acceleration.

Treating or preventing a disease: Inhibiting the full development of a disease or condition, for example, in a subject who is at risk of or has an EBOV infection. “Treatment” refers to a therapeutic intervention that ameliorates a sign or symptom of a disease or pathological condition after it has begun to develop. The term “ameliorating,” with reference to a disease or pathological condition, refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, a reduction in the viral load, an improvement in the overall health or well-being of the subject, or by other parameters well known in the art that are specific to the particular disease. A “prophylactic” treatment is a treatment administered to a subject who does not exhibit signs of a disease for the purpose of reducing the risk of developing pathology.

The term “reduces” is a relative term, such that an agent reduces a disease or condition (or a symptom of a disease or condition) if the disease or condition is quantitatively diminished following administration of the agent, or if it is diminished following administration of the agent, as compared to a reference agent. Similarly, the term “prevents” does not necessarily mean that an agent completely eliminates the disease or condition, so long as at least one characteristic of the disease or condition is eliminated. Thus, an antibody that reduces or prevents an infection, can, but does not necessarily completely, eliminate such an infection, so long as the infection is measurably diminished, for example, by at least about 50%, such as by at least about 70%, or about 80%, or even by about 90% the infection in the absence of the agent, or in comparison to a reference agent.

Under conditions sufficient for: A phrase that is used to describe any environment that permits a desired activity. In one example the desired activity is formation of an immune complex. In particular examples the desired activity is treatment of EBOV infection.

### II. Description of Several Embodiments

Isolated monoclonal antibodies and antigen binding fragments that specifically bind an epitope on EBOV GP protein are provided. The antibodies and antigen binding fragments can be fully human. In several embodiments, the antibodies and antigen binding fragments can be used to neutralize EBOV infection. Also disclosed herein are compositions including the antibodies and antigen binding fragments and a pharmaceutically acceptable carrier. Nucleic acids encoding the antibodies or antigen binding fragments, expression vectors including these nucleic acids, and isolated host cells that express the nucleic acids are also provided.

The antibodies, antigen binding fragments, nucleic acid molecules, host cells, and compositions can be used for research, diagnostic and therapeutic purposes. For example, the monoclonal antibodies and antigen binding fragments can be used to diagnose or treat a subject with an EBOV, or can be administered prophylactically to prevent EBOV infection in a subject. In some embodiments, the antibodies can be used to determine EBOV titer in a subject.

### A. Ebola Virus (EBOV)-Specific Monoclonal Antibodies

Eight human monoclonal antibodies that specifically bind EBOV GP with nanomolar affinity are described. The monoclonal antibodies were isolated by bulk sorting of plasmablasts from a human EBOV vaccinee and subsequent pairing of the immunoglobulin heavy and light chain genes using emulsion PCR. The paired immunoglobulin genes were expressed using Fab yeast display to enable screening and in vitro characterization of the antibodies (see Example 1). Four of the antibodies were tested for their capacity to neutralize EBOV and were found to have high potency (IC50 of 0.5 μg/ml to 7 μg/ml). The EBOV GP-specific monoclonal antibodies can be used, for example, to diagnose or treat EBOV infection or EVD in a subject.

Provided herein are monoclonal antibodies that bind EBOV GP. In some embodiments, the monoclonal antibodies include a variable heavy (VH) domain and/or a variable light (VL) domain, wherein the VH domain comprises the VH complementarity determining region (HCDR)1, HCDR2, and HCDR3 sequences of SEQ ID NO: 2 and/or the VL domain comprises the VL complementarity determining region (LCDR)1, LCDR2, and LCDR3 sequences of SEQ ID NO: 4; the VH domain comprises the HCDR1, HCDR2 and HCDR3 sequences of SEQ ID NO: 6 and/or the VL domain comprises the LCDR1, LCDR2 and LCDR3 sequences of SEQ ID NO: 8; the VH domain comprises the HCDR1, HCDR2 and HCDR3 sequences of SEQ ID NO: 10 and/or the VL domain comprises the LCDR1, LCDR2 and LCDR3 sequences of SEQ ID NO: 12; the VH domain comprises the HCDR1, HCDR2 and HCDR3 sequences of SEQ ID NO: 14 and/or the VL domain comprises the LCDR1, LCDR2 and LCDR3 sequences of SEQ ID NO: 16; the VH domain comprises the HCDR1, HCDR2 and HCDR3 sequences of SEQ ID NO: 18 and/or the VL domain comprises the LCDR1, LCDR2 and LCDR3 sequences of SEQ ID NO: 20; the VH domain comprises the HCDR1, HCDR2 and HCDR3 sequences of SEQ ID NO: 22 and/or the VL domain comprises the LCDR1, LCDR2 and LCDR3 sequences of SEQ ID NO: 24; the VH domain comprises the HCDR1, HCDR2 and HCDR3 sequences of SEQ ID NO: 26 and/or the VL domain comprises the LCDR1, LCDR2 and LCDR3 sequences of SEQ ID NO: 28; or the VH domain comprises the HCDR1, HCDR2 and HCDR3 sequences of SEQ ID NO: 30 and/or the VL domain comprises the LCDR1, LCDR2 and LCDR3 sequences of SEQ ID NO: 32. In some embodiments, the CDR sequences are determined using the IMGT, Kabat or Chothia numbering scheme.

In some embodiments, the monoclonal antibody neutralizes Ebola virus, for example ZEBOV. In some examples, the neutralization inhibitory concentration 50 (IC50) of the monoclonal antibody is less than 10 μg/ml, such as less than 8 μg/ml, less than 6 μg/ml, less than 5 μg/ml, less than 4 μg/ml, less than 3 μg/ml, less than 2 μg/ml or less than 1 μg/ml. In some examples, the Ebola virus is Zaire Ebola virus, Sudan Ebola virus, Tai Forest Ebola virus, or Bundibugyo Ebola virus.

In some examples, the HCDR1, HCDR2, and HCDR3 are respectively set forth as residues 26-33, 51-58 and 96-119 of SEQ ID NO: 2 and/or the LCDR1, LCDR2, and LCDR3 are respectively set forth as residues 27-37, 50-52 and 88-98 of SEQ ID NO: 4; the HCDR1, HCDR2, and HCDR3 are respectively set forth as residues 26-33, 51-58 and 96-113 of SEQ ID NO: 6 and/or the LCDR1, LCDR2, and LCDR3 are respectively set forth as residues 27-32, 50-52 and 89-97 of SEQ ID NO: 8; the HCDR1, HCDR2, and HCDR3 are respectively set forth as residues 26-33, 51-57 and 95-116 of SEQ ID NO: 10 and/or the LCDR1, LCDR2, and LCDR3 are respectively set forth as residues 26-31, 49-51 and 87-97 of SEQ ID NO: 12; the HCDR1, HCDR2, and HCDR3 are respectively set forth as residues 26-33, 51-60 and 98-113 of SEQ ID NO: 14 and/or the LCDR1, LCDR2, and LCDR3 are respectively set forth as residues 26-34, 52-54 and 90-103 of SEQ ID NO: 16; the HCDR1, HCDR2, and HCDR3 are respectively set forth as residues 26-33, 51-73 and 111-126 of SEQ ID NO: 18 and/or the LCDR1, LCDR2, and LCDR3 are respectively set forth as residues 27-32, 50-52 and 88-98 of SEQ ID NO: 20; the HCDR1, HCDR2, and HCDR3 are respectively set forth as residues 27-35, 53-59 and 97-122 of SEQ ID NO: 22 and/or the LCDR1, LCDR2, and LCDR3 are respectively set forth as residues 27-32, 50-52 and 88-99 of SEQ ID NO: 24; the HCDR1, HCDR2, and HCDR3 are respectively set forth as residues 27-33, 51-60 and 98-109 of SEQ ID NO: 26 and/or the LCDR1, LCDR2, and LCDR3 are respectively set forth as residues 26-34, 52-54 and 90-103 of SEQ ID NO: 28; or the HCDR1, HCDR2, and HCDR3 are respectively set forth as residues 26-33, 51-58 and 96-115 of SEQ ID NO: 30 and/or the LCDR1, LCDR2, and LCDR3 are respectively set forth as residues 25-33, 51-53 and 89-101 of SEQ ID NO: 32.

In some examples, the amino acid sequence of the VH domain is at least 90% identical to SEQ ID NO: 2 and/or the amino acid sequence of the VL domain is at least 90% identical to SEQ ID NO: 4; the amino acid sequence of the VH domain is at least 90% identical to SEQ ID NO: 6 and/or the amino acid sequence of the VL domain is at least 90% identical to SEQ ID NO: 8; the amino acid sequence of the VH domain is at least 90% identical to SEQ ID NO: 10 and/or the amino acid sequence of the VL domain is at least 90% identical to SEQ ID NO: 12; the amino acid sequence of the VH domain is at least 90% identical to SEQ ID NO: 14 and/or the amino acid sequence of the VL domain is at least 90% identical to SEQ ID NO: 16; the amino acid sequence of the VH domain is at least 90% identical to SEQ ID NO: 18 and/or the amino acid sequence of the VL domain is at least 90% identical to SEQ ID NO: 20; the amino acid sequence of the VH domain is at least 90% identical to SEQ ID NO: 22 and/or the amino acid sequence of the VL domain is at least 90% identical to SEQ ID NO: 24; the amino acid sequence of the VH domain is at least 90% identical to SEQ ID NO: 26 and/or the amino acid sequence of the VL domain is at least 90% identical to SEQ ID NO: 28; or the amino acid sequence of the VH domain is at least 90% identical to SEQ ID NO: 30 and/or the amino acid sequence of the VL domain is at least 90% identical to SEQ ID NO: 32.

In specific non-limiting examples, the amino acid sequence of the VH domain comprises SEQ ID NO: 2 and/or the amino acid sequence of the VL domain comprises SEQ ID NO: 4; the amino acid sequence of the VH domain comprises SEQ ID NO: 6 and/or the amino acid sequence of the VL domain comprises SEQ ID NO: 8; the amino acid sequence of the VH domain comprises SEQ ID NO: 10 and/or the amino acid sequence of the VL domain comprises SEQ ID NO: 12; the amino acid sequence of the VH domain comprises SEQ ID NO: 14 and/or the amino acid sequence of the VL domain comprises SEQ ID NO: 16; the amino acid sequence of the VH domain comprises SEQ ID NO: 18 and/or the amino acid sequence of the VL domain comprises SEQ ID NO: 20; the amino acid sequence of the VH domain comprises SEQ ID NO: 22 and/or the amino acid sequence of the VL domain comprises SEQ ID NO: 24; the amino acid sequence of the VH domain comprises SEQ ID NO: 26 and/or the amino acid sequence of the VL domain comprises SEQ ID NO: 28; or the amino acid sequence of the VH domain comprises SEQ ID NO: 30 and/or the amino acid sequence of the VL domain comprises SEQ ID NO: 32.

In some embodiments, the monoclonal antibody is an IgG, IgM or IgA. In some examples, the IgG is IgG1. In other examples, the IgG is IgG2, IgG3 or IgG4.

In some embodiments, the monoclonal antibody comprises a human constant region. In other embodiments, the monoclonal antibody comprises a non-human constant region, such as a murine constant region, a goal constant region, or a rabbit constant region.

In some embodiments, the constant region of the monoclonal antibody includes one or more amino acid substitutions to optimize in vivo half-life of the antibody. The serum half-life of IgG antibodies is regulated by the neonatal Fc receptor (FcRn). Thus, in several embodiments, the antibody includes an amino acid substitution that increases binding to the FcRn. Several such substitutions are known to the person of ordinary skill in the art, such as substitutions at IgG constant regions T250Q and M428L (see, e.g., Hinton et al., J Immunol., 176:346-356, 2006); M428L and N434S (the “LS” mutation, see, e.g., Zalevsky, et al., Nature Biotechnology, 28:157-159, 2010); N434A (see, e.g., Petkova et al., Int. Immunol., 18:1759-1769, 2006); T307A, E380A, and N434A (see, e.g., Petkova et al., Int. Immunol., 18:1759-1769, 2006); and M252Y, S254T, and T256E (see, e.g., Dall'Acqua et al., J. Biol. Chem., 281:23514-23524, 2006). The disclosed antibodies and antigen binding fragments can be linked to a Fc polypeptide including any of the substitutions listed above, for example, the Fc polypeptide comprises the M428L and N434S substitutions.

Also provided are antigen-binding fragments of a monoclonal antibody disclosed herein.

In some embodiments, the antigen-binding fragment is an Fab fragment, an Fab′ fragment, an F(ab)′₂ fragment, a single chain variable fragment (scFv) or a disulfide stabilized variable fragment (dsFv).

In some embodiments, the monoclonal antibody or antigen-binding fragment comprises a human framework region.

In some embodiments, the monoclonal antibody or antigen-binding fragment is a fully human antibody or antigen-binding fragment.

In some embodiments, the monoclonal antibody or antigen binding fragment is linked to an effector molecule or a detectable marker. In some examples, the detectable marker is a fluorescent, enzymatic, or radioactive marker.

Also provided herein are monoclonal antibodies and antigen-binding fragments that bind to the same epitope as a monoclonal antibody or antigen-binding fragment disclosed herein, wherein the monoclonal antibody or antigen binding fragment thereof neutralizes Ebola virus. In some examples, provided herein are monoclonal antibodies and antigen-binding fragments that bind to the same epitope as one or more of EboV.YD.01, EboV.YD.02, EboV.YD.03, EboV.YD.04, EboV.YD.05, EboV.YD.06, EboV.YD.07, and EboV.YD.08, and neutralize EOBV.

Further provided herein are isolated nucleic acid molecule that encode the VH domain and/or the VL domain of a monoclonal antibody or antigen-binding fragment disclosed herein. In some embodiments, the nucleic acid molecule is a recombinant nucleic acid molecule. In some embodiments, the nucleic acid comprises a cDNA molecule encoding the monoclonal antibody or antigen binding fragment. In some examples, the VH domain and/or the VL domain of the monoclonal antibody or antigen binding fragment comprise the nucleic acid sequences set forth as: SEQ ID NOs: 1 and 3, respectively, or degenerate variants thereof; SEQ ID NOs: 5 and 7, respectively, or degenerate variants thereof; SEQ ID NOs: 9 and 11, respectively, or degenerate variants thereof; SEQ ID NOs: 13 and 15, respectively, or degenerate variants thereof; SEQ ID NOs: 17 and 19, respectively, or degenerate variants thereof; SEQ ID NOs: 21 and 23, respectively, or degenerate variants thereof; SEQ ID NOs: 25 and 27, respectively, or degenerate variants thereof; or SEQ ID NOs: 29 and 31, respectively, or degenerate variants thereof. In some examples, the nucleic acid molecule is operably linked to a promoter.

Expression vectors that include a nucleic acid molecule disclosed herein are also provided, as are isolated cells that include an expression vector.

Also provided are pharmaceutical compositions, such as for use in treating or inhibiting an Ebola virus infection. In some embodiments, the pharmaceutical composition includes a therapeutically effective amount of a monoclonal antibody, antigen binding fragment, nucleic acid molecule, or expression vector disclosed herein; and a pharmaceutically acceptable carrier. In some examples, the composition is sterile and/or is in unit dosage form or a multiple thereof. In some examples, the Ebola virus is Ebola virus Zaire. In other examples, the Ebola virus is Sudan Ebola virus, Tai Forest Ebola virus, or Bundibugyo Ebola virus.

Further provided is a method of detecting an Ebola virus infection in a subject. In some embodiments, the method includes contacting a biological sample from the subject with the monoclonal antibody or antigen binding fragment disclosed herein under conditions sufficient to form an immune complex; and detecting the presence of the immune complex in the sample, wherein the presence of the immune complex in the sample indicates that the subject has the Ebola virus infection. In some examples, the Ebola virus is Ebola virus Zaire. In some examples, the Ebola virus is Sudan Ebola virus, Tai Forest Ebola virus, or Bundibugyo Ebola virus.

Also provided is a method of preventing or treating an Ebola virus infection in a subject. In some embodiments, the method includes administering to the subject a therapeutically effective amount of an antibody, antigen binding fragment, nucleic acid molecule, expression vector, or pharmaceutical composition disclosed herein. In some examples, the method further includes administering to the subject one or more additional antibodies or antigen binding fragments that specifically bind to Ebola virus GP and neutralize Ebola virus, or one or more nucleic acid molecules encoding the additional antibodies or antigen binding fragments. In some examples, the Ebola virus is Ebola virus Zaire. In some examples, the Ebola virus is Sudan Ebola virus, Tai Forest Ebola virus, or Bundibugyo Ebola virus.

Further provided is a method of producing a monoclonal antibody or antigen binding fragment that specifically binds to Ebola virus GP. In some embodiments, the method includes expressing first and second nucleic acid molecules encoding the VH domain and the VL domain, respectively, of a monoclonal antibody or antigen binding fragment disclosed herein in a host cell, or expressing a nucleic acid molecule encoding the VH domain and the VL domain of the monoclonal antibody or antigen binding fragment disclosed herein in the host cell; and purifying the antibody or antigen binding fragment.

Use of a monoclonal antibody, antigen binding fragment, nucleic acid molecule, expression vector, or pharmaceutical composition disclosed herein to treat, prevent, or diagnose Ebola virus infection in a subject is further provided.

1. Additional Description of Antibodies and Antigen-Binding Fragments

The antibody or antigen binding fragment can be a human antibody or fragment thereof. Chimeric antibodies are also provided. The antibody or antigen binding fragment can include any suitable framework region, such as (but not limited to) a human framework region. Human framework regions, and mutations that can be made in a human antibody framework regions, are known in the art (see, for example, in U.S. Pat. No. 5,585,089, which is incorporated herein by reference). Alternatively, a heterologous framework region, such as, but not limited to a mouse or monkey framework region, can be included in the heavy or light chain of the antibodies (see, for example, Jones et al., Nature 321:522, 1986; Riechmann et al., Nature 332:323, 1988; Verhoeyen et al., Science 239:1534, 1988; Carter et al., Proc. Natl. Acad. Sci. U.S.A. 89:4285, 1992; Sandhu, Crit. Rev. Biotech. 12:437, 1992; and Singer et al., J. Immunol. 150:2844, 1993.)

The antibody can be of any isotype. The antibody can be, for example, an IgM or an IgG antibody, such as IgG₁, IgG₂, IgG₃, or IgG₄. The class of an antibody that specifically binds EBOV GP can be switched with another. In one aspect, a nucleic acid molecule encoding V_{L} or V_{H} is isolated using methods well-known in the art, such that it does not include any nucleic acid sequences encoding the constant region of the light or heavy chain, respectively. A nucleic acid molecule encoding V_{L} or V_{H} is then operatively linked to a nucleic acid sequence encoding a C_{L} or C_{H} from a different class of immunoglobulin molecule. This can be achieved using a vector or nucleic acid molecule that comprises a C_{L} or C_{H} chain, as known in the art. For example, an antibody that specifically binds EBOV GP, that was originally IgM may be class switched to an IgG. Class switching can be used to convert one IgG subclass to another, such as from IgG₁ to IgG₂, IgG₃, or IgG₄.

In some examples, the disclosed antibodies are oligomers of antibodies, such as dimers, trimers, tetramers, pentamers, hexamers, septamers, octomers and so on.

(a) Binding Affinity

In several embodiments, the antibody or antigen binding fragment can specifically bind EBOV GP with an affinity (for example, measured by K_{d}) of no more than 1.0×10⁻⁶M, no more than 5.0×10⁻⁶M, no more than 1.0×10⁻⁷M, no more than 5.0×10⁻⁷M, no more than 1.0×10⁻⁸M, no more than 5.0×10⁻⁸M, or no more than 1.0×10⁻⁹M, no more than 5.0×10⁻⁹M, no more than 1.0×10⁻¹° M, no more than 5.0×10⁻¹⁰ M, or no more than 1.0×10⁻¹¹M, no more than 5.0×10⁻¹¹M, or no more than 1.0×10⁻¹²M. K_{d} can be measured, for example, by a radiolabeled antigen binding assay (RIA) performed with the Fab version of an antibody of interest and its antigen using known methods. In one assay, solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, for example, Chen et al., J. Mol. Biol. 293:865-881, 1999, which is incorporated by reference herein in its entirety). To establish conditions for the assay, MICROTITER® multi-well plates (Thermo Scientific) are coated overnight with 5 μg/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620), 100 μM or 26 pM [¹²⁵I]-antigen are mixed with serial dilutions of a Fab of interest (for example, consistent with assessment of the anti-VEGF antibody, Fab-12, in Presta et al., Cancer Res. 57:4593-4599 (1997)). The Fab of interest is then incubated overnight; however, the incubation may continue for a longer period (for example, about 65 hours) to ensure that equilibrium is reached. Thereafter, the mixtures are transferred to the capture plate for incubation at room temperature (such as for one hour). The solution is then removed and the plate washed eight times with 0.1% polysorbate 20 (TWEEN-20®) in PBS. When the plates have dried, 150 μl/well of scintillant (MICROSCINT-20™; Packard) is added, and the plates are counted on a TOPCOUNT™ gamma counter (Packard) for ten minutes. Concentrations of each Fab that give less than or equal to 20% of maximal binding are chosen for use in competitive binding assays.

In another assay, K_{d} can be measured using surface plasmon resonance assays using a BIACORE®-2000 or a BIACORE®-3000 (BIAcore, Inc., Piscataway, N.J.) at 25° C. with immobilized antigen CM5 chips at ˜10 response units (RU). Briefly, carboxymethylated dextran biosensor chips (CM5, BIACORE®, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (˜0.2 μM) before injection at a flow rate of five 1/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25° C. at a flow rate of approximately 25 l/min. Association rates (kₒₙ) and dissociation rates (k_{off}) are calculated using a simple one-to-one Langmuir binding model (BIACORE® Evaluation Software version 3.2) by simultaneously fitting the association and dissociation sensorgrams. The equilibrium dissociation constant (Kd) is calculated as the ratio k_{off}/kₒₙ (see, for example, Chen et al., J. Mol. Biol. 293:865-881 (1999). If the on-rate exceeds 10⁶ M⁻¹s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophotometer (Aviv Instruments) or a 8000-series SLM-AMINCO™ spectrophotometer (ThermoSpectronic) with a stirred cuvette.

K_{d} can also be measured using biolayer interferometry, as described in Example 1 (see also, Misasi et al., Science 351, 1343-1346, 2016).

(b) Neutralization

In several embodiments, the antibodies and antigen binding fragments disclosed herein can neutralize EBOV infection by at least two, at least three, at least four, or at least five strains of EBOV, such as the Bundibugyo (BDBV), Reston (RESTV), Sudan (SUDV), Taï Forest (TAFV), and Zaire (ZEBOV), with an IC50 of less than 50 μg/ml. In more embodiments, the antibodies and antigen binding fragments disclosed herein can neutralize EBOV infection by at least two, at least three, at least four, or at least five strains of EBOV, such as the BDBV, RESTV, SUDV, TAFV, and ZEBOV, with an IC50 of less than 10 μg/ml. In several embodiments the antibodies and antigen binding fragments disclosed herein can neutralize infection by ZEBOV, with an IC50 of less than 50 μg/ml or less than 10 μg/ml. An exemplary method of assaying EBOV neutralization is provided in the Examples. In some embodiments, neutralization assays can be performed using a single-round EBOV GP-pseudoviruses infection of 293-T cells. In some embodiments, methods to assay for neutralization activity includes a single-cycle infection assay as described in Martin et al. (2003) Nature Biotechnology 21:71-76. In this assay, the level of viral activity is measured via a selectable marker whose activity is reflective of the amount of viable virus in the sample, and the IC₅₀ is determined.

(c) Multispecific Antibodies

In some embodiments, the antibody or antigen binding fragment is included on a multispecific antibody, such as a bi-specific antibody. Such multispecific antibodies can be produced by known methods, such as crosslinking two or more antibodies, or antigen binding fragments (such as scFvs) of the same type or of different types. Exemplary methods of making multispecific antibodies include those described in PCT Pub. No. WO 2013/163427, which is incorporated by reference herein in its entirety. Suitable crosslinkers include those that are heterobifunctional, having two distinctly reactive groups separated by an appropriate spacer (such as m-maleimidobenzoyl-N-hydroxysuccinimide ester) or homobifunctional (such as disuccinimidyl suberate). Such linkers are available from Pierce Chemical Company, Rockford, Ill.

Various types of multi-specific antibodies are known. Bispecific single chain antibodies can be encoded by a single nucleic acid molecule. Examples of bispecific single chain antibodies, as well as methods of constructing such antibodies are known in the art (see, e.g., U.S. Pat. Nos. 8,076,459, 8,017,748, 8,007,796, 7,919,089, 7,820,166, 7,635,472, 7,575,923, 7,435,549, 7,332,168, 7,323,440, 7,235,641, 7,229,760, 7,112,324, 6,723,538, incorporated by reference herein). Additional examples of bispecific single chain antibodies can be found in PCT application No. WO 99/54440; Mack, J. Immunol., 158:3965-3970, 1997; Mack, PNAS, 92:7021-7025, 1995; Kufer, Cancer Immunol. Immunother., 45:193-197, 1997; Loffler, Blood, 95:2098-2103, 2000; and Bruhl, J. Immunol., 166:2420-2426, 2001. Production of bispecific Fab-scFv (“bibody”) molecules are described, for example, in Schoonjans et al. (J. Immunol. 165:7050-57, 2000) and Willems et al. (J Chromatogr B Analyt Technol Biomed Life Sci. 786:161-76, 2003). For bibodies, a scFv molecule can be fused to one of the VL-CL (L) or VH-CH1 chains, e.g., to produce a bibody one scFv is fused to the C-terminus of a Fab chain.

(d) Fragments

Antigen binding fragments are encompassed by the present disclosure, such as Fab, F(ab′)₂, and Fv which include a heavy chain and light chain variable region and specifically bind EBOV GP. These antibody fragments retain the ability to selectively bind with the antigen and are “antigen-binding” fragments. Non-limiting examples of such fragments include:

(1) Fab, the fragment which contains a monovalent antigen-binding fragment of an antibody molecule, can be produced by digestion of whole antibody with the enzyme papain to yield an intact light chain and a portion of one heavy chain;

(2) Fab′, the fragment of an antibody molecule can be obtained by treating whole antibody with pepsin, followed by reduction, to yield an intact light chain and a portion of the heavy chain; two Fab′ fragments are obtained per antibody molecule;

(3) (Fab′)₂, the fragment of the antibody that can be obtained by treating whole antibody with the enzyme pepsin without subsequent reduction; F(ab′)₂ is a dimer of two Fab′ fragments held together by two disulfide bonds;

(4) Fv, a genetically engineered fragment containing the V_{H} and V_{L} expressed as two chains; and

(5) Single chain antibody (such as scFv), defined as a genetically engineered molecule containing the variable region of the light chain, the variable region of the heavy chain, linked by a suitable polypeptide linker as a genetically fused single chain molecule. A scFv is a fusion protein in which a V_{L} of an immunoglobulin and a VH of an immunoglobulin are bound by a linker (see, for example, Ahmad et al., Clin. Dev. Immunol., 2012: 980250, 2012; Mabry and Snavely, IDrugs, 13:543-549, 2010). The intramolecular orientation of the V_{H}-domain and the V_{L}-domain in a scFv, is not decisive for the provided antibodies (for example, for the provided multispecific antibodies). Thus, scFvs with both possible arrangements (V_{H} domain-linker domain-V_{L} domain; V_{L} domain-linker domain-V_{H} domain) may be used.

(6) A dimer of a single chain antibody (scFv₂), defined as a dimer of a scFv. This has also been termed a “miniantibody.”

Methods of making these fragments are known in the art (see for example, Harlow and Lane, Antibodies: A Laboratory Manual, 2^{nd}, Cold Spring Harbor Laboratory, New York, 2013).

In some embodiments, the antigen binding fragment can be an Fv antibody, which is typically about 25 kDa and contain a complete antigen-binding site with three CDRs per each heavy chain and each light chain. To produce Fᵥ antibodies, the V_{H} and the V_{L} can be expressed from two individual nucleic acid constructs in a host cell.

If the V_{H} and the V_{L} are expressed non-contiguously, the chains of the Fv antibody are typically held together by noncovalent interactions. However, these chains tend to dissociate upon dilution, so methods have been developed to crosslink the chains through glutaraldehyde, intermolecular disulfides, or a peptide linker Thus, in one example, the Fv can be a disulfide stabilized Fv (dsFv), wherein the V_{H} and the V_{L} are chemically linked by disulfide bonds.

In an additional example, the Fv fragments comprise V_{H} and V_{L} chains connected by a peptide linker. These single-chain antigen binding proteins (scFv) are prepared by constructing a nucleic acid molecule encoding the V_{H} and V_{L} domains connected by an oligonucleotide. The nucleic acid molecule is inserted into an expression vector, which is subsequently introduced into a host cell such as a mammalian cell. The recombinant host cells synthesize a single polypeptide chain with a linker peptide bridging the two V domains. Methods for producing scFvs are known in the art (see Whitlow et al., Methods: a Companion to Methods in Enzymology, Vol. 2, page 97, 1991; Bird et al., Science 242:423, 1988; U.S. Pat. No. 4,946,778; Pack et al., Bio/Technology 11:1271, 1993; Ahmad et al., Clin. Dev. Immunol., 2012: 980250, 2012; Mabry and Snavely, IDrugs, 13:543-549, 2010). Dimers of a single chain antibody (scFV₂), are also contemplated.

Antigen binding fragments can be prepared by proteolytic hydrolysis of the antibody or by expression in a host cell (such as an E. coli cell) of DNA encoding the fragment. Antigen binding fragments can also be obtained by pepsin or papain digestion of whole antibodies by conventional methods. For example, antigen binding fragments can be produced by enzymatic cleavage of antibodies with pepsin to provide a 5S fragment denoted F(ab′)₂. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly (see U.S. Pat. Nos. 4,036,945 and 4,331,647, and references contained therein; Nisonhoff et al., Arch. Biochem. Biophys. 89:230, 1960; Porter, Biochem. J. 73:119, 1959; Edelman et al., Methods in Enzymology, Vol. 1, page 422, Academic Press, 1967; and Coligan et al. at sections 2.8.1-2.8.10 and 2.10.1-2.10.4).

Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

Antigen binding single V_{H} domains, called domain antibodies (dAb), have also been identified from a library of murine V_{H} genes amplified from genomic DNA of immunized mice (Ward et al. Nature 341:544-546, 1989). Human single immunoglobulin variable domain polypeptides capable of binding antigen with high affinity have also been described (see, for example, PCT Publication Nos. WO 2005/035572 and WO 2003/002609). The CDRs disclosed herein can also be included in a dAb.

In some embodiments, one or more of the heavy and/or light chain complementarity determining regions (CDRs) from a disclosed antibody is expressed on the surface of another protein, such as a scaffold protein. The expression of domains of antibodies on the surface of a scaffolding protein are known in the art (see, for example, Liu et al., J. Virology 85(17): 8467-8476, 2011). Such expression creates a chimeric protein that retains the binding for EBOV GP. In some specific embodiments, one or more of the heavy chain CDRs is grafted onto a scaffold protein, such as one or more of heavy chain CDR1, CDR2, and/or CDR3. One or more CDRs can also be included in a diabody or another type of single chain antibody molecule.

(e) Additional antibodies that bind to the EboV.YD.01, EboV.YD.02, EboV.YD.03, EboV.YD.04, EboV.YD.05, EboV.YD.06, EboV.YD.07 or EboV.YD.08 epitope on EBOV GP

Also included are antibodies that bind to the same epitope on EBOV GP to which the EboV.YD.01, EboV.YD.02, EboV.YD.03, EboV.YD.04, EboV.YD.05, EboV.YD.06, EboV.YD.07, or EboV.YD.08 antibody binds. Antibodies that bind to such an epitope can be identified based on their ability to cross-compete (for example, to competitively inhibit the binding of, in a statistically significant manner) with the EboV.YD.01, EboV.YD.02, EboV.YD.03, EboV.YD.04, EboV.YD.05, EboV.YD.06, EboV.YD.07, or EboV.YD.08 antibodies provided herein in EBOV GP binding assays (such as those described in the Examples). An antibody “competes” for binding when the competing antibody inhibits EBOV GP binding of the EboV.YD.01, EboV.YD.02, EboV.YD.03, EboV.YD.04, EboV.YD.05, EboV.YD.06, EboV.YD.07, or EboV.YD.08 antibody by more than 50%, in the presence of competing antibody concentrations higher than 10⁶×K_{D} of the competing antibody. In a certain embodiment, the antibody that binds to the same epitope on EBOV GP as the EboV.YD.01, EboV.YD.02, EboV.YD.03, EboV.YD.04, EboV.YD.05, EboV.YD.06, EboV.YD.07, or EboV.YD.08 antibody is a human monoclonal antibody. Human antibodies that bind to the same epitope on EBOV GP to which the EboV.YD.01, EboV.YD.02, EboV.YD.03, EboV.YD.04, EboV.YD.05, EboV.YD.06, EboV.YD.07, or EboV.YD.08 antibody binds can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008). Such antibodies may be prepared, for example, by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005) (see also, for example, U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, for example, by combining with a different human constant region.

Human antibodies that bind to the same epitope on EBOV GP to which the EboV.YD.01, EboV.YD.02, EboV.YD.03, EboV.YD.04, EboV.YD.05, EboV.YD.06, EboV.YD.07, or EboV.YD.08 antibody binds can also be made by hybridoma-based methods. Human myeloma and mouse-human heteromyeloma cell lines for the production of human monoclonal antibodies have been described (see, for example, Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3): 185-91 (2005). Human antibodies may also be generated by isolating Fv clone variable domain sequences selected from human-derived phage display libraries. Such variable domain sequences may then be combined with a desired human constant domain.

Antibodies and antigen binding fragments that specifically bind to the same epitope on EBOV GP as EboV.YD.01, EboV.YD.02, EboV.YD.03, EboV.YD.04, EboV.YD.05, EboV.YD.06, EboV.YD.07, or EboV.YD.08 can also be isolated by screening combinatorial libraries for antibodies with the desired binding characteristics. For example, a variety of methods are known in the art for generating phage display libraries and screening such libraries for antibodies possessing the desired binding characteristics. Such methods are reviewed, for example, in Hoogenboom et al. in Methods in Molecular Biology 178:1-37 (O'Brien et al., ed., Human Press, Totowa, N.J., 2001) and further described, for example, in the McCafferty et al., Nature 348:552-554; Clackson et al., Nature 352: 624-628 (1991); Marks et al., J. Mol. Biol. 222: 581-597 (1992); Marks and Bradbury, in Methods in Molecular Biology 248:161-175 (Lo, ed., Human Press, Totowa, N.J., 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132 (2004).

In certain phage display methods, repertoires of V_{H} and V_{L} genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naïve repertoire can be cloned (for example, from humans) to provide a single source of antibodies to a wide range of non-self and also self-antigens without any immunization as described by Griffiths et al., EMBO J, 12: 725-734 (1993). Finally, naïve libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

(f) Variants

In certain embodiments, amino acid sequence variants of the antibodies provided herein are contemplated. For example, it may be desirable to improve the binding affinity and/or other biological properties of the antibody Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, for example, antigen-binding.

In certain embodiments, antibody variants having one or more amino acid substitutions are provided. Sites of interest for substitutional mutagenesis include the CDRs and the framework regions. Amino acid substitutions may be introduced into an antibody of interest and the products screened for a desired activity, for example, retained/improved antigen binding, decreased immunogenicity, or improved ADCC or CDC.

The variants typically retain amino acid residues necessary for correct folding and stabilizing between the V_{H} and the V_{L} regions, and will retain the charge characteristics of the residues in order to preserve the low pI and low toxicity of the molecules Amino acid substitutions can be made in the V_{H} and the V_{L} regions to increase yield.

In some embodiments, the heavy chain of the antibody includes up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NOs: 2, 6, 10, 14, 18, 22, 26 or 30. In some embodiments, the light chain of the antibody includes up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NOs: 4, 8, 12, 16, 20, 24, 28 or 32.

In some embodiments, the antibody or antigen binding fragment can include up to 10 (such as up to 1, up to 2, up to 3, up to 4, up to 5, up to 6, up to 7, up to 8, or up to 9) amino acid substitutions (such as conservative amino acid substitutions) in the framework regions of the heavy chain of the antibody, or the light chain of the antibody, or the heavy and light chains of the antibody, compared to a known framework region, or compared to the framework regions of the EboV.YD.01, EboV.YD.02, EboV.YD.03, EboV.YD.04, EboV.YD.05, EboV.YD.06, EboV.YD.07, or EboV.YD.08 antibody, and maintain the specific binding activity for EBOV GP.

In certain embodiments, substitutions, insertions, or deletions may occur within one or more CDRs so long as such alterations do not substantially reduce the ability of the antibody to bind antigen. For example, conservative alterations (such as conservative substitutions as provided herein) that do not substantially reduce binding affinity may be made in CDRs. In certain embodiments of the variant V_{H} and V_{L} sequences provided above, each CDR either is unaltered, or contains no more than one, two or three amino acid substitutions.

To increase binding affinity of the antibody, the V_{L} and V_{H} segments can be randomly mutated, such as within HCDR3 region or the LCDR3 region, in a process analogous to the in vivo somatic mutation process responsible for affinity maturation of antibodies during a natural immune response. Thus in vitro affinity maturation can be accomplished by amplifying V_{H} and V_{L} regions using PCR primers complementary to the HCDR3 or LCDR3, respectively. In this process, the primers have been “spiked” with a random mixture of the four nucleotide bases at certain positions such that the resultant PCR products encode V_{H} and V_{L} segments into which random mutations have been introduced into the V_{H} and/or V_{L} CDR3 regions. These randomly mutated V_{H} and V_{L} segments can be tested to determine the binding affinity for EBOV GP. In particular examples, the V_{H} amino acid sequence is one of SEQ ID NOs: 2, 6, 10, 14, 18, 22, 26 or 30. In other examples, the V_{L} amino acid sequence is one of SEQ ID NOs: 4, 8, 12, 16, 20, 24, 28 or 32. Methods of in vitro affinity maturation are known (see, for example, Chowdhury, Methods Mol. Biol. 207:179-196 (2008)), and Hoogenboom et al. in Methods in Molecular Biology 178:1-37 (O'Brien et al., ed., Human Press, Totowa, N.J., (2001).)

In certain embodiments, an antibody or antigen binding fragment is altered to increase or decrease the extent to which the antibody or antigen binding fragment is glycosylated. Addition or deletion of glycosylation sites may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH₂ domain of the Fc region (see, for example, Wright et al., TIBTECH 15:26-32, 1997). The oligosaccharide may include various carbohydrates, for example, mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody may be made in order to create antibody variants with certain improved properties.

In one embodiment, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (for example, complex, hybrid and high mannose structures) as measured by MALDI-TOF mass spectrometry, as described in WO 2008/077546, for example. Asn297 refers to the asparagine residue located at about position 297 in the Fc region; however, Asn297 may also be located about ±3 amino acids upstream or downstream of position 297, such as between positions 294 and 300, due to minor sequence variations in antibodies. Such fucosylation variants may have improved ADCC function (see, for example, US Patent Publication Nos. US 2003/0157108 and US 2004/0093621). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); and Yamane-Ohnuki et al. Biotech. Bioeng. 87: 614 (2004). Examples of cell lines capable of producing defucosylated antibodies include Lec 13 CHO cells deficient in protein fucosylation (Ripka et al. Arch. Biochem. Biophys. 249:533-545 (1986); US Pat Application No. US 2003/0157108; and WO 2004/056312, especially at Example 11), and knockout cell lines, such as alpha-1,6-fucosyltransferase gene, FUT8, knockout CHO cells (see, for example, Yamane-Ohnuki et al. Biotech. Bioeng. 87: 614 (2004); Kanda et al., Biotechnol. Bioeng., 94(4):680-688 (2006); and WO2003/085107).

Antibody variants are further provided with bisected oligosaccharides, for example, in which a biantennary oligosaccharide attached to the Fc region of the antibody is bisected by GlcNAc. Such antibody variants may have reduced fucosylation and/or improved ADCC function. Examples of such antibody variants are described, for example, in WO 2003/011878; U.S. Pat. No. 6,602,684; and US 2005/0123546. Antibody variants with at least one galactose residue in the oligosaccharide attached to the Fc region are also provided. Such antibody variants may have improved CDC function. Such antibody variants are described, for example, in WO 1997/30087; WO 1998/58964; and WO 1999/22764.

In several embodiments, the constant region of the antibody includes one or more amino acid substitutions to optimize in vivo half-life of the antibody. The serum half-life of IgG Abs is regulated by the neonatal Fc receptor (FcRn). Thus, in several embodiments, the antibody includes an amino acid substitution that increases binding to the FcRn. Several such substitutions are known, such as substitutions at IgG constant regions T250Q and M428L (see, for example, Hinton et al., J Immunol., 176:346-356, 2006); M428L and N434S (the “LS” mutation, see, for example, Zalevsky, et al., Nature Biotechnology, 28:157-159, 2010); N434A (see, for example, Petkova et al., Int. Immunol., 18:1759-1769, 2006); T307A, E380A, and N434A (see, for example, Petkova et al., Int. Immunol., 18:1759-1769, 2006); and M252Y, S254T, and T256E (see, for example, Dall'Acqua et al., J. Biol. Chem., 281:23514-23524, 2006). The disclosed antibodies and antigen binding fragments can be linked to a Fc polypeptide including any of the substitutions listed above, for example, the Fc polypeptide can include the M428L and N434S substitutions.

In some embodiments, the constant region of the antibody includes one of more amino acid substitutions to optimize antibody-dependent cell-mediated cytotoxicity (ADCC). ADCC is mediated primarily through a set of closely related Fcγ receptors. In some embodiments, the antibody includes one or more amino acid substitutions that increase binding to FcγRIIIa. Several such substitutions are known, such as substitutions at IgG constant regions S239D and I332E (see, for example, Lazar et al., Proc. Natl., Acad. Sci. U.S.A., 103:4005-4010, 2006); and S239D, A330L, and I332E (see, for example, Lazar et al., Proc. Natl., Acad. Sci. U.S.A., 103:4005-4010, 2006).

Combinations of the above substitutions are also included, to generate an IgG constant region with increased binding to FcRn and FcγRIIIa. The combinations increase antibody half-life and ADCC. For example, such combination include antibodies with the following amino acid substitutions in the Fc region:
(1) S239D/I332E and T250Q/M428L;
(2) S239D/I332E and M428L/N434S;
(3) S239D/I332E and N434A;
(4) S239D/I332E and T307A/E380A/N434A;
(5) S239D/I332E and M252Y/S254T/T256E;
(6) S239D/A330L/I332E and T250Q/M428L;
(7) S239D/A330L/I332E and M428L/N434S;
(8) S239D/A330L/I332E and N434A;
(9) S239D/A330L/I332E and T307A/E380A/N434A; or
(10) S239D/A330L/I332E and M252Y/S254T/T256E.

In some examples, the antibodies, or an antigen binding fragment thereof is modified such that it is directly cytotoxic to infected cells, or uses natural defenses such as complement, ADCC, or phagocytosis by macrophages.

In certain embodiments, an antibody provided herein may be further modified to contain additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (such as glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

The antibody or antigen binding fragment can be derivatized or linked to another molecule (such as another peptide or protein). In general, the antibody or antigen binding fragment is derivatized such that the binding to EBOV GP is not affected adversely by the derivatization or labeling. For example, the antibody or antigen binding fragment can be functionally linked (by chemical coupling, genetic fusion, noncovalent association or otherwise) to one or more other molecular entities, such as another antibody (for example, a bi-specific antibody or a diabody), a detectable marker, an effector molecule, or a protein or peptide that can mediate association of the antibody or antibody portion with another molecule (such as a streptavidin core region or a polyhistidine tag).

### B. Conjugates

The monoclonal antibodies and antigen binding fragments that specifically bind to an epitope on EBOV GP can be conjugated to an agent, such as an effector molecule or detectable label, using any number of means known to those of skill in the art. Both covalent and noncovalent attachment means may be used. One of skill in the art will appreciate that various effector molecules and detectable markers can be used, including (but not limited to) toxins and radioactive agents such as ¹²⁵I, ³²P, ¹⁴C, ³H and ³⁵S and other labels, target moieties and ligands, etc. The choice of a particular effector molecule or detectable marker depends on the particular target molecule or cell, and the desired biological effect.

The choice of a particular effector molecule or detectable marker depends on the particular target molecule or cell, and the desired biological effect. Thus, for example, the effector molecule can be a cytotoxin that is used to bring about the death of a particular target cell (such as an EBOV infected cell). In other embodiments, the effector molecule can be a cytokine, such as IL-15; conjugates including the cytokine can be used, for example, to stimulate immune cells locally.

The procedure for attaching an effector molecule or detectable marker to an antibody or antigen binding fragment varies according to the chemical structure of the effector. Polypeptides typically contain a variety of functional groups; such as carboxylic acid (COOH), free amine (—NH₂) or sulfhydryl (—SH) groups, which are available for reaction with a suitable functional group on a polypeptide to result in the binding of the effector molecule or detectable marker. Alternatively, the antibody or antigen binding fragment is derivatized to expose or attach additional reactive functional groups. The derivatization may involve attachment of any of a number of known linker molecules such as those available from Pierce Chemical Company, Rockford, Ill. The linker can be any molecule used to join the antibody or antigen binding fragment to the effector molecule or detectable marker. The linker is capable of forming covalent bonds to both the antibody/antigen binding fragment and to the effector molecule/detectable marker. Suitable linkers are well-known to those of skill in the art and include, but are not limited to, straight or branched-chain carbon linkers, heterocyclic carbon linkers, or peptide linkers. Where the antibody or antigen binding fragment and the effector molecule or detectable marker are polypeptides, the linkers may be joined to the constituent amino acids through their side groups (such as through a disulfide linkage to cysteine) or to the alpha carbon amino and carboxyl groups of the terminal amino acids.

In view of the large number of methods that have been reported for attaching a variety of radiodiagnostic compounds, radiotherapeutic compounds, labels (such as enzymes or fluorescent molecules), toxins, and other agents to antibodies one skilled in the art will be able to determine a suitable method for attaching a given agent to an antibody or antigen binding fragment or other polypeptide. For example, the antibody or antigen binding fragment can be conjugated with effector molecules such as small molecular weight drugs such as Monomethyl Auristatin E (MMAE), Monomethyl Auristatin F (MMAF), maytansine, maytansine derivatives, including the derivative of maytansine known as DM1 (also known as mertansine), or other agents to make an antibody drug conjugate (ADC). In several embodiments, conjugates of an antibody or antigen binding fragment and one or more small molecule toxins, such as a calicheamicin, maytansinoids, dolastatins, auristatins, a trichothecene, and CC1065, and the derivatives of these toxins that have toxin activity, are provided.

The antibody or antigen binding fragment can be conjugated with a detectable marker; for example, a detectable marker capable of detection by ELISA, spectrophotometry, flow cytometry, microscopy or diagnostic imaging techniques (such as computed tomography (CT), computed axial tomography (CAT) scans, magnetic resonance imaging (MRI), nuclear magnetic resonance imaging NMRI), magnetic resonance tomography (MTR), ultrasound, fiberoptic examination, and laparoscopic examination). Specific, non-limiting examples of detectable markers include fluorophores, chemiluminescent agents, enzymatic linkages, radioactive isotopes and heavy metals or compounds (for example super paramagnetic iron oxide nanocrystals for detection by MRI). For example, useful detectable markers include fluorescent compounds, including fluorescein, fluorescein isothiocyanate, rhodamine, 5-dimethylamine-1-napthalenesulfonyl chloride, phycoerythrin, lanthanide phosphors and the like. Bioluminescent markers are also of use, such as luciferase, green fluorescent protein (GFP), and yellow fluorescent protein (YFP). An antibody or antigen binding fragment can also be conjugated with enzymes that are useful for detection, such as horseradish peroxidase, β-galactosidase, luciferase, alkaline phosphatase, glucose oxidase and the like. When an antibody or antigen binding fragment is conjugated with a detectable enzyme, it can be detected by adding additional reagents that the enzyme uses to produce a reaction product that can be discerned. For example, when the agent horseradish peroxidase is present the addition of hydrogen peroxide and diaminobenzidine leads to a colored reaction product, which is visually detectable. An antibody or antigen binding fragment may also be conjugated with biotin, and detected through indirect measurement of avidin or streptavidin binding. It should be noted that the avidin itself can be conjugated with an enzyme or a fluorescent label.

The antibody or antigen binding fragment can be conjugated with a paramagnetic agent, such as gadolinium. Paramagnetic agents such as superparamagnetic iron oxide are also of use as labels. Antibodies can also be conjugated with lanthanides (such as europium and dysprosium), and manganese. An antibody or antigen binding fragment may also be labeled with a predetermined polypeptide epitopes recognized by a secondary reporter (such as leucine zipper pair sequences, binding sites for secondary antibodies, metal binding domains, epitope tags).

The antibody or antigen binding fragment can also be conjugated with a radiolabeled amino acid. The radiolabel may be used for both diagnostic and therapeutic purposes. For instance, the radiolabel may be used to detect EBOV GP and EBOV GP expressing cells by x-ray, emission spectra, or other diagnostic techniques. Examples of labels for polypeptides include, but are not limited to, the following radioisotopes or radionucleotides: ³H, ¹⁴C, ¹⁵N, ³⁵S, ⁹⁰Y, ⁹⁹Tc, ¹¹¹In, ¹²⁵I, ¹³¹I.

Means of detecting such detectable markers are well known to those of skill in the art. Thus, for example, radiolabels may be detected using photographic film or scintillation counters, fluorescent markers may be detected using a photodetector to detect emitted illumination. Enzymatic labels are typically detected by providing the enzyme with a substrate and detecting the reaction product produced by the action of the enzyme on the substrate, and colorimetric labels are detected by simply visualizing the colored label.

The average number of effector molecule or detectable marker moieties per antibody or antigen binding fragment in a conjugate can range, for example, from 1 to 20 moieties per antibody or antigen binding fragment. In certain embodiments, the average number of effector molecule or detectable marker moieties per antibody or antigen binding fragment in a conjugate range from about 1 to about 2, from about 1 to about 3, about 1 to about 8; from about 2 to about 6; from about 3 to about 5; or from about 3 to about 4. The loading (for example, effector molecule/antibody ratio) of an conjugate may be controlled in different ways, for example, by: (i) limiting the molar excess of effector molecule-linker intermediate or linker reagent relative to antibody, (ii) limiting the conjugation reaction time or temperature, (iii) partial or limiting reductive conditions for cysteine thiol modification, (iv) engineering by recombinant techniques the amino acid sequence of the antibody such that the number and position of cysteine residues is modified for control of the number or position of linker-effector molecule attachments.

### C. Polynucleotides and Expression

Nucleic acids molecules (for example, cDNA molecules) encoding the amino acid sequences of antibodies, antigen binding fragments, and conjugates that specifically bind EBOV GP are provided. Nucleic acids encoding these molecules can readily be produced by one of skill in the art, using the amino acid sequences provided herein (such as the CDR sequences and V_{H} and V_{L} sequences), sequences available in the art (such as framework or constant region sequences), and the genetic code. In several embodiments, a nucleic acid molecules can encode the V_{H}, the V_{L}, or both the V_{H} and V_{L} (for example in a bicistronic expression vector) of a disclosed antibody or antigen binding fragment. In several embodiments, the nucleic acid molecules can be expressed in a host cell (such as a mammalian cell) to produce a disclosed antibody or antigen binding fragment.

One of skill in the art can readily use the genetic code to construct a variety of functionally equivalent nucleic acids, such as nucleic acids which differ in sequence but which encode the same antibody sequence, or encode a conjugate or fusion protein including the V_{L} and/or V_{H} nucleic acid sequence.

In a non-limiting example, an isolated nucleic acid molecule encodes the V_{H} of a disclosed antibody or antigen binding fragment and includes the nucleic acid sequence set forth as any one of SEQ ID NOs: 1, 5, 9, 13, 17, 21, 25 or 29. In a non-limiting example, an isolated nucleic acid molecule encodes the V_{L} of a disclosed antibody or antigen binding fragment and includes the nucleic acid sequence set forth as any one of SEQ ID NOs: 3, 7, 11, 15, 19, 23, 27 or 31. In a non-limiting example, an isolated nucleic acid molecule encodes the V_{H} and V_{L} of a disclosed antibody or antigen binding fragment and includes the nucleic acid sequences set forth as any one of SEQ ID NOs: 1 and 3, respectively, 5 and 7, respectively, 9 and 11, respectively, 13 and 15, respectively, 17 and 19, respectively, 21 and 23, respectively, 25 and 27, respectively, or 29 and 31, respectively.

Nucleic acid sequences encoding antibodies, antigen binding fragments, and conjugates that specifically bind EBOV GP can be prepared by any suitable method including, for example, cloning of appropriate sequences or by direct chemical synthesis by methods such as the phosphotriester method of Narang et al., Meth. Enzymol. 68:90-99, 1979; the phosphodiester method of Brown et al., Meth. Enzymol. 68:109-151, 1979; the diethylphosphoramidite method of Beaucage et al., Tetra. Lett. 22:1859-1862, 1981; the solid phase phosphoramidite triester method described by Beaucage & Caruthers, Tetra. Letts. 22(20):1859-1862, 1981, for example, using an automated synthesizer as described in, for example, Needham-VanDevanter et al., Nucl. Acids Res. 12:6159-6168, 1984; and, the solid support method of U.S. Pat. No. 4,458,066. Chemical synthesis produces a single stranded oligonucleotide. This can be converted into double stranded DNA by hybridization with a complementary sequence or by polymerization with a DNA polymerase using the single strand as a template.

Exemplary nucleic acids can be prepared by cloning techniques. Examples of appropriate cloning and sequencing techniques, and instructions sufficient to direct persons of skill through many cloning exercises are known (see, for example, Sambrook et al. (Molecular Cloning: A Laboratory Manual, 4^{th} ed, Cold Spring Harbor, N.Y., 2012) and Ausubel et al. (In Current Protocols in Molecular Biology, John Wiley & Sons, New York, through supplement 104, 2013). Product information from manufacturers of biological reagents and experimental equipment also provide useful information. Such manufacturers include the SIGMA Chemical Company (Saint Louis, Mo.), R&D Systems (Minneapolis, Minn.), Pharmacia Amersham (Piscataway, N.J.), CLONTECH Laboratories, Inc. (Palo Alto, Calif.), Chem Genes Corp., Aldrich Chemical Company (Milwaukee, Wis.), Glen Research, Inc., GIBCO BRL Life Technologies, Inc. (Gaithersburg, Md.), Fluka Chemica-Biochemika Analytika (Fluka Chemie AG, Buchs, Switzerland), Invitrogen (Carlsbad, Calif.), and Applied Biosystems (Foster City, Calif.), as well as many other commercial sources known to one of skill.

Nucleic acids can also be prepared by amplification methods. Amplification methods include polymerase chain reaction (PCR), the ligase chain reaction (LCR), the transcription-based amplification system (TAS), the self-sustained sequence replication system (3SR). A wide variety of cloning methods, host cells, and in vitro amplification methodologies are well known to persons of skill.

The nucleic acid molecules can be expressed in a recombinantly engineered cell such as bacteria, plant, yeast, insect and mammalian cells. The antibodies, antigen binding fragments, and conjugates can be expressed as individual V_{H} and/or V_{L} chain (linked to an effector molecule or detectable marker as needed), or can be expressed as a fusion protein. Methods of expressing and purifying antibodies and antigen binding fragments are known and further described herein (see, for example, Al-Rubeai (ed), Antibody Expression and Production, Springer Press, 2011). An immunoadhesin can also be expressed. Thus, in some examples, nucleic acids encoding a V_{H} and V_{L}, and immunoadhesin are provided. The nucleic acid sequences can optionally encode a leader sequence.

To create a scFv the V_{H}- and V_{L}-encoding DNA fragments can be operatively linked to another fragment encoding a flexible linker, for example, encoding the amino acid sequence (Gly₄-Ser)₃, such that the V_{H} and V_{L} sequences can be expressed as a contiguous single-chain protein, with the V_{L} and V_{H} domains joined by the flexible linker (see, for example, Bird et al., Science 242:423-426, 1988; Huston et al., Proc. Natl. Acad. Sci. USA 85:5879-5883, 1988; McCafferty et al., Nature 348:552-554, 1990; Kontermann and Dubel (Ed), Antibody Engineering, Vols. 1-2, 2^{nd} Ed., Springer Press, 2010; Harlow and Lane, Antibodies: A Laboratory Manual, 2^{nd}, Cold Spring Harbor Laboratory, New York, 2013). Optionally, a cleavage site can be included in a linker, such as a furin cleavage site.

The nucleic acid encoding a V_{H} and/or the V_{L} optionally can encode an Fc domain. The Fc domain can be an IgA, IgM or IgG Fc domain. The Fc domain can be an optimized Fc domain, as described in U.S. Application Publication No. 20100/093979, incorporated herein by reference.

The single chain antibody may be monovalent, if only a single V_{H} and V_{L} are used, bivalent, if two V_{H} and V_{L} are used, or polyvalent, if more than two V_{H} and V_{L} are used. Bispecific or polyvalent antibodies may be generated that bind specifically to EBOV GP and another antigen. The encoded V_{H} and V_{L} optionally can include a furin cleavage site between the V_{H} and V_{L} domains.

Those of skill in the art are knowledgeable in the numerous expression systems available for expression of proteins including E. coli, other bacterial hosts, yeast, and various higher eukaryotic cells such as the COS, CHO, HeLa and myeloma cell lines.

One or more DNA sequences encoding the antibodies, antigen binding fragments, or conjugates can be expressed in vitro by DNA transfer into a suitable host cell. The cell may be prokaryotic or eukaryotic. The term also includes any progeny of the subject host cell. It is understood that all progeny may not be identical to the parental cell since there may be mutations that occur during replication. Methods of stable transfer, meaning that the foreign DNA is continuously maintained in the host, are known in the art. Hybridomas expressing the antibodies of interest are also encompassed by this disclosure.

The expression of nucleic acids encoding the antibodies and antigen binding fragments described herein can be achieved by operably linking the DNA or cDNA to a promoter (which is either constitutive or inducible), followed by incorporation into an expression cassette. The promoter can be any promoter of interest, including a cytomegalovirus promoter and a human T cell lymphotropic virus promoter (HTLV)-1. Optionally, an enhancer, such as a cytomegalovirus enhancer, is included in the construct. The cassettes can be suitable for replication and integration in either prokaryotes or eukaryotes. Typical expression cassettes contain specific sequences useful for regulation of the expression of the DNA encoding the protein. For example, the expression cassettes can include appropriate promoters, enhancers, transcription and translation terminators, initiation sequences, a start codon (ATG) in front of a protein-encoding gene, splicing signal for introns, sequences for the maintenance of the correct reading frame of that gene to permit proper translation of mRNA, and stop codons. The vector can encode a selectable marker, such as a marker encoding drug resistance (for example, ampicillin or tetracycline resistance).

To obtain high level expression of a cloned gene, it is desirable to construct expression cassettes which contain, at the minimum, a strong promoter to direct transcription, a ribosome binding site for translational initiation (internal ribosomal binding sequences), and a transcription/translation terminator. For E. coli, this includes a promoter such as the T7, trp, lac, or lambda promoters, a ribosome binding site, and preferably a transcription termination signal. For eukaryotic cells, the control sequences can include a promoter and/or an enhancer derived from, for example, an immunoglobulin gene, HTLV, SV40 or cytomegalovirus, and a polyadenylation sequence, and can further include splice donor and/or acceptor sequences (for example, CMV and/or HTLV splice acceptor and donor sequences). The cassettes can be transferred into the chosen host cell by well-known methods such as transformation or electroporation for E. coli and calcium phosphate treatment, electroporation or lipofection for mammalian cells. Cells transformed by the cassettes can be selected by resistance to antibiotics conferred by genes contained in the cassettes, such as the amp, gpt, neo and hyg genes.

When the host is a eukaryote, such methods of transfection of DNA as calcium phosphate coprecipitates, conventional mechanical procedures such as microinjection, electroporation, insertion of a plasmid encased in liposomes, or virus vectors may be used. Eukaryotic cells can also be cotransformed with polynucleotide sequences encoding the antibody, labeled antibody, or antigen biding fragment, and a second foreign DNA molecule encoding a selectable phenotype, such as the herpes simplex thymidine kinase gene. Another method is to use a eukaryotic viral vector, such as simian virus 40 (SV40) or bovine papilloma virus, to transiently infect or transform eukaryotic cells and express the protein (see for example, Viral Expression Vectors, Springer press, Muzyczka ed., 2011). One of skill in the art can readily use an expression systems such as plasmids and vectors of use in producing proteins in cells including higher eukaryotic cells such as the COS, CHO, HeLa and myeloma cell lines.

Also provided is a population of cells comprising at least one host cell described herein. The population of cells can be a heterogeneous population comprising the host cell comprising any of the recombinant expression vectors described, in addition to at least one other cell, for example, a host cell (such as a T cell), which does not comprise any of the recombinant expression vectors, or a cell other than a T cell, such as a B cell, a macrophage, a neutrophil, an erythrocyte, a hepatocyte, an endothelial cell, an epithelial cell, a muscle cell, a brain cell, etc. Alternatively, the population of cells can be a substantially homogeneous population, in which the population comprises mainly host cells (for example, consisting essentially of) comprising the recombinant expression vector. The population also can be a clonal population of cells, in which all cells of the population are clones of a single host cell comprising a recombinant expression vector, such that all cells of the population comprise the recombinant expression vector. In one embodiment, the population of cells is a clonal population comprising host cells comprising a recombinant expression vector as described herein

Modifications can be made to a nucleic acid encoding a polypeptide described herein without diminishing its biological activity. Some modifications can be made to facilitate the cloning, expression, or incorporation of the targeting molecule into a fusion protein. Such modifications are well known to those of skill in the art and include, for example, termination codons, a methionine added at the amino terminus to provide an initiation, site, additional amino acids placed on either terminus to create conveniently located restriction sites, or additional amino acids (such as poly His) to aid in purification steps. In addition to recombinant methods, the immunoconjugates, effector moieties, and antibodies of the present disclosure can also be constructed in whole or in part using standard peptide synthesis well known in the art.

Once expressed, the antibodies, antigen binding fragments, and conjugates can be purified according to standard procedures in the art, including ammonium sulfate precipitation, affinity columns, column chromatography, and the like (see, generally, Simpson ed., Basic methods in Protein Purification and Analysis: A laboratory Manual, Cold Harbor Press, 2008). The antibodies, antigen binding fragment, and conjugates need not be 100% pure. Once purified, partially or to homogeneity as desired, if to be used therapeutically, the polypeptides should be substantially free of endotoxin.

Methods for expression of the antibodies, antigen binding fragments, and conjugates, and/or refolding to an appropriate active form, from mammalian cells, and bacteria such as E. coli have been described and are well-known and are applicable to the antibodies disclosed herein (see, for example, Harlow and Lane, Antibodies: A Laboratory Manual, 2^{nd}, Cold Spring Harbor Laboratory, New York, 2013, Simpson ed., Basic methods in Protein Purification and Analysis: A laboratory Manual, Cold Harbor Press, 2008, and Ward et al., Nature 341:544, 1989.

In addition to recombinant methods, the antibodies, antigen binding fragments, and/or conjugates can also be constructed in whole or in part using standard peptide synthesis. Solid phase synthesis of the polypeptides can be accomplished by attaching the C-terminal amino acid of the sequence to an insoluble support followed by sequential addition of the remaining amino acids in the sequence. Techniques for solid phase synthesis are described by Barany & Merrifield, The Peptides: Analysis, Synthesis, Biology. Vol. 2: Special Methods in Peptide Synthesis, Part A. pp. 3-284; Merrifield et al., J. Am. Chem. Soc. 85:2149-2156, 1963, and Stewart et al., Solid Phase Peptide Synthesis, 2nd ed., Pierce Chem. Co., Rockford, Ill., 1984. Proteins of greater length may be synthesized by condensation of the amino and carboxyl termini of shorter fragments. Methods of forming peptide bonds by activation of a carboxyl terminal end (such as by the use of the coupling reagent N, N′-dicylohexylcarbodimide) are well known in the art.

### D. Methods and Compositions

1. Methods of Inhibiting, Treating, and Preventing EBOV Infection and Disease

Methods are disclosed herein for the prevention or treatment of an EBOV infection or EVD, such as a ZEBOV infection, in a subject. Prevention can include inhibition of infection with EBOV. The method can include administering to a subject a therapeutically effective amount of a disclosed antibody, antigen binding fragment, or conjugate that specifically binds EBOV GP, or a nucleic acid encoding such an antibody, antigen binding fragment, conjugate. In some examples, the antibody, antigen binding fragment, conjugate, or nucleic acid molecule, can be used pre-exposure (for example, to prevent or inhibit EBOV infection). In some examples, the antibody, antigen binding fragment, conjugate, or nucleic acid molecule, can be used in post-exposure prophylaxis. In some examples, the antibody, antigen binding fragment, conjugate, or nucleic acid molecule, can be used to eliminate or reduce the viral load of EBOV in a subject infected with EBOV. For example, a therapeutically effective amount of an antibody, antigen binding fragment, conjugate, or nucleic acid molecule, can be administered to a subject with an EBOV infection. In some examples the antibody, antigen binding fragment, conjugate, or nucleic acid molecule is modified such that it is directly cytotoxic to infected cells (for example, by conjugation to a toxin), or uses natural defenses such as complement, antibody dependent cellular cytotoxicity (ADCC), or phagocytosis by macrophages, or can be modified to increase the natural defenses.

The EVD or EBOV infection in the subject does not need to be completely eliminated for the method to be effective. For example, the method can reduce or ameliorate EVD or EBOV infection by a desired amount, for example by at least 10%, at least 20%, at least 50%, at least 60%, at least 70%, at least 80%, at least 90%, at least 95%, at least 98%, or even at least 100% (elimination of detectable EBOV infection or EVD), as compared to EBOV infection or EVD in the absence of the treatment.

In one non-limiting example, the method reduces viral titer in a subject with an EBOV infection. For example, administration of a therapeutically effective amount of a disclosed EBOV GP-specific antibody or antigen binding fragment or conjugate can reduce viral titer by at least 10%, at least 20%, at least 50%, at least 60%, at least 70%, at least 80%, at least 90%, at least 95%, at least 98%, or even at least 100% (elimination of detectable EBOV) in the subject. Methods of determining the EBOV viral titer in the subject are known, and include, for example, obtaining a blood sample from the subject and assaying the sample for EBOV activity.

In several embodiments, administration of a therapeutically effective amount of a disclosed antibody, antigen binding fragment, conjugate, or nucleic acid molecule, results in a reduction in the establishment of EBOV infection and/or reducing subsequent EVD progression in a subject. A reduction in the establishment of EBOV infection and/or a reduction in subsequent EVD progression encompass any statistically significant reduction in EBOV activity.

In several embodiments, the subject can be selected for treatment, for example, a subject at risk of EBOV infection, or known to have an EBOV infection. In some embodiments, a subject can be selected that is at risk of or known to have an infection with a particular strain of EBOV, such as BDBV, RESTV, SUDV, TAFV, or ZEBOV.

In several embodiments, a method of preventing or inhibiting EBOV infection (for example, ZEBOV infection) of a cell is provided. The method includes contacting the cell with an effective amount of an antibody or antigen binding fragment as disclosed herein. For example, the cell can be incubated with the effective amount of the antibody or antigen binding fragment prior to or contemporaneous with incubation with the EBOV. EBOV infection of the cell does not need to be completely eliminated for the method to be effective. For example, a method can reduce EBOV infection by a desired amount, for example by at least 10%, at least 20%, at least 50%, at least 60%, at least 70%, at least 80%, at least 90%, at least 95%, at least 98%, or even at least 100% (elimination of detectable EBOV infected cells), as compared to EBOV infection in the absence of the treatment. In some embodiments, the cell is also contacted with an effective amount of an additional agent, such as anti-viral agent. The cell can be in vivo or in vitro.

Studies in have shown that cocktails of EBOV neutralizing antibodies that target different epitopes of EBOV GP can treat macaques infected with ZEBOV (Qiu et al., Sci. Transl. Med., 4, 138ra81, 2012). Accordingly, in some examples, a subject is further administered one or more additional antibodies that bind EBOV GP and that can neutralize EBOV infection. For example, the subject can be administered a therapeutically effective amount of a set of antibodies including two or more of the EboV.YD.01, EboV.YD.02, EboV.YD.03, EboV.YD.04, EboV.YD.05, EboV.YD.06, EboV.YD.07 and EboV.YD.08 antibodies disclosed herein. The antibodies can be administered as a cocktail (that is, as a single composition including the two or more antibodies), or can be administered in sequence.

In some examples, a subject is administered the DNA encoding the antibody or antigen binding fragments thereof, to provide in vivo antibody production, for example using the cellular machinery of the subject. Immunization by nucleic acid constructs is well known in the art and taught, for example, in U.S. Pat. Nos. 5,643,578, and 5,593,972 and 5,817,637. U.S. Pat. No. 5,880,103 describes several methods of delivery of nucleic acids encoding to an organism. One approach to administration of nucleic acids is direct administration with plasmid DNA, such as with a mammalian expression plasmid. The nucleotide sequence encoding the disclosed antibody, or antigen binding fragments thereof, can be placed under the control of a promoter to increase expression. The methods include liposomal delivery of the nucleic acids. Such methods can be applied to the production of an antibody, or antigen binding fragments thereof. In some embodiments, a disclosed antibody or antigen binding fragment is expressed in a subject using the pVRC8400 vector (described in Barouch et al., J. Virol, 79:8828-8834, 2005, which is incorporated by reference herein).

The nucleic acid molecules encoding the disclosed antibodies or antigen binding fragments can be included in a viral vector, for example for expression of the antibody or antigen binding fragment in a host cell, or a subject (such as a subject with or at risk of EBOV infection). A number of viral vectors have been constructed, that can be used to express the disclosed antibodies or antigen binding fragments, such as a retroviral vector, an adenoviral vector, or an adeno-associated virus (AAV) vector. In several examples, the viral vector can be replication-competent. For example, the viral vector can have a mutation in the viral genome that does not inhibit viral replication in host cells. The viral vector also can be conditionally replication-competent. In other examples, the viral vector is replication-deficient in host cells.

In several embodiments, a subject (such as a human subject with or at risk of HIV-1 infection) can be administered a therapeutically effective amount of an adeno-associated virus (AAV) viral vector that includes one or more nucleic acid molecules encoding a disclosed antibody or antigen binding fragment. The AAV viral vector is designed for expression of the nucleic acid molecules encoding a disclosed antibody or antigen binding fragment, and administration of the therapeutically effective amount of the AAV viral vector to the subject leads to expression of a therapeutically effective amount of the antibody or antigen binding fragment in the subject. Non-limiting examples of AAV viral vectors that can be used to express a disclosed antibody or antigen binding fragment in a subject include those provided in Johnson et al (“Vector-mediated gene transfer engenders long-lived neutralizing activity and protection against SIV infection in monkeys,” Nat. Med., 15(8):901-906, 2009) and Gardner et al. (“AAV-expressed eCD4-Ig provides durable protection from multiple SHIV challenges,” Nature, 519(7541): 87-91, 2015), each of which is incorporated by reference herein in its entirety.

In one embodiment, a nucleic acid encoding a disclosed antibody, or antigen binding fragments thereof, is introduced directly into cells. For example, the nucleic acid can be loaded onto gold microspheres by standard methods and introduced into the skin by a device such as Bio-Rad's HELIOS™ Gene Gun. The nucleic acids can be “naked,” consisting of plasmids under control of a strong promoter.

Typically, the DNA is injected into muscle, although it can also be injected directly into other sites. Dosages for injection are usually around 0.5 μg/kg to about 50 mg/kg, and typically are about 0.005 mg/kg to about 5 mg/kg (see, e.g., U.S. Pat. No. 5,589,466).

2. Dosages

A therapeutically effective amount of an EBOV GP-specific antibody, antigen binding fragment, conjugate, or nucleic acid molecule encoding such molecules, will depend upon the severity of the disease and/or infection and the general state of the patient's health. A therapeutically effective amount is that which provides either subjective relief of a symptom(s) or an objectively identifiable improvement as noted by the clinician or other qualified observer. The EBOV GP-specific antibody, antigen binding fragment, conjugate, or nucleic acid molecule encoding such molecules, can be administered in conjunction with another therapeutic agent, either simultaneously or sequentially.

Single or multiple administrations of a composition including a disclosed EBOV GP-specific antibody, antigen binding fragment, conjugate, or nucleic acid molecule encoding such molecules, can be administered depending on the dosage and frequency as required and tolerated by the patient. Compositions including the EBOV GP-specific antibody, antigen binding fragment, conjugate, or nucleic acid molecule encoding such molecules, should provide a sufficient quantity of at least one of the EBOV GP-specific antibodies, antigen binding fragments, conjugates, or nucleic acid molecules to effectively treat the patient. The dosage can be administered once, but may be applied periodically until either a therapeutic result is achieved or until side effects warrant discontinuation of therapy. In one example, a dose of the antibody or antigen binding fragment is infused for thirty minutes every other day. In this example, about one to about ten doses can be administered, such as three or six doses can be administered every other day. In a further example, a continuous infusion is administered for about five to about ten days. The subject can be treated at regular intervals, such as daily, weekly, or monthly, until a desired therapeutic result is achieved. Generally, the dose is sufficient to treat or ameliorate symptoms or signs of disease without producing unacceptable toxicity to the patient.

Data obtained from cell culture assays and animal studies can be used to formulate a range of dosage for use in humans. The dosage normally lies within a range of circulating concentrations that include the ED₅₀, with little or minimal toxicity. The dosage can vary within this range depending upon the dosage form employed and the route of administration utilized. The therapeutically effective dose can be determined from cell culture assays and animal studies.

In certain embodiments, the antibody or antigen binding fragment that specifically binds EBOV GP, or conjugate thereof, or a nucleic acid molecule or vector encoding such a molecule, can be administered at a dose in the range of from about 1 to about 100 mg/kg, such as about 5-50 mg/kg, about 25-75 mg/kg, or about 40-60 mg/kg. In some embodiments, the dosage can be administered at about 1, 2, 3, 4, 5, 6, 7, 8, 9, 10, 20, 30, 40, 50, 60, 70, 80, 90, 100, 150, 200, or 300 mg/kg, or other dose deemed appropriate by the treating physician. Further, the doses described herein can be administered according to the dosing frequency or frequency of administration described herein, including without limitation daily, every other day, 2 or 3 times per week, weekly, every 2 weeks, every 3 weeks, monthly, etc. In some embodiments, the dosage is administered daily beginning at the time of diagnosis with EBOV and until EBOV symptoms are alleviated. Additional treatments, including additional courses of therapy with a disclosed agent can be performed as needed.

3. Modes of Administration

The EBOV GP-specific antibody, antigen binding fragment, conjugate, nucleic acid molecule, or composition, as well as additional agents, can be administered to subjects in various ways, including local and systemic administration, such as, for example, by injection subcutaneously, intravenously, intra-arterially, intraperitoneally, intramuscularly, intradermally, or intrathecally. In an embodiment, a therapeutic agent is administered by a single subcutaneous, intravenous, intra-arterial, intraperitoneal, intramuscular, intradermal or intrathecal injection once a day. The therapeutic agent can also be administered by direct injection at or near the site of disease.

The therapeutic agent may also be administered orally in the form of microspheres, microcapsules, liposomes (uncharged or charged (such as cationic)), polymeric microparticles (such as polyamides, polylactide, polyglycolide, poly(lactide-glycolide)), microemulsions, and the like.

A further method of administration is by osmotic pump (for example, an Alzet pump) or mini-pump (for example, an Alzet mini-osmotic pump), which allows for controlled, continuous and/or slow-release delivery of the therapeutic agent or pharmaceutical composition over a pre-determined period. The osmotic pump or mini-pump can be implanted subcutaneously, or near a target site.

It will be apparent to one skilled in the art that the therapeutic agent or compositions thereof can also be administered by other modes. The therapeutic agent can be administered as pharmaceutical formulations suitable for, for example, oral (including buccal and sub-lingual), rectal, nasal, topical, pulmonary, vaginal or parenteral (including intramuscular, intraarterial, intrathecal, subcutaneous and intravenous) administration, or in a form suitable for administration by inhalation or insufflation. Depending on the intended mode of administration, the pharmaceutical formulations can be in the form of solid, semi-solid or liquid dosage forms, such as tablets, suppositories, pills, capsules, powders, liquids, suspensions, emulsions, creams, ointments, lotions, and the like. The formulations can be provided in unit dosage form suitable for single administration of a precise dosage. The formulations comprise an effective amount of a therapeutic agent, and one or more pharmaceutically acceptable excipients, carriers and/or diluents, and optionally one or more other biologically active agents.

4. Compositions

Compositions are provided that include one or more of the disclosed EBOV GP-specific antibodies, antigen binding fragments, conjugates, or nucleic acid molecules, in a carrier. The compositions are useful, for example, for the treatment or detection of an EBOV infection. The compositions can be prepared in unit dosage forms for administration to a subject. The amount and timing of administration are at the discretion of the treating physician to achieve the desired purposes. The EBOV GP-specific antibody, antigen binding fragment, conjugate, or nucleic acid molecule encoding such molecules can be formulated for systemic or local administration. In one example, the EBOV GP-specific antibody, antigen binding fragment, conjugate, or nucleic acid molecule encoding such molecules, is formulated for parenteral administration, such as intravenous administration.

In some embodiments, the compositions comprise an antibody, antigen binding fragment, or conjugate thereof, in at least 70%, at least 75%, at least 80%, at least 85%, at least 90%, at least 95%, at least 96%, at least 97%, at least 98% or at least 99% purity. In certain embodiments, the compositions contain less than 10%, less than 5%, less than 4%, less than 3%, less than 2%, less than 1% or less than 0.5% of macromolecular contaminants, such as other mammalian (for example, human) proteins.

The compositions for administration can include a solution of the EBOV GP-specific antibody, antigen binding fragment, conjugate, or nucleic acid molecule encoding such molecules, dissolved in a pharmaceutically acceptable carrier, such as an aqueous carrier. A variety of aqueous carriers can be used, for example, buffered saline and the like. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of antibody in these formulations can vary widely, and will be selected primarily based on fluid volumes, viscosities, body weight and the like in accordance with the particular mode of administration selected and the subject's needs.

A typical composition for intravenous administration includes about 0.01 to about 30 mg/kg of antibody or antigen binding fragment or conjugate per subject per day (or the corresponding dose of a conjugate including the antibody or antigen binding fragment). Actual methods for preparing administrable compositions will be known or apparent to those skilled in the art and are described in more detail in such publications as Remington's Pharmaceutical Science, 22th ed., Pharmaceutical Press, London, UK (2012). In some embodiments, the composition can be a liquid formulation including one or more antibodies, antigen binding fragments (such as an antibody or antigen binding fragment that specifically binds to EBOV GP), in a concentration range from about 0.1 mg/ml to about 20 mg/ml, or from about 0.5 mg/ml to about 20 mg/ml, or from about 1 mg/ml to about 20 mg/ml, or from about 0.1 mg/ml to about 10 mg/ml, or from about 0.5 mg/ml to about 10 mg/ml, or from about 1 mg/ml to about 10 mg/ml.

The disclosed antibodies, antigen binding fragments, conjugates, and nucleic acid encoding such molecules, can be provided in lyophilized form and rehydrated with sterile water before administration, although they are also provided in sterile solutions of known concentration. The antibody solution, or an antigen binding fragment or a nucleic acid encoding such antibodies or antigen binding fragments, can then be added to an infusion bag containing 0.9% sodium chloride, USP, and administered according to standard protocols. Considerable experience is available in the art in the administration of antibody drugs, which have been marketed in the U.S. since the approval of RITUXAN® in 1997. Antibodies, antigen binding fragments, conjugates, or a nucleic acid encoding such molecules, can be administered by slow infusion, rather than in an intravenous push or bolus. In one example, a higher loading dose is administered, with subsequent, maintenance doses being administered at a lower level. For example, an initial loading dose of 4 mg/kg may be infused over a period of some 90 minutes, followed by weekly maintenance doses for 4-8 weeks of 2 mg/kg infused over a 30 minute period if the previous dose was well tolerated.

Controlled-release parenteral formulations can be made as implants, oily injections, or as particulate systems. For a broad overview of protein delivery systems see, Banga, A. J., Therapeutic Peptides and Proteins: Formulation, Processing, and Delivery Systems, Technomic Publishing Company, Inc., Lancaster, Pa., (1995). Particulate systems include microspheres, microparticles, microcapsules, nanocapsules, nanospheres, and nanoparticles. Microcapsules contain the therapeutic protein, such as a cytotoxin or a drug, as a central core. In microspheres the therapeutic is dispersed throughout the particle. Particles, microspheres, and microcapsules smaller than about 1 μm are generally referred to as nanoparticles, nanospheres, and nanocapsules, respectively. Capillaries have a diameter of approximately 5 μm so that only nanoparticles are administered intravenously. Microparticles are typically around 100 μm in diameter and are administered subcutaneously or intramuscularly (see, for example, Kreuter, Colloidal Drug Delivery Systems, J. Kreuter, ed., Marcel Dekker, Inc., New York, N.Y., pp. 219-342 (1994); and Tice & Tabibi, Treatise on Controlled Drug Delivery, A. Kydonieus, ed., Marcel Dekker, Inc. New York, N.Y., pp. 315-339, (1992).

Polymers can be used for ion-controlled release of the antibody compositions disclosed herein. Various degradable and nondegradable polymeric matrices for use in controlled drug delivery are known in the art (Langer, Accounts Chem. Res. 26:537-542, 1993). For example, the block copolymer, polaxamer 407, exists as a viscous yet mobile liquid at low temperatures but forms a semisolid gel at body temperature. It has been shown to be an effective vehicle for formulation and sustained delivery of recombinant interleukin-2 and urease (Johnston et al., Pharm. Res. 9:425-434, 1992; and Pec et al., J. Parent. Sci. Tech. 44(2):58-65, 1990). Alternatively, hydroxyapatite has been used as a microcarrier for controlled release of proteins (Ijntema et al., Int. J. Pharm. 112:215-224, 1994). In yet another aspect, liposomes are used for controlled release as well as drug targeting of the lipid-capsulated drug (Betageri et al., Liposome Drug Delivery Systems, Technomic Publishing Co., Inc., Lancaster, Pa. (1993)). Numerous additional systems for controlled delivery of therapeutic proteins are known (see U.S. Pat. Nos. 5,055,303; 5,188,837; 4,235,871; 4,501,728; 4,837,028; 4,957,735; 5,019,369; 5,055,303; 5,514,670; 5,413,797; 5,268,164; 5,004,697; 4,902,505; 5,506,206; 5,271,961; 5,254,342 and 5,534,496).

5. Methods of Detection and Diagnosis

Methods are also provided for the detection of the expression of EBOV GP in vitro or in vivo. In one example, expression of EBOV GP is detected in a biological sample, and can be used to detect EBOV infection as the presence of EBOV in a sample. The sample can be any sample, including, but not limited to, tissue from biopsies, autopsies and pathology specimens. Biological samples also include sections of tissues, for example, frozen sections taken for histological purposes. Biological samples further include body fluids, such as blood, serum, plasma, sputum, spinal fluid or urine. The method of detection can include contacting a cell or sample, or administering to a subject, an antibody or antigen binding fragment that specifically binds to EBOV GP, or conjugate there of (such as a conjugate including a detectable marker) under conditions sufficient to form an immune complex, and detecting the immune complex (for example, by detecting a detectable marker conjugated to the antibody or antigen binding fragment.

In several embodiments, a method is provided for detecting EBOV disease and/or an EBOV infection in a subject. The disclosure provides a method for detecting EBOV in a biological sample, wherein the method includes contacting a biological sample from a subject with a disclosed antibody or antigen binding fragment under conditions sufficient for formation of an immune complex, and detecting the immune complex, to detect the EBOV GP in the biological sample. In one example, the detection of EBOV GP in the sample indicates that the subject has an EBOV infection. In another example, the detection of EBOV GP in the sample indicates that the subject has EVD. In another example, detection of EBOV GP in the sample confirms a diagnosis of EVD and/or an EBOV infection in the subject.

In some embodiments, the disclosed antibodies or antigen binding fragments are used to test vaccines. For example, to test if a vaccine composition including EBOV GP assumes a conformation including the EBOV GP epitope to which EboV.YD.01, EboV.YD.02, EboV.YD.03, EboV.YD.04, EboV.YD.05, EboV.YD.06, EboV.YD.07, or EboV.YD.08 antibody binds. Thus provided herein is a method for testing a vaccine, wherein the method includes contacting a sample containing the vaccine, such as an EBOV GP immunogen, with a disclosed antibody or antigen binding fragment under conditions sufficient for formation of an immune complex, and detecting the immune complex. Detection of the immune complex confirms that the EBOV GP vaccine includes the epitope to which EboV.YD.01, EboV.YD.02, EboV.YD.03, EboV.YD.04, EboV.YD.05, EboV.YD.06, EboV.YD.07, or EboV.YD.08 antibody, respectively binds. In one example, the detection of the immune complex in the sample indicates that a vaccine component, such as an EBOV GP immunogen assumes a conformation capable of binding the antibody or antigen binding fragment.

In one embodiment, the antibody or antigen binding fragment is directly labeled with a detectable marker. In another embodiment, the antibody that binds EBOV GP (the first antibody) is unlabeled and a second antibody or other molecule that can bind the antibody that binds the first antibody is utilized for detection. As is well known to one of skill in the art, a second antibody is chosen that is able to specifically bind the specific species and class of the first antibody. For example, if the first antibody is a human IgG, then the secondary antibody may be an anti-human-IgG. Other molecules that can bind to antibodies include, without limitation, Protein A and Protein G, both of which are available commercially.

Suitable labels for the antibody, antigen binding fragment or secondary antibody are described above, and include various enzymes, prosthetic groups, fluorescent materials, luminescent materials, magnetic agents and radioactive materials. Non-limiting examples of suitable enzymes include horseradish peroxidase, alkaline phosphatase, beta-galactosidase, or acetylcholinesterase. Non-limiting examples of suitable prosthetic group complexes include streptavidin/biotin and avidin/biotin. Non-limiting examples of suitable fluorescent materials include umbelliferone, fluorescein, fluorescein isothiocyanate, rhodamine, dichlorotriazinylamine fluorescein, dansyl chloride or phycoerythrin. A non-limiting exemplary luminescent material is luminol; a non-limiting exemplary a magnetic agent is gadolinium, and non-limiting exemplary radioactive labels include ¹²⁵I, ¹³¹I, ³⁵S or ³H.

### E. Kits

Kits are also provided. For example, kits for treating a subject with an EBOV infection, or for detecting EBOV GP in a sample or in a subject. The kits will typically include a disclosed EBOV GP-specific antibody, antigen binding fragment, or nucleic acid molecule encoding such molecules, or compositions including such molecules. More than one of the disclosed EBOV GP-specific antibody, antigen binding fragment, conjugate, or nucleic acid molecule encoding such molecules, or compositions including such molecules can be included in the kit.

In one embodiment, the kit is a diagnostic kit and includes an immunoassay. Although the details of the immunoassays may vary with the particular format employed, the method of detecting EBOV GP in a biological sample generally includes the steps of contacting the biological sample with an antibody which specifically reacts, under conditions sufficient to form an immune complex, to EBOV GP. The antibody is allowed to specifically bind under immunologically reactive conditions to form an immune complex, and the presence of the immune complex (bound antibody) is detected directly or indirectly.

The kit can include a container and a label or package insert on or associated with the container. Suitable containers include, for example, bottles, vials, syringes, etc. The containers may be formed from a variety of materials such as glass or plastic. The container typically holds a composition including one or more of the disclosed antibodies, antigen binding fragments, conjugates, nucleic acid molecules, or compositions. In several embodiments the container may have a sterile access port (for example the container may be an intravenous solution bag or a vial having a stopper pierceable by a hypodermic injection needle). A label or package insert indicates that the composition is used for treating the particular condition.

The label or package insert typically will further include instructions for use of the antibodies, antigen binding fragments, conjugates, nucleic acid molecules, or compositions included in the kit. The package insert typically includes instructions customarily included in commercial packages of therapeutic products that contain information about the indications, usage, dosage, administration, contraindications and/or warnings concerning the use of such therapeutic products. The instructional materials may be written, in an electronic form or may be visual. The kits may also include additional components to facilitate the particular application for which the kit is designed. Thus, for example, the kit may additionally contain means of detecting a label (such as enzyme substrates for enzymatic labels, filter sets to detect fluorescent labels, appropriate secondary labels such as a secondary antibody, or the like). The kits may additionally include buffers and other reagents routinely used for the practice of a particular method. Such kits and appropriate contents are well known to those of skill in the art.

The following examples are provided to illustrate certain particular features and/or embodiments. These examples should not be construed to limit the disclosure to the particular features or embodiments described.

### Examples

### Example 1: Materials and Methods

This example describes the materials and experimental procedures for the studies described in Example 2.

### Strain and Media

The yeast strain AWY101 (MATα AGA1::GAL1-AGA1::URA3 PD11::GAPDH-PDI1::LEU2 ura3-52 trp1 leu2Δ1 his3Δ200 pep4::HIS3 prb1Δ1.6R can1 GAL) was used for library construction and screening. EBY100 (MATα AGA1::GAL1-AGA1::URA3 ura3-52 trp1 leu2Δ1 his3Δ200 pep4::HIS3 prb1Δ1.6R can1 GAL) was used for initial native human antibody display. Yeast cells were maintained in YPD medium (20 g/l dextrose, 20 g/l peptone, and 10 g/l yeast extract); after library transformation, yeast cells were maintained in SDCAA medium (20 g/l dextrose, 6.7 g/l yeast nitrogen base, 5 g/l casamino acids, 8.56 g/l NaH₂PO₄.H₂O, and 10.2 g/l Na₂HPO₄.7H₂O). SGDCAA medium (SDCAA with 20 g/l galactose, 2 g/l dextrose) was used for library induction.

### Antigens and Antibodies

Recombinant Ebola virus glycoprotein with the mucin-like domain deleted (GP_{Δmuc}), and HIV-1 fusion peptide probe (VRC34-epitope scaffold-FP) and knockout scaffold probe (VRC34-epitope scaffold-KO) were produced as described previously (Kong et al., Science 352, 828-833, 2016; Côté et al., Nature 477, 344-348, 2011; Misasi et al., Science 351, 1343-1346, 2016). Proteins were biotinylated and conjugated with streptavidin-APC (GP_{Δmuc} and VRC34-epitope scaffold-FP) or streptavidin-PE (VRC34-epitope scaffold-KO) (Thermo Fisher Scientific), respectively. Recombinant hemagglutinins (A/California/07/2009, A/Solomon Islands/3/2006, and A/Wisconsin/67/2005) were produced as before (Whittle et al. Proc. Natl. Acad. Sci. USA 108, 14216-14221, 2011) or acquired from BEI Resources (A/Victoria/210/2009, and B/Brisbane/60/2008) and were biotinylated using an EZ-Link Sulfo-NHS-LCBiotin kit (Thermo Fisher Scientific). Anti-FLAG fluorescein isothiocyanate (FITC) antibody was purchased from Sigma-Aldrich (clone M2).

### Optimization of Native Human Antibody Display

Vectors encoding previously reported anti-influenza virus HA monoclonal antibodies with or without leucine zipper domains were transformed into EBY100 or AWY101 using a Frozen-EZ Yeast Transformation II kit (Zymo Research) (Lee et al., Nat. Med. 22, 1456-1464, 2016). After culturing in SDCAA to an OD₆₀₀ of 2 at 30° C., Fab surface expression was induced by transferring cells to SGDCAA medium at OD₆₀₀ of 0.5. After 2 days of induction at 20° C., 10⁶ cells were collected and washed twice with PBS+0.5% BSA+2 mM EDTA, and incubated with 100 nM biotinylated hemagglutinin at room temperature for 30 minutes, followed by staining with 2 μg/ml anti-FLAG FITC and 2 μg/ml streptavidin-APC at 4° C. for 15 minutes. Cells were washed twice with ice-cold PBS+0.5% BSA+2 mM EDTA and analyzed on a FACS Aria II (BD Biosciences). Analysis of anti-EBOV GP_{Δmuc} (c13c6, KZ52) and anti-HIV-1 FP (VRC34.01) antibodies was performed as above (Kong et al., Science 352, 828-833, 2016; Lee et al., Nature 454, 177-182, 2008; Olinger et al., Proc. Natl. Acad. Sci. USA 109, 18030-18035, 2012), except 23 nM GP_{Δmuc}-APC or 50 nM VRC34-epitope scaffold-FP-APC and 50 nM VRC34-epitope scaffold-KO-PE, respectively, were used for antigen staining.

Generation of Natively Paired VH:VL from Peripheral B Cells, Library Construction, Yeast Display and FACS Screening

For anti-EBOV GP_{Δmuc} antibody isolation, peripheral blood mononuclear cells (PBMCs) were isolated from a healthy human volunteer after immunization with a phase 1 Ebola GP vaccine (NCT02408913) (Stanley et al., Nat. Med. 20, 1126-1129, 2014). The volunteer was first immunized with chimpanzee-derived replication-defective adenovirus encoding EBOV GP, then boosted 30 weeks and 5 days later with modified vaccinia Ankara encoding EBOV GP. Previous studies showed that plasmablasts in peripheral blood peak around 6 days post-boost immunization (Lavinder et al., Proc. Natl. Acad. Sci. USA 111, 2259-2264, 2014). Ten ml of blood was collected and PBMCs isolated using Ficoll-Paque PLUS (GE Healthcare) 6 days post-boost. PBMCs were stained with a multi-color flow cytometry panel consisting of fluorophore-labeled antibodies against CD3 (Brilliant Violet 421, clone SP34-2, BD Biosciences), CD19 (PE-Cγ7, clone HIB19, BD Biosciences), CD4 (Brilliant Violet 421, clone OKT4, BioLegend), CD8a (Brilliant Violet 421, clone RPAT8, BioLegend), CD14 (Brilliant Violet 421, clone M5E2, BioLegend), CD20 (Brilliant Violet 605, clone 2H7, BioLegend), CD27 (Brilliant Violet 711, clone O323, BioLegend), and CD38 (Alexa Fluor 680, clone OKT10, custom-conjugated at the Vaccine Research Center, NIAID), and 7-aminoactinomycin D (7-AAD, Thermo Fisher Scientific) to exclude dead cells. 5,002 CD3⁻CD4⁻CD8⁻CD14⁻CD19⁺CD20⁻CD27⁺CD38⁺ plasmablasts were isolated using a FACS Aria sorter (BD Biosciences) and subsequently used for emulsion VH:VL overlap extension RT-PCR.

For HIV-1 FP antibody isolation, PBMCs were collected from donor N123 on Jun. 22, 2009. This donor is a chronically HIV-1-infected individual (Doria-Rose et al., J. Virol. 83, 188-199, 2009; Doria-Rose et al., J. Virol. 84, 1631-1636, 2010). This donor was diagnosed with HIV-1 in 2000. After more than nine years of infection, this donor showed a CD4 T cell count of 463 cells/ml and a plasma HIV-1 viral load of 4,920 RNA copies/ml. This donor was not on antiretroviral treatment. 1.42×10⁶ peripheral B cells were isolated from 25 million PBMCs using a human B-cell selection kit (Stemcell Technologies).

For the isolation of influenza HA-specific antibodies, a healthy donor was vaccinated with a trivalent inactivated influenza vaccine (IIV3: A/Solomon Islands/3/2006, A/Wisconsin/67/2005, B/Malaysia/2508/2004)24. Subsequently, 270 days after vaccination, 1.2×10⁷B cells were isolated from blood leukapheresis using a human pan B-cell isolation kit (Miltenyi Biotec).

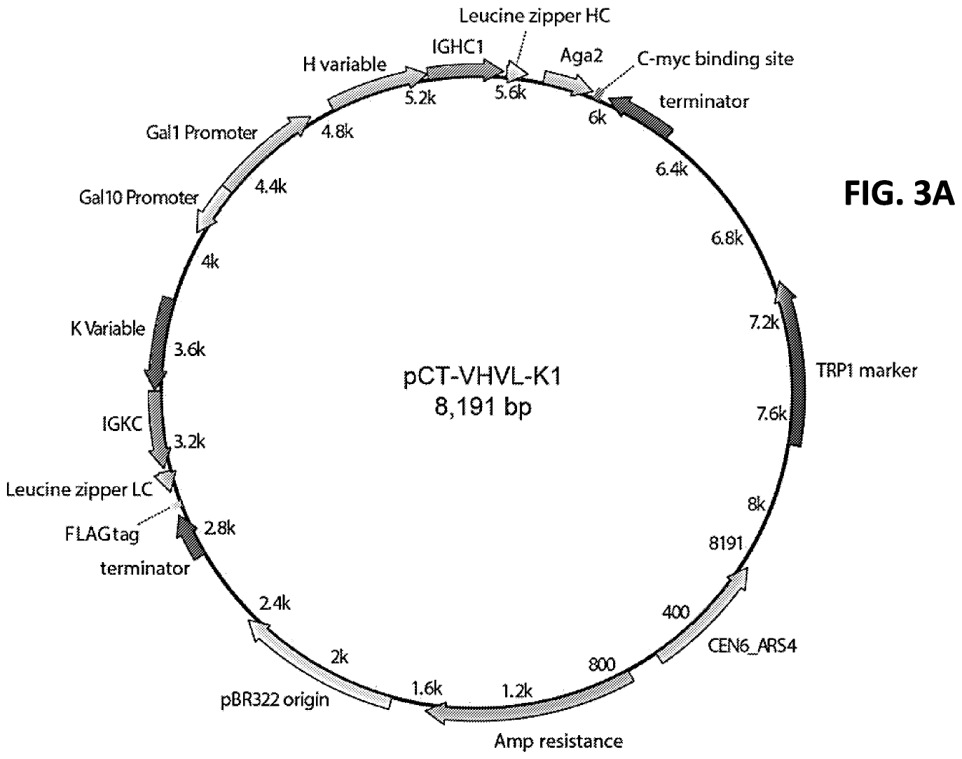

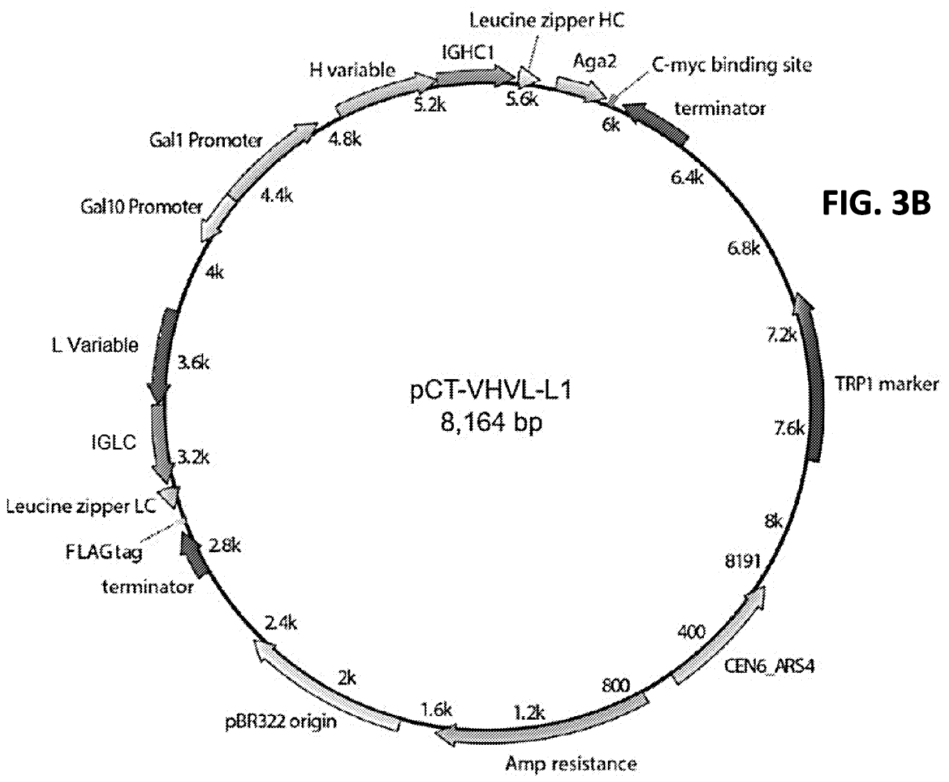

A flow-focusing nozzle was used to rapidly compartmentalize B cells in single-cell emulsion droplets, followed by single-B-cell lysis inside droplets and single-cell mRNA capture with oligo(dT)-coated magnetic beads (DeKosky et al., Nat. Med. 21, 86-91, 2015; McDaniel et al., Nat. Protoc. 11, 429-442, 2016). Overlap extension RT-PCR was then performed to link heavy and light chains using a Superscript III RT-PCR kit (Thermo Fisher Scientific) (DeKosky et al., Nat. Med. 21, 86-91, 2015; McDaniel et al., Nat. Protoc. 11, 429-442, 2016). NcoI and NheI restriction sites were included in the linker region of the overlap-extension RTPCR primers that link VH and VL into an ˜850 bp amplicon. For HIV-1 experiments, additional primers specific to the VRC34 lineage were also included. For library construction, 100 ng of VH:VL cDNA was amplified under the following conditions with AccuPrime Pfx DNA polymerase (Thermo Fisher Scientific) to introduce NotI and AscI sites, respectively (Table 1): 2 minute initial denaturation at 95° C., denaturation at 95° C. for 20 seconds for 20 cycles, annealing at 60° C. for 20 seconds and extension at 68° C. for 60 seconds, final extension at 68° C. for 5 minutes. The DNA product was digested and ligated into pCT-VHVL-K1 (for VH:Vκ libraries; FIG. 3A) and pCT-VHVL-L1 (for VH:Vλ libraries; FIG. 3B), and transformed into electrocompetent E. coli for library cloning en masse. Plasmid DNA encoding VH:VL libraries was miniprepped, digested with NcoI and NheI, ligated with the bidirectional promoter, and transformed into E. coli again. The final library DNA was miniprepped, then amplified using the transformation primers (Table 1) to generate library inserts with homologous ends to NotI and AscI double-digested vectors, and then inserts were co-transformed into electrocompetent AWY101 together with the digested vectors to generate libraries via yeast homologous recombination (Benatuil et al., Protein Eng. Des. Sel. 23, 155-159, 2010). Library sizes for EBOV, HIV-1 and flu repertoires were EBOV_κ: 2×10⁷, EBOV_λ: 10⁷, HIV-1_λ: 10⁷, HIV-1_κ: 9×10⁶, flu_κ: 7×10⁷, and flu_λ: 3×10⁷, respectively, as determined by colony counting.

TABLE 1
Yeast display cloning and transformation primers

| | | | |
|---|---|---|---|
| Conc. | | | |
| (nM) | Primer ID | Primer Sequence | SEQ ID NO: |

Cloning

| | | | |
|---|---|---|---|
| 200 | hVH1.rev | GTTCTAGGCGCGCCTGTACTTGCTGAGGAG | 34 |
| | | ACRGTGACCAGGGTG | |
| 200 | hVH3.rev | GTTCTAGGCGCGCCTGTACTTGCTGAAGAG | 35 |
| | | ACGGTGACCATTGT | |
| 200 | hVH4.rev | GTTCTAGGCGCGCCTGTACTTGCTGAGGAG | 36 |
| | | ACGGTGACCAGGGT | |
| 200 | hVH6.rev | GTTCTAGGCGCGCCTGTACTTGCTGAGGAG | 37 |
| | | ACGGTGACCGTGGTCC | |
| 200 | hVKl.rev | CTTATAGCGGCCGCAGTTCGTTTGATTTCC | 38 |
| | | ACCTTGGTCC | |
| 200 | hVK2.rev | CTTATAGCGGCCGCAGTTCGTTTGATCTCC | 39 |
| | | ASCTTGGTCC | |
| 200 | hVK3.rev | CTTATAGCGGCCGCAGTTCGTTTGATATCC | 40 |
| | | ACTTTGGTCC | |
| 200 | hVK5.rev | CTTATAGCGGCCGCAGTTCGTTTAATCTCC | 41 |
| | | AGTCGTGTCC | |
| 200 | hVL1.rev | CTTATAGCGGCCGCGGGCTGACCTAGGACG | 42 |
| | | GTSASCTTGGTCC | |
| 200 | hVL4.rev | CTTATAGCGGCCGCGGGCTGACCTAAAATG | 43 |
| | | ATCAGCTGGGTTC | |
| 200 | hVL5 .rev | CTTATAGCGGCCGCGGGCTGACCTAGGACG | 44 |
| | | GTCAGCTCSGTCC | |
| 200 | hVL6.rev | CTTATAGCGGCCGCGGGCTGACCGAGGACG | 45 |
| | | GTCACTTGGTCCA | |
| 200 | hVL7 .rev | CTTATAGCGGCCGCGGGCTGACCGAGGRCG | 46 |
| | | GTCAGCTGGGTGC | |

Yeast Transform.

| | | | |
|---|---|---|---|
| 200 | YD.hu.H.transform | GGAAGTAGTCCTTGACCAGGC | 47 |
| 200 | YD.hu.K.transform | CTCTCTGGGATAGAAGTTATTCAGC | 48 |
| 200 | YD.hu.L.transform | CCAGGGTAGCTTTGTTCGCTTGC | 49 |

For library screening, natively paired human VH:VL libraries were displayed on yeast by growing cells resuspended in SGDCAA medium at 20° C. for 2 days to induce Fab expression. The fraction of Fab-expressing cells 2 days post induction were consistent with previous reports for yeast displaying naïve human scFv15. For EBOV vaccinee and HIV-1 donor libraries, three rounds of sorting were performed against GP_{Δmuc}, or VRC34-epitope scaffold-FP-APC and VRC34-epitope scaffold-KO-PE, respectively. The VRC34-epitope scaffold-FP was designed to present the FP in an optimal conformation and provide a glycan in a similar context as that presented by the native HIV-1 trimer (Kong et al., Science 352, 828-833, 2016). In the first round of screening, at least tenfold coverage in yeast clones relative to library size were labeled with 2 μg/ml anti-FLAG-FITC and either (i) 23 nM GP_{Δmuc}-APC for isolating of EBOV GP_{Δmuc}-specific antibodies, or (ii) 50 nM VRC34-epitope scaffold-FP-APC and 50 nM VRC34-epitope scaffold-KO-PE for the isolation of HIV-1 FP-specific antibodies. For EBOV antibody libraries, the PE channel was also included to correct for yeast autofluorescence. Cells were stained at room temperature for 30 minutes and washed twice with ice-cold PBS+0.5% BSA+2 mM EDTA, then analyzed by FACS. FITC⁺APC⁺PE⁻ cells were selected and recovered in SDCAA medium at 30° C. Subsequent screening rounds were performed similarly, except that for the EBOV GP antibody library, at least 5×10⁵ cells were screened in rounds 2 and 3, and for HIV-1 FP antibody library, at least 10⁷ cells were screened in rounds 2 and 3. Affinity binning of anti-EBOV GP_{Δmuc} and anti-HIV-1 FP antibody repertoires was performed similarly as described (Reich et al., J. Mol. Biol. 427, 2135-2150, 2015).

Influenza HA-specific antibodies were isolated following five total rounds of sorting (two rounds of MACS (magnetic-activated cell sorting) and three rounds of FACS enrichment for binding to fluorescent HAs) as follows. For the first round, at least tenfold coverage in yeast clones relative to library size were labeled with 2 μg/ml anti-FLAG-FITC at room temperature for 30 minutes. After washing, cells were labeled with anti-FITC microbeads (Miltenyi Biotec) at 4° C. for 15 minutes, and Fab-expressing cells were selected by MACS. For the second round of sorting, cells were labeled with 1 μM biotinylated recombinant HA (H1 A/Solomon Islands/3/2006, H3 A/Wisconsin/67/2005), and then selected with streptavidin microbeads (Miltenyi Biotec) using MACS as previously described (Wang et al., MAbs 8, 1035-1044, 2016). Subsequently the library was screened using three rounds of FACS by labeling 5×10⁶ cells with 2 μg/ml anti-FLAG-FITC together with 1 μM HA (first FACS round), 200 nM HA (second FACS round), or 40 nM HA (third FACS round), at room temperature for 30 minutes, followed by incubation with 2 μg/ml streptavidin-APC at 4° C. for 15 minutes before sorting.

Sequencing of the natively paired antibody repertoire from B cells and bioinformatic analysis were performed as previously described (DeKosky et al., Proc. Natl. Acad. Sci. USA 113, E2636-E2645, 2016).

Recovery and Expression of Antibody Clones from Enriched Libraries

Yeast cells from the final round of sorting were plated on SDCAA plates. A minimum of ten colonies were selected following the last round of each screening campaign. Colony PCR was performed on heavy and light chain variable regions of each clone. Clones were sequenced, and the unique antibodies were named as project_name.YD.unique_clone_number. For antibody expression, restriction sites were incorporated for insertion into the VRC8400 IgG1, and Igκ or Igλ, expression vectors (for anti-EBOV GP_{Δmuc} and HIV-1 FP antibodies), or Gibson assembly was used to clone the variable regions into modified pcDNA3.4 IgG1, and Igκ or Igλ vectors (for anti-HA antibodies). Expi293 cells were co-transfected with heavy- and light-chain plasmids for each antibody, and secreted antibodies were purified on a Protein A column (Cale et al., Immunity 46, 777-791.e10, 2017). Fabs were produced by digestion of IgG1 with Lys-C Protease (Thermo Fisher Scientific) and separated from Fc using Protein A or Protein G columns.

For EBOV vaccinee libraries, the population of sorted FITC Fab-expressing yeast cells in the first round of FACS were recovered in SDCAA medium at 30° C. Plasmid DNA was extracted using high-efficiency yeast plasmid recovery protocols as reported previously (Whitehead et al., Nat. Biotechnol. 30, 543-548, 2012) and VH genes in sorted libraries were PCR-amplified using primers that targeted the yeast expression plasmid vector backbone:

2YDrec_heavy_Vfor_MSrev1
(SEQ ID NO: 50)
TCTCGTGGGCTCGGAGATGTGTATAAGAGACAGNNNNCTGTTATTGCTAG
CGTTTTAGCA
2YDrec_huIgH_Crev_MSfor1
(SEQ ID NO: 51)
TCGTCGGCAGCGTCAGATGTGTATAAGAGACAGNNNNAAGGCGCGCCTGT
ACTTGC

Libraries were prepped by an additional round of PCR-based primer extension and Illumina adaptor addition to incorporate unique DNA barcodes for each sample, and sequenced using the Illumina 2×300 bp MiSeq platform. Similarly, FACS-sorted yeast from EBOV vaccinee libraries analyzed for binding to GP_{Δmuc} and FACS-sorted yeast from HIV-1 donor libraries analyzed for binding to VRC34-epitope scaffold-FP-APC were recovered, mini-prepped, and sequenced in the same way after each round of sorting to quantify VH gene clonal enrichment across library sorting rounds.

For determining the EBOV vaccinee antibody library display efficiency, the original plasmablast VH:VL repertoire underwent highly stringent quality filtering (≥15 CDRH3:CDRL3 reads, 96% CDRH3 nt clustering), and CDR-H3 nucleotide junctions were mapped to CDR-H3 junctions recovered in the FITC Fab-expressing yeast library. Mapping was performed using usearch v5.2.32 with an exact nucleotide length match requirement and a 96% cutoff threshold for CDR-H3 junction nucleotide sequence match. For EBOV GP_{Δmuc} library antibody discovery via NGS (next-generation sequencing) clonal lineage tracking, CDR-H3 amino acid sequences from NGS data sets that were enriched more than eightfold across multiple rounds of screening were synthesized, expressed in HEK293 cells, and tested for soluble binding to GP_{Δmuc} as Fabs (EBOV.YD.05-EBOV.YD.08). Consensus sequences for NGS-discovered antibodies were generated based on exact CDR-H3 and CDR-L3 nucleotide junction matches between the originally paired VH:VL, separate VH, and separate VL sequencing libraries (before yeast display screening), as previously described for antibody discovery from paired heavy:light sequence data sets (DeKosky et al., Proc. Natl. Acad. Sci. USA 113, E2636-E2645, 2016; DeKosky et al., Nat. Biotechnol. 31, 166-169, 2013; Wang et al., Sci. Rep. 5, 13926, 2015). Briefly, consensus sequences were generated using usearch v5.2.32 from exact match reads to the CDR-H3 nucleotide or CDR-L3 nucleotide junctions for heavy or light chains, respectively, and plasmids containing antibody heavy and light chain sequences were expressed via transient transfection in HEK293 cells for soluble antibody generation as previously described (Misasi et al., Science 351, 1343-1346, 2016).

### Affinity Characterization

Binding kinetics of anti-EBOV GP_{Δmuc} and anti-HIV-1 FP Fabs were determined using biolayer interferometry on a FortéBio Octet HTX instrument (Misasi et al., Science 351, 1343-1346, 2016). For EBOV GP_{Δmuc}-targeting antibodies, AR2G biosensors were coupled with GP_{Δmuc} (10 μg/ml in 10 mM acetate, pH 4.5) for 600 seconds. Typical capture levels after quenching with 1 M ethanolamine (pH 8.0) for 300 seconds were between 2 and 2.5 nm, and variability within the same protein did not exceed 0.25 nm. Biosensors were then equilibrated for 420 seconds in PBST-BSA (PBS+1% BSA+0.01% Tween+0.02% sodium azide) before binding assessment of the Fab. Association of Fab was measured for 300-600 seconds and dissociation was measured for 300-600 seconds, both in PBST-BSA. Correction to subtract nonspecific baseline drift was carried out by subtracting the measurements recorded for a sensor loaded with unrelated antigen (HIV-1 gp120).

For HIV-1 FP-targeting antibodies, streptavidin biosensors were used to capture VRC34-epitope scaffold-FP at 0.5 μg/mL in PBST-BSA. Typical capture levels for FP probe were between 0.4 and 0.7 nm. Biosensors were then equilibrated for 60 seconds in PBST-BSA before binding assessment of the Fab. Association of Fab was measured for 150 seconds and dissociation was measured for 150 seconds, both in PBST-BSA. Correction to subtract non-specific baseline drift was carried out by subtracting the measurements recorded for a sensor loaded without Fab. All assays were performed with agitation set to 1,000 r.p.m. at 30° C. Data analysis and curve fitting were carried out using the Octet analysis software, version 9.0. Experimental data were fitted using a 1:1 binding model for all experiments. Global analyses of the complete data sets, assuming binding was reversible (full dissociation), were carried out using nonlinear least-squares fitting allowing a single set of binding parameters to be obtained simultaneously for all concentrations used in each experiment.

For anti-HA IgGs, recombinant HAs (H1 A/Solomon Islands/3/2006, H3 A/Wisconsin/67/2005) (Whittle et al. Proc. Natl. Acad. Sci. USA 108, 14216-14221, 2011) were immobilized in separate channels by amine-coupling at pH 6.0. BSA was immobilized in the reference channel, to correct for buffer effects and non-specific binding signal. All SPR measurements were performed in HBS-EP running buffer (10 mM HEPES, pH 7.4, 150 mM NaCl, 3 mM EDTA, 0.005% vol/vol surfactant P20; GE Healthcare). Serially diluted antibodies were injected in triplicates at 30 μl/min for 2 minutes and allowed a dissociation time of 10 minutes. The chip was regenerated after each binding event with 50 mM Tris, pH 11.5, with a contact time of 15 seconds. The resulting sensorgrams were fitted with a two-state model (with conformation change) using Biaevaluation 3.0 software. The KD values reported are the average of the three technical replicates±s.d.

For EBOV GP_{Δmuc} antibody cross-competition assays, 10 μg/mL of GP_{Δmuc} was loaded onto Octet HTX biosensors using amine coupling (AR2G, FortéBiO) for 600 seconds. The newly identified antibodies, the KZ52 antibody as a positive control (Maruyama et al., J. Virol. 73, 6024-6030, 1999), and the isotype negative control (VRC01 (Wu et al., Science 329, 856-861, 2010) were diluted to 50 μg/mL in PBST-BSA. Binding of competitor and analyte monoclonal antibodies (mAbs) were each assessed for 1,800 seconds. The assay was performed in duplicate with agitation at 1,000 r.p.m. at 30° C. The percent inhibition (PI) was calculated by the equation: PI=100−[(probing mAb binding in the presence of competitor mAb)/(probing mAb binding in the absence of competitor mAb)]×100.

### Neutralization Assays

For EBOV neutralization, GP-pseudotyped lentiviral particles expressing a luciferase reporter gene were produced as described previously (Sullivan et al., PLoS Med. 3, 0865-0873, 2006), and were incubated 1 hour at 37° C. with serially diluted purified mAbs. HEK293T cells were infected with the lentivirus:mAb mixture for 72 hours in presence of polybrene (5 μg/ml, Sigma-Aldrich). Luciferase expression was assessed with Bright Glo (Promega) using a Victor X3 Plate Reader (PerkinElmer). Cell infection was calculated relative to the negative control antibody VRC01.

HIV-1 neutralization was assessed in TZM-b1 cells as described previously (Kong et al., Science 352, 828-833, 2016). Briefly, 293T cells were co-transfected by a pSG3AEnv backbone and an HIV-1 Env expression plasmid to produce Env-pseudotyped virus stocks. Viruses were mixed with fivefold serially diluted mAbs starting at 50 μg/ml, and incubated at 37° C. for 1 hour before being added to the cells. After incubation at 37° C. for 48 hours, the supernatants were removed and the cells were lysed. Luciferase activity was measured and 50% inhibitory concentrations (IC₅₀) were determined as described (Kong et al., Science 352, 828-833, 2016).

For flu neutralization, influenza pseudotyped lentiviral vectors expressing a luciferase reporter gene were produced as described (Yang et al., J. Virol. 78, 4029-4036, 2004). Briefly, the following plasmids: 17.5 μg pCMVΔR8.2, 17.5 μg pHRCMV-Luc, 0.3 μg pCMV Sport/h TMPRSS2, and 1 μg CMV/R-HA and 0.125 μg corresponding CMV/R-NA of a given strain of influenza virus were transiently co-transfected into 15-cm tissue culture plate of 293T cells using Fugene6 (Promega). Cells were transfected overnight and replenished with fresh medium. Forty-eight hours later, supernatants were harvested, filtered through a 0.45-μm PES (polyethersulfone) membrane filter, aliquoted, and frozen at −80° C. For neutralization assays, monoclonal antibodies at various dilutions were mixed with pseudoviruses and incubated at 37° C. for 1 hour before adding to 293A cells in 96-well plates (10,000 cells per well). Seventy-two hours later, cells were lysed in cell culture lysis buffer (Promega, Madison, Wis.) before mixing with luciferase assay reagent (Promega). Light intensity was quantitated with a PerkinElmer microplate reader and antibody neutralization results, in lower light intensity.

### Example 2: Functional Interrogation and Mining of Natively Paired Human VH:VL Antibody Repertoires

The human B-cell receptor repertoire constitutes an invaluable resource for discovery of therapeutic antibodies (Chan and Carter, Nat. Rev. Immunol. 10, 301-316, 2010; Brekke and Sandlie, Nat. Rev. Drug Discov. 2, 52-62, 2003). Cloning from individual B cells obtained via immortalization and expansion in vitro, or from single B cells obtained by limiting dilution or fluorescence-activated cell sorting (FACS), has been used extensively to discover anti-infective antibodies, including broadly neutralizing antibodies (bNAbs) to HIV-1 and influenza (Corti and Lanzavecchia, Annu. Rev. Immunol. 31, 705-742, 2013; Burton and Hangartner, Annu. Rev. Immunol. 34, 635-659, 2016). In parallel, over the last 25 years the screening of combinatorial libraries generated by random pairing of amplified VH and VL genes from human B cells has yielded numerous antibodies, leading to dozens of experimental or approved drug products (Bradbury et al., Nat. Biotechnol. 29, 245-254, 2011; Ecker et al., MAbs 7, 9-14, 2015). However, single-cell cloning is time- and resource-intensive, and is therefore limited to analysis of a small fraction of the human antibody repertoire (Wilson and Andrews, Nat. Rev. Immunol. 12, 709-719, 2012; Georgiou et al., Nat. Biotechnol. 32, 158-168, 2014), whereas combinatorial library screening has the capacity to interrogate antibody function from millions of B cells. However, the non-cognate pairing of VH and VL sequences in these libraries frequently gives rise to antibodies with lower selectivity and inferior biophysical properties compared to authentic human immunoglobulins (Jayaram et al., Protein Eng. Des. Sel. 25, 523-529, 2012; Ponsel et al., Molecules 16, 3675-3700, 2011).

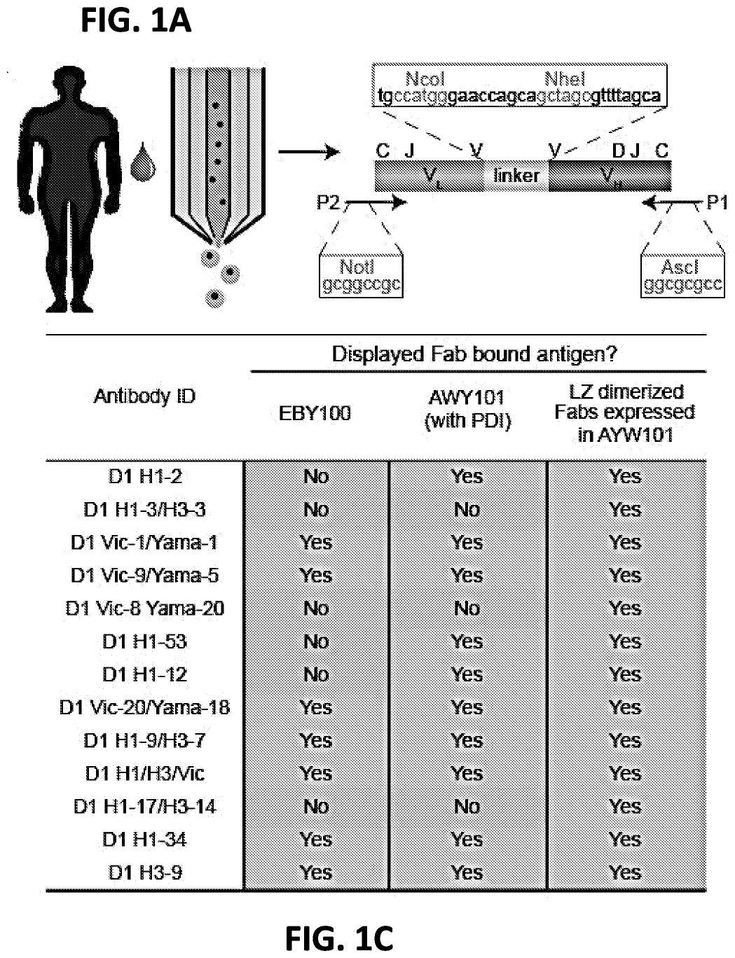

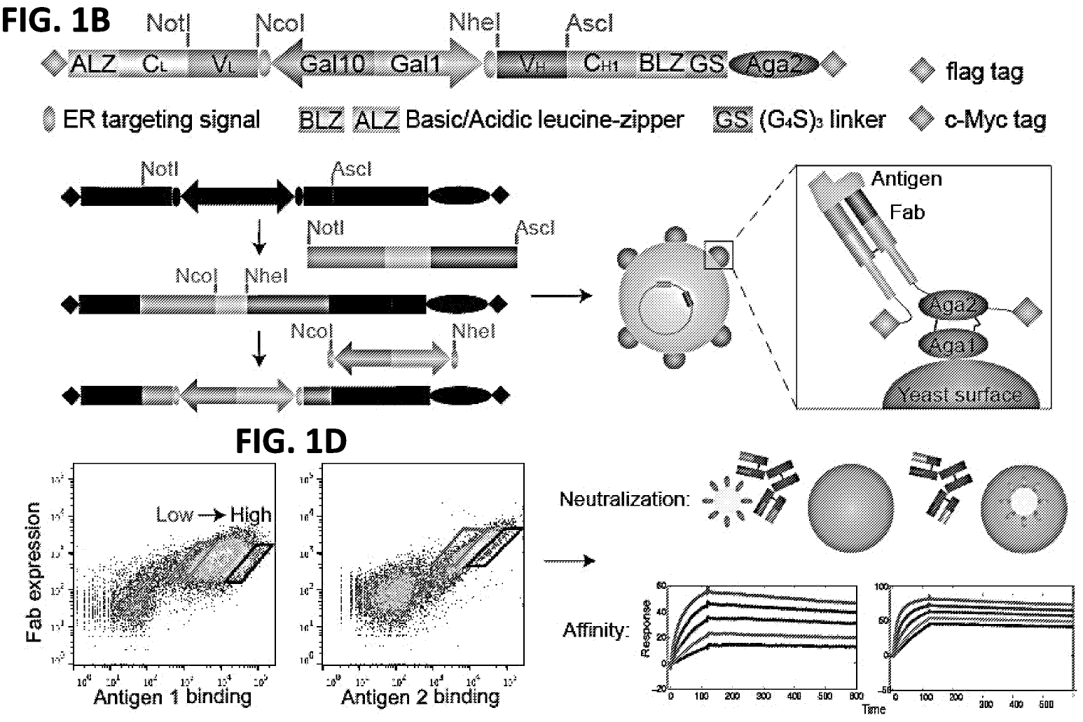

This example describes a technology for large-scale functional interrogation of the natively paired VH:VL antibody repertoire (FIG. 1). Because VH and VL genes are encoded by separate mRNA transcripts, they are first physically linked into a single amplicon for subsequent cloning into an expression vector. VH and VL linkage is accomplished by a two-step single-cell emulsion lysis and oligo-dT capture of VH and VL mRNAs from the same B cell, followed by a second reverse transcription (RT) and overlap-extension (OE)-PCR step to create contiguous VH:VL amplicons (DeKosky et al., Nat. Med. 21, 86-91, 2015; McDaniel et al., Nat. Protoc. 11, 429-442, 2016). In these amplicons the VH and VL genes are joined through a linker designed to enable one-step sub-cloning into a yeast Fab surface-expression vector, whereby the VH and VL genes are transcribed from a galactose-inducible bidirectional promoter with CH1 (human IgG1 isotype) and CL (human κ and λ2 isotypes) at the C terminus of the VH and VL, respectively (FIGS. 1A, 1B and 3; Table 1).

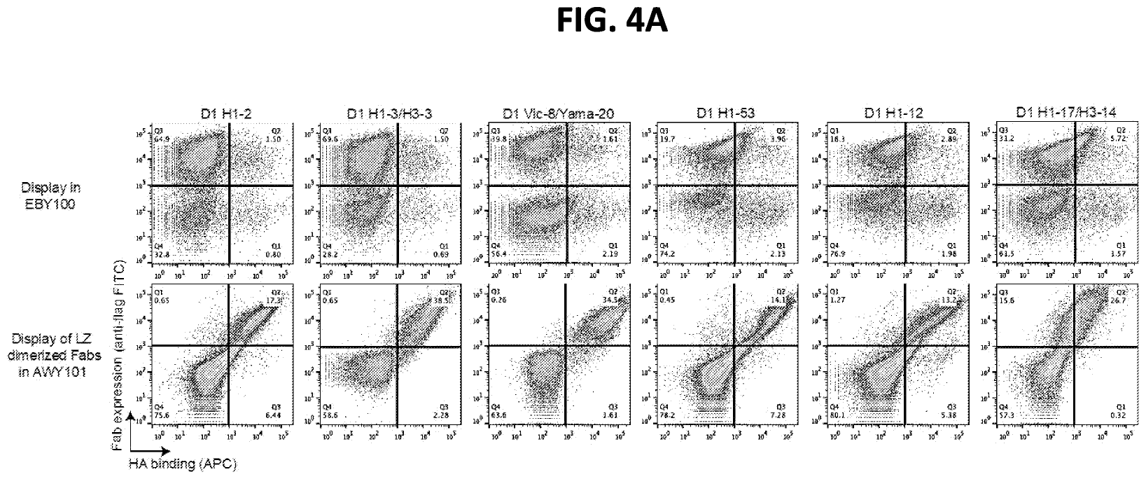

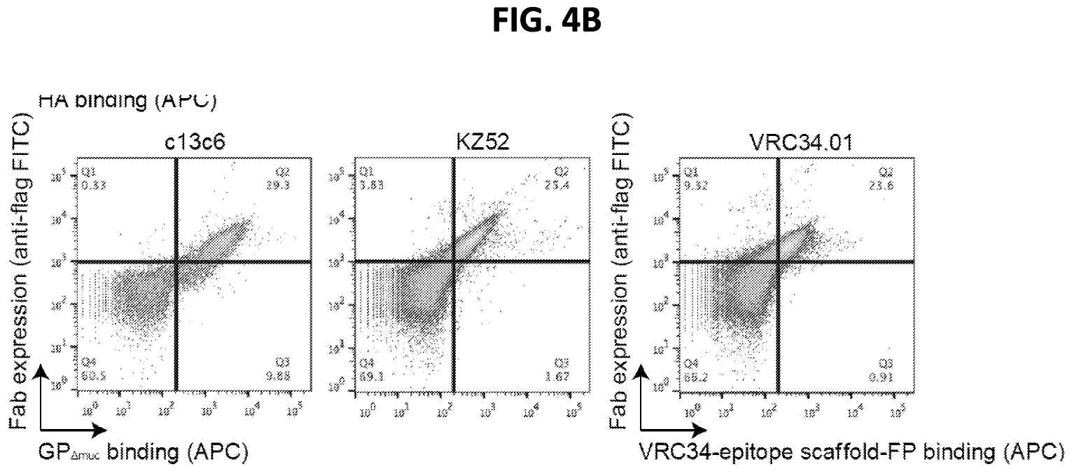

Human antibodies often express poorly in microbial hosts (Spadiut et al., Trends Biotechnol. 32, 54-60, 2014), and while expression efficiency in yeast is substantially higher relative to Escherichia coli or phage (Spadiut et al., Trends Biotechnol. 32, 54-60, 2014; Bowley et al., Protein Eng. Des. Sel. 20, 81-90, 2007; Feldhaus et al., Nat. Biotechnol. 21, 163-170, 2003), examination of a panel of 13 previously reported human influenza hemagglutinin (HA)-specific antibodies revealed that only 7/13 antibodies (53%) bound antigen when displayed on yeast (FIG. 1C and FIG. 4) (Lee et al., Nat. Med. 22, 1456-1464, 2016). Consistent with earlier reports (Wentz and Shusta, Appl. Environ. Microbiol. 73, 1189-1198, 2007), co-expression of protein disulfide isomerase (PDI) increased display efficiency, as monitored by two-color flow cytometry, to 10/13 antibodies (FIG. 1C). In addition to PDI expression, the enhanced dimerization of heavy and light chains via fusion to C-terminal leucine-zipper domains resulted in the display of the full set of 13/13 human anti-HA antibodies (FIG. 1C and FIG. 4) (Ojima-Kato et al., Protein Eng. Des. Sel. 29, 149-157, 2016). The display efficiency of three other human antibodies (two anti-Ebola virus (EBOV) antibodies: c13c6 and KZ52, and the anti-HIV-1 bNAb N123-VRC34.01 that targets the HIV-1 fusion peptide (Kong et al., Science 352, 828-833, 2016)) was tested. All three antibodies displayed efficiently and were shown to bind selectively to their respective antigens in the optimized system (FIG. 4). Extensive earlier studies demonstrated that yeast display enables interrogation of the human antibody repertoire based on affinities or off-rates, for epitope specificity, and for other properties including stability 15 (FIG. 1D). Clones of interest can then be expressed either as Fab or as IgG for detailed functional and biochemical assays (FIG. 1D).

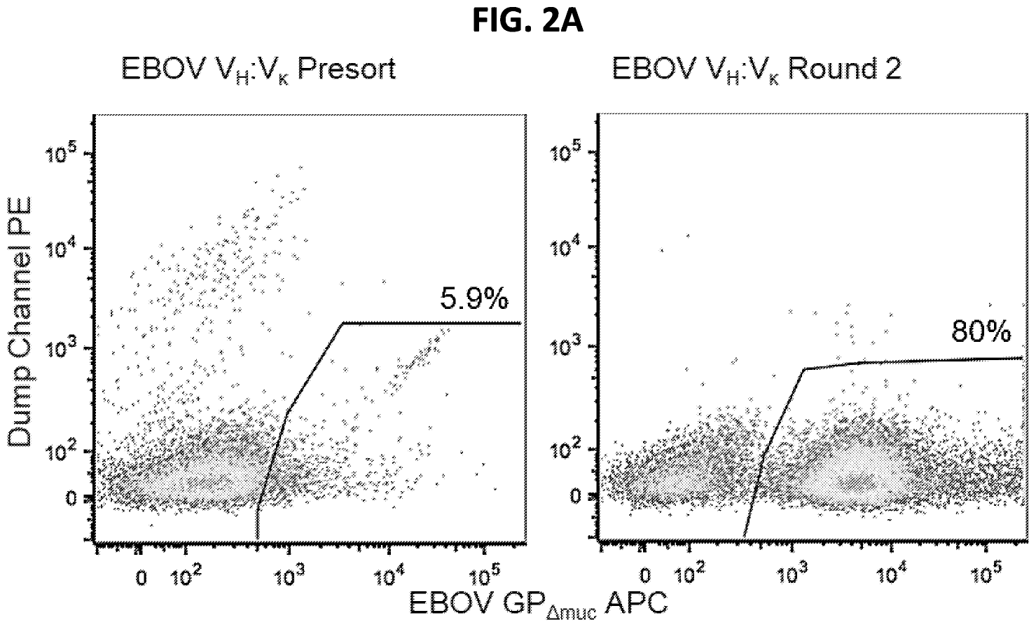

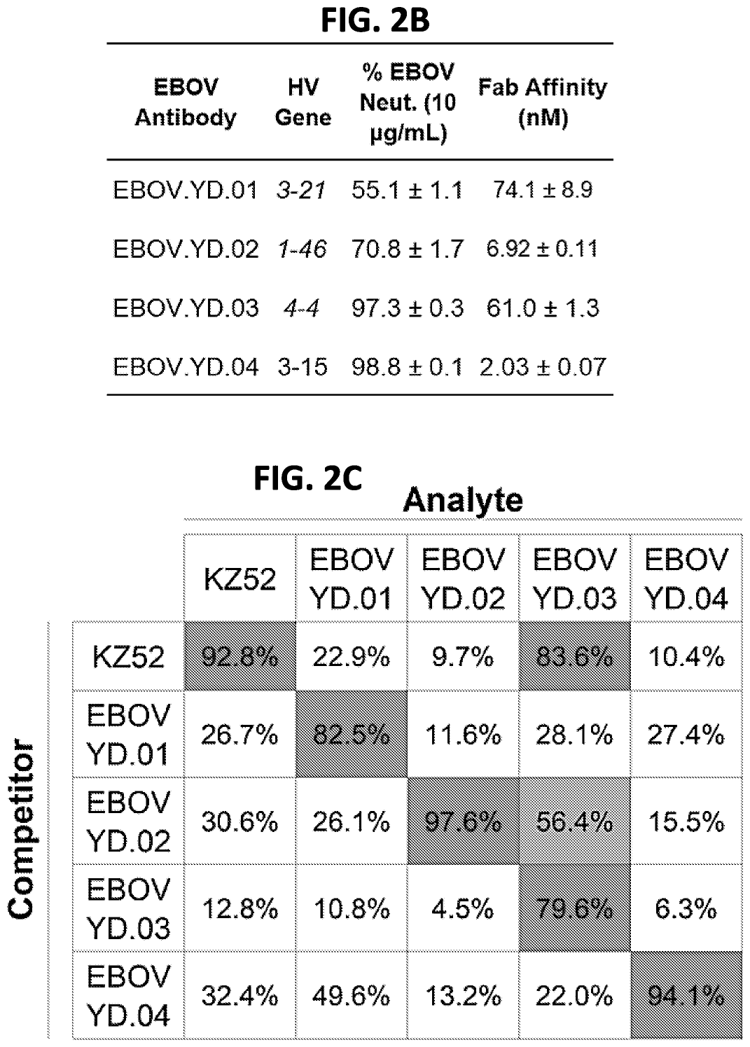

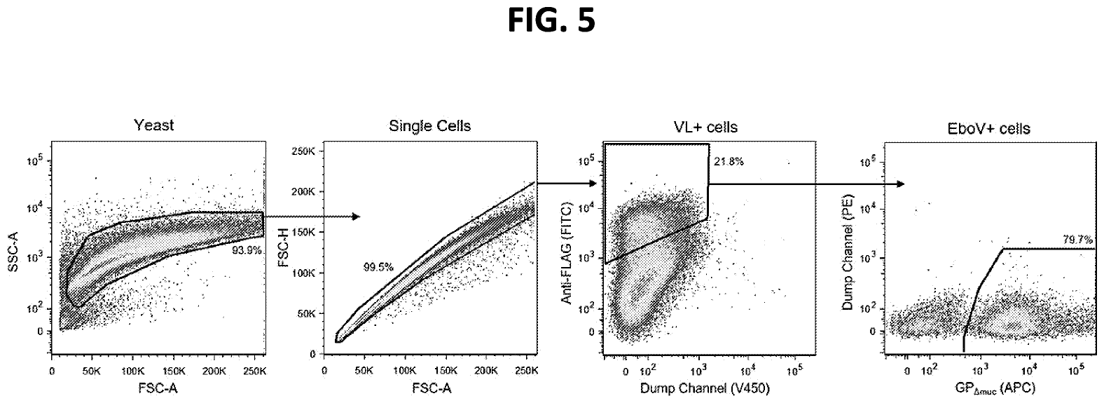

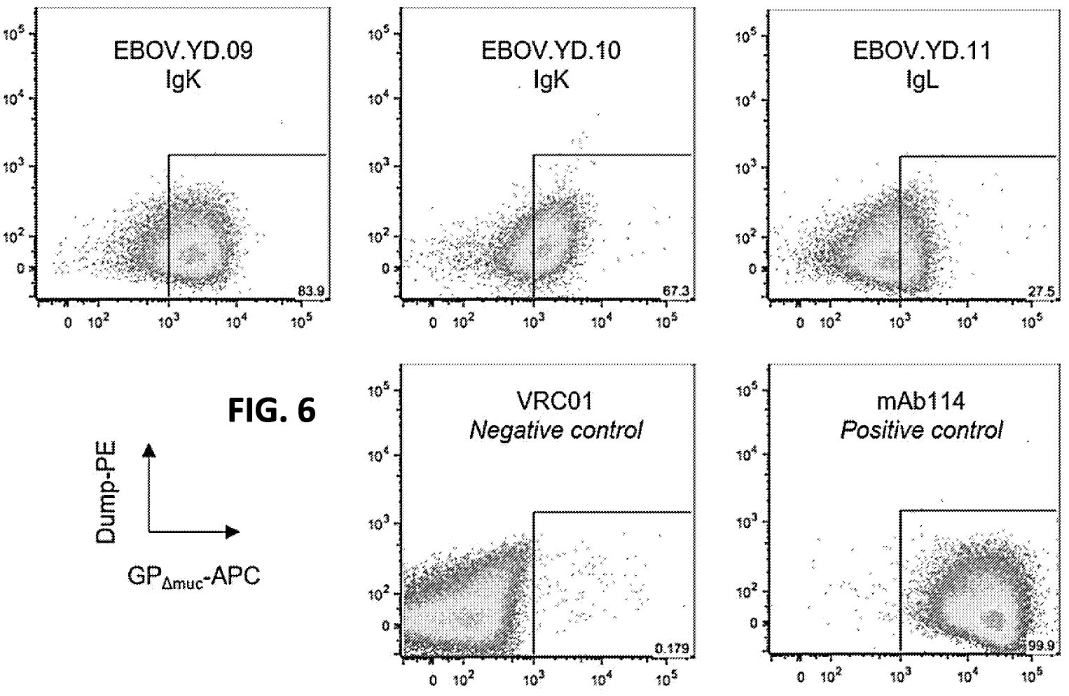

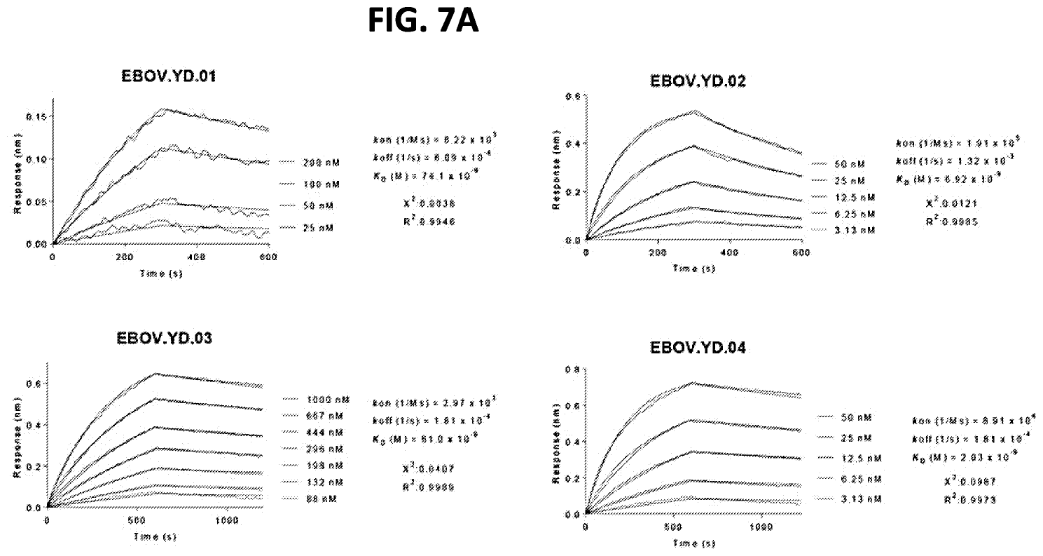

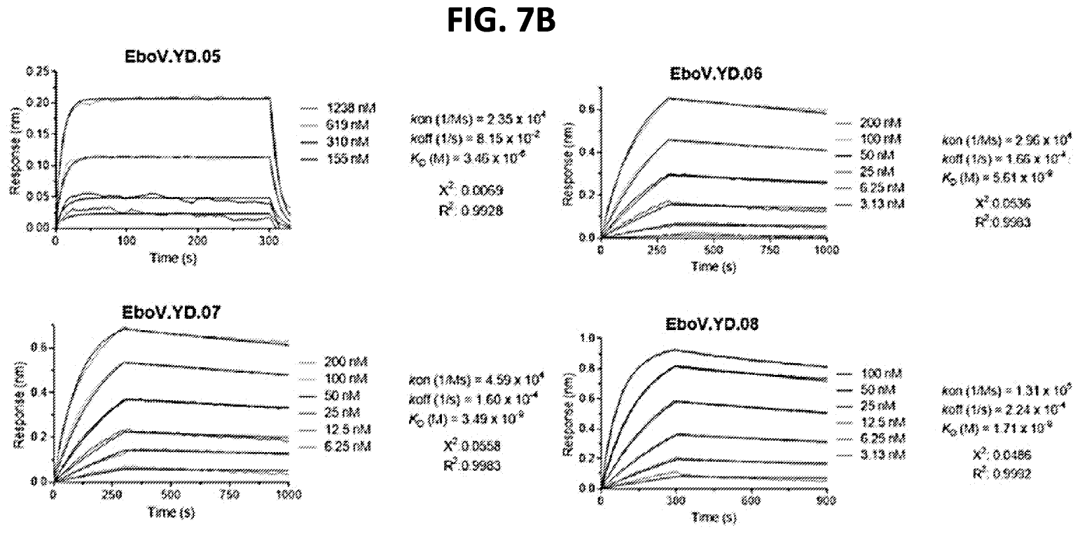

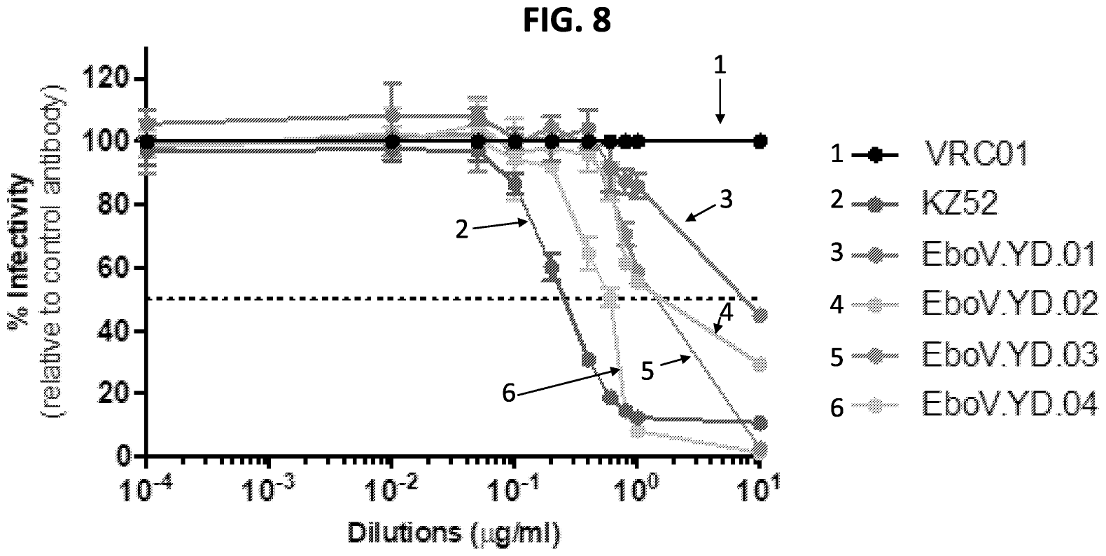

This approach was used to analyze the antibody repertoire of an individual 6 days after immunization with an experimental EBOV vaccine (Stanley et al., Nat. Med. 20, 1126-1129, 2014). This peak plasmablast VH:VL repertoire was displayed in yeast, and cells were analyzed and sorted for binding to EBOV mucin-like domain deleted glycoprotein (GP_{Δmuc}). High-throughput sequencing (HTS) was used to track antibody lineages throughout the screening process. Of 1,189 unique CDRH3:CDRL3 nucleotide clusters obtained from 5,002 plasmablasts after highly stringent sequence quality filtering, 828 were verified as cloned and displayed in the system using HTS (70% overall efficiency for library construction and display). As expected for the peak post-vaccination plasmablast response, an appreciable (6%) fraction of repertoire-expressing yeast cells in the pre-sort library bound to antigen, and antigen-specific clones were highly enriched after the third round of sorting (FIG. 2A and FIG. 5). Single-colony analysis of yeast yielded seven antibody lineages that bound to GP_{Δmuc} (EBOV.YD.01-EBOV.YD.04, EBOV.YD.09-EBOV.YD.11; Tables 2A and 2B and FIG. 6). Comparison of HTS data sets for the presort library and the sorted library after three rounds of screening revealed that all seven clones isolated above had been enriched by ≥120-fold. Four of these antibodies were randomly selected (EBOV.YD.01-EBOV.YD.04) and expressed as IgG1s in HEK293 cells, then digested to generate Fabs, which were shown to bind GP_{Δmuc} with nM affinities by biolayer interferometry (BLI) (FIG. 7A; Tables 2A and 2B). All four antibodies blocked infection by EBOV GP-pseudotyped lentiviral particles, with neutralization ranging from 55% to 99% at 10 μg/ml (FIG. 2B and FIG. 8). Competition assays revealed that these antibodies targeted distinct non-overlapping epitopes (FIG. 2C). EBOV.YD.03 competed with the well-characterized neutralizing antibody KZ52, indicating that it binds an epitope similar to antibodies generated during natural infection 21 (FIG. 2C).

TABLE 2A
Germline gene usage and HCDR3 sequences of anti-EBOV antibodies

| Antibody | HV Gene | HJ Gene | HCDR3 | Residues |
|---|---|---|---|---|
| EBOV.YD.01 | HV3-21 | HJ4 | CARENTIPFGGGVVLERA | 96-119 of SEQ ID NO: 2 |
| | | | SHFDYW | |
| EBOV.YD.02 | HV1-46 | HJ4 | CARDMHGVLSWYHALDYW | 96-113 of SEQ ID NO: 6 |
| EBOV.YD.03 | HV4-4 | HJ2 | CARIRVLPAAMLRGDYWY | 95-116 of SEQ ID NO: 10 |
| | | | FDLW | |
| EBOV.YD.04 | HV3-15 | HJ6 | CTTRVSIFRGPIEDVW | 98-113 of SEQ ID NO: 14 |
| EBOV.YD.05 | HV3-21 | HJ4 | CARDIGWAQPPGADYW | 111-126 of SEQ ID NO: 18 |
| EBOV.YD.06 | HV4-39 | HJ6 | CARFARFMTTSGDLIVSL | 97-122 of SEQ ID NO: 22 |
| | | | DYYAFDVW | |
| EBOV.YD.07 | HV3-15 | HJ4 | CVAHGDPVEAQW | 98-109 of SEQ ID NO: 26 |
| EBOV.YD.08 | HV1-2 | HJ5 | CARAVRGTTAVAGTWRFD | 96-115 of SEQ ID NO: 30 |
| | | | PW | |

TABLE 2B
Germline gene usage and LCDR3 sequences of anti-EBOV antibodies

| Antibody | LV Gene | LJ Gene | LCDR3 | Residues |
|---|---|---|---|---|
| EBOV.YD.01 | KV1-39 | KJ2 | CQQSYSAPYTF | 88-98 of SEQ ID NO: 4 |
| EBOV.YD.02 | KV1-39 | KJ5 | QQGYRIPIT | 89-97 of SEQ ID NO: 8 |
| EBOV.YD.03 | LV3-1 | LJ1 | CQAWDSSIGVF | 87-97 of SEQ ID NO: 12 |
| EBOV.YD.04 | LV1-40 | LJ1 | CQSYDSSLRDSYVF | 90-103 of SEQ ID NO: 16 |
| EBOV.YD.05 | KV1-9 | KJ1 | CQQVNSYPRTF | 88-98 of SEQ ID NO: 20 |
| EBOV.YD.06 | KV1-39 | KJ2 | CQQSYTTPRVTF | 88-99 of SEQ ID NO: 24 |
| EBOV.YD.07 | LV1-40 | JL3 | CQSYDSSLSDNWVF | 90-103 of SEQ ID NO: 28 |
| EBOV.YD.08 | LV1-51 | LJ2 | CGTWDSSLGAGVF | 89-101 of SEQ ID NO: 32 |

HTS surveillance of antibody clonal prevalence during screening enabled the retrieval of other antibodies that were enriched across rounds, but not identified by single-colony picking. Four additional antibody lineages that had been enriched more than eightfold in HTS data sets after multiple rounds of FACS were synthesized. Three out of four antibodies identified by HTS bound to GP_{Δmuc} with single-digit nM KD (EBOV.YD.06-EBOV.YD.08; FIG. 7B; Tables 2A and 2B) while another clone, EBOV.YD.05, bound weakly (˜3 μM KD as a Fab).

This yeast display technology was applied to assess an antibody lineage in an HIV-1-infected donor. Kong and co-workers recently identified N123-VRC34, an HIV-1 bNAb lineage that binds to the fusion peptide (FP) (Kong et al., Science 352, 828-833, 2016). The antibody repertoire of 1.42 million peripheral B cells from this donor (N123) were interrogated and the VH:VL repertoire was amplified with a unique human FR1 primer set that was also supplemented with lineage-specific primers. Only the inclusion of lineage-specific primers enabled successful recovery of the N123-VRC34 lineage, which contains several reported FR1 mutations and is extremely rare at this time point within the donor (roughly 0.003% of all B cells (Kong et al., Science 352, 828-833, 2016)). Yeast libraries were sorted using an epitope protein scaffold containing the eight terminal AA of the fusion peptide (VRC34-epitope scaffold-FP-APC) and a version of the scaffold alone without the fusion peptide (VRC34-epitope scaffold-KO-PE) (Kong et al., Science 352, 828-833, 2016).

HTS revealed that after three rounds of screening, VRC34-lineage antibodies far outcompeted other antibody lineages, constituting 98.7% of high-quality sequences and suggesting that the VRC34 lineage dominated the FP-specific repertoire in this donor. Three prevalent VH:Vκ clones were expressed and the respective Fabs were shown to bind to the HIV-1 fusion peptide probe with high affinity. To further “bin” FP-binding clones based on affinity, the yeast population was gated during the third round of sorting by increasing fluorescence intensity to FP; Fabs of four clones restricted to a high-, medium-, or low-affinity gated population had KD values consistent with their respective FACS profile. In total, seven unique VRC34 lineage antibodies were identified and all were broadly neutralizing Three double-nucleotide changes within a codon were observed that resulted in nonsynonymous amino acid substitutions, which are highly unlikely to have resulted from PCR or other artifacts and thus likely arose from somatic hypermutation in the donor, suggesting site-specific selection in vivo. These results suggest that genetic-lineage targeting coupled with yeast display can be useful for antibody discovery against HIV-1 or other difficult pathogens for which bNAbs are reported to have specific genetic requirements (Tian et al., Cell 166, 1471-1484. e18, 2016; Joyce et al., Cell 166, 609-623, 2016).

Finally, a paired VH:VL library was constructed from 12 million peripheral B cells harvested 270 days after immunization with seasonal, trivalent inactivated influenza vaccine (IIV3) (Moody et al., PLoS One 6, e25797, 2011) when HA-specific B cells occur at a frequency of ˜0.01% (Pinna et al., Eur. J. Immunol. 39, 1260-1270, 2009). Single yeast colonies were isolated after one round of sorting for antibody-expressing cells and four rounds of screening with Group 1 HA (18 clones) and separately, Group 2 HA (16 clones) included in IIV3 (Group 1: H1 from A/Solomon Islands/3/2006; Group 2: H3 from A/Wisconsin/67/2005); decreasing concentrations of antigen were used across rounds to increase selection stringency. Of these, 15/34 (44%) colonies encoded four unique antibody lineages to HA (one targeting H1 and three targeting H3) that bound to recombinant HAs with affinities ranging from 0.35 to 39.9 nM when expressed as IgGs; an additional 7/34 colonies (21%) recognized HAs but with lower affinities. Two of the four antibodies neutralized influenza with picomolar inhibitory concentrations (IC₅₀).

The display of a properly folded, functional antibody repertoire in yeast constitutes a renewable resource for the isolation of human antibodies and also for repeated analyses of the antibody response based on properties such as affinity, epitope coverage (such as by sorting in the presence of competitor antibodies), and stability (Feldhaus et al., Nat. Biotechnol. 21, 163-170, 2003). Yeast surface display has been reported to have a lower expression bias relative to other microbial display technologies (Spadiut et al., Trends Biotechnol. 32, 54-60, 2014; Bowley et al., Protein Eng. Des. Sel. 20, 81-90, 2007; Feldhaus et al., Nat. Biotechnol. 21, 163-170, 2003), and the yeast display optimization reported here further ensured bona fide expression of human antibody repertoires. Native VH:VL antibodies are expected to show superior selectivity and biophysical properties compared to randomly paired VH and VL antibodies isolated using other display platforms (Jayaram et al., Protein Eng. Des. Sel. 25, 523-529, 2012; Ponsel et al., Molecules 16, 3675-3700, 2011). Native antibody libraries displayed on yeast can be screened for antigens that bind to B-cell surface ligands (for example, sialic acid (Whittle et al., J. Virol. 88, 4047-4057, 2014) or CR2 (Kanekiyo et al., Cell 162, 1090-1100, 2015)) and are therefore not suitable for single-B-cell sorting, and also can be used to discover antibodies targeting insoluble antigens, including membrane proteins (Tillotson et al., Methods 60, 27-37, 2013; Wang et al., Nat. Methods 4, 143-145, 2007; Fang et al., MAbs 9, 1253-1261, 2017).

### Example 3: Ebola Virus-Specific Monoclonal Antibodies

This example describes the characterization of eight EBOV-specific monoclonal antibodies.

In Vitro and In Vivo Assessment of EBOV mAbs

To determine mAb in vitro functionality, including reactivity with the Ebola virus surface glycoprotein GP, mAbs were tested for binding to mucin-domain-deleted GP by yeast display (YD) and biolayer interferometry (BLI). Global mapping to determine mAb binding properties and epitopes on GP was performed by evaluation by BLI of competition with other mAbs that have known epitopes. Affinity to the Ebola GP for mAbs was determined using BLI. Neutralization of virus infection by mAbs was determined using pseudotyped lentivirus particles bearing the Ebola glycoprotein. Infection caused by the viruses was determined by measuring the expression of a luciferase reporter gene that is encoded by the virus genome.

Selection of EBOV mAbs by Yeast Display

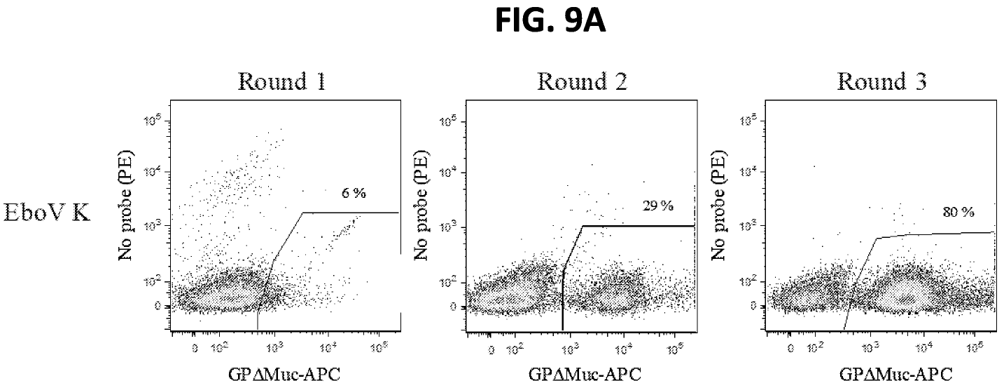

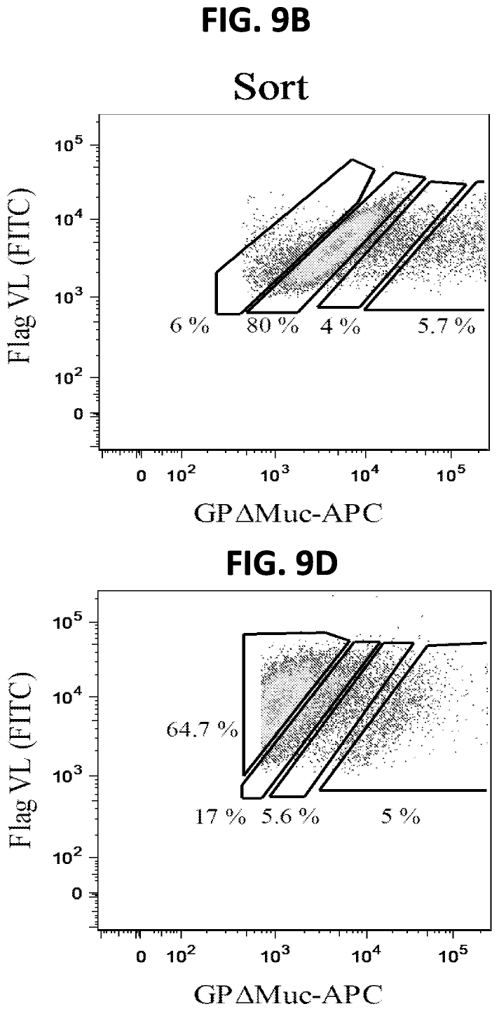

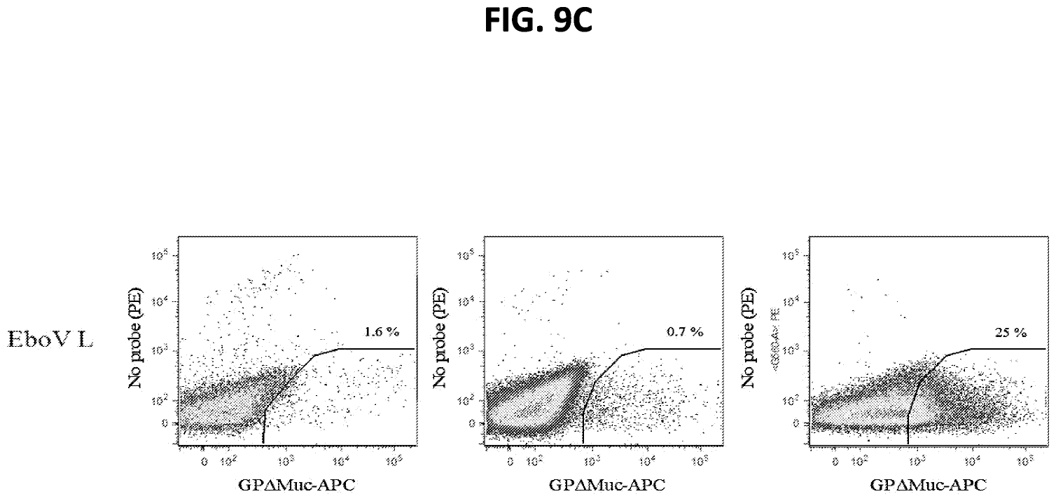

The variable domains of mAbs EboV.YD.01-EboV.YD.08 were isolated through paired sequencing of the heavy and light chain immunoglobulin genes from day 7 post-boost plasmablasts from a chimp adenovirus 3 (chAd3) prime and modified vaccinia virus Ankara (MVA) Ebola virus GP encoding vaccine recipient (see Examples 1 and 2). Isolated and paired variable heavy (VH) and variable light (VL) chain sequencing was performed. Amplicons from this sequencing were used in the creation of immunoglobulin plasmid libraries that were subsequently used to transform yeast. The resultant yeast display (YD) libraries expressed antigen binding fragments (Fabs) of the immunoglobulins expressed by the plasmablasts. Since <6% of the Day 7 post-boost plasmablasts YD library bound to the mucin-deleted GP (GPΔMuc), three rounds of selective enrichment were performed to increase binding >12.5 fold and decrease the likelihood of false positives (FIGS. 9A and 9C). Round three yeast were sorted into four groups that were determined by comparing the surface Fab expression levels to the amount of GPΔMuc probe bound (FIGS. 9B and 9D). This approach was chosen because it correlates with the relative affinity of the Fabs. Individual candidate yeast were selected from each sorted population using limiting dilution.

Ebola virus mAb EboV.YD.01

The nucleotide and amino acid sequences for the VH and VL domains of the expressed version of EboV.YD.01 are shown below. CDR amino acid sequences are underlined.

EboV.YD.01 VH domain nucleic acid sequence
(SEQ ID NO: 1)
CAGGTGCGGCTGGTGCAATCTGGGGGAGGCCTGGTCAAGCCTGGGGGGTC
CCTGAGACTCTCCTGTGCAGCCTCTGGATTCACCTTCAGCACCTTTGCCA
TGCATTGGGTCCGCCAGGCTCCAGGGAAGGGGCTGGAGTGGGTCTCATCC
ATTAGTCGTAGTAGTGGTTCCATAAACTACGCAGACTCAGTGAAGGGCCG
ATTCACCATCTCCAGAGACAACGCCAAGAACTCACTGTTTCTGCAAATGA
ACAGCCTGAGAGCCGACGACACGGCTGTCTATTACTGTGCGCGAGAGAAC
ACGATTCCGTTTGGGGGAGGTGTCGTCCTTGAAAGGGCATCACACTTTGA
CTACTGGGGCCAGGGAACCACGGTCACCGTCTCTTCA
EboV.YD.01 VH domain amino acid sequence
(SEQ ID NO: 2)
QVRLVQSGGGLVKPGGSLRLSCAASGFTFSTFAMHWVRQAPGKGLEWVSS
ISRSSGSINYADSVKGRFTISRDNAKNSLFLQMNSLRADDTAVYYCAREN
TIPFGGGVVLERASHFDYWGQGTTVTVSS
EboV.YD.01 VL domain nucleic acid sequence
(SEQ ID NO: 3)
GACATCCGGGTGACCCAGTCTCCATCCTCCCTGTCTGCATCTGTAGGAGA
CAGAGTCACCATCATTTGCCGGGCAAGTCAGAGCAGTAGTACTTTCCTAA
ATTGGTATCAGCAGAAACCAGGGAAAGCCCCTAACCTCCTGATCTACGCT
GCATCCAGTTTGCAAAGTGGGGTCCCATCAAGGTTCAGTGGCAGTGGATC
TGGGACAGATTTCACTCTCACCATCAGCAGTCTGCAACCAGAAGATTTTG
CAACTTACTACTGTCAACAGAGTTACAGTGCCCCGTACACTTTTGGCCAG
GGGACCAAAGTGGATATCAAA
EboV.YD.01 VL domain amino acid sequence
(SEQ ID NO: 4)
DIRVTQSPSSLSASVGDRVTIICRASQSSSTFLNWYQQKPGKAPNLLIYA
ASSLQSGVPSRFSGSGSGTDFTLTISSLQPEDFATYYCQQSYSAPYTFGQ
GTKVDIK

| EboV.YD.01 | CDR1 | CDR2 | CDR3 |
|---|---|---|---|
| VH domain (SEQ ID NO: 2) | 26-33 | 51-58 | 96-119 |
| VL domain (SEQ ID NO: 4) | 27-32 | 50-52 | 88-98 |

### Binding of EboV.YD.01 to EBOV GPΔMuc

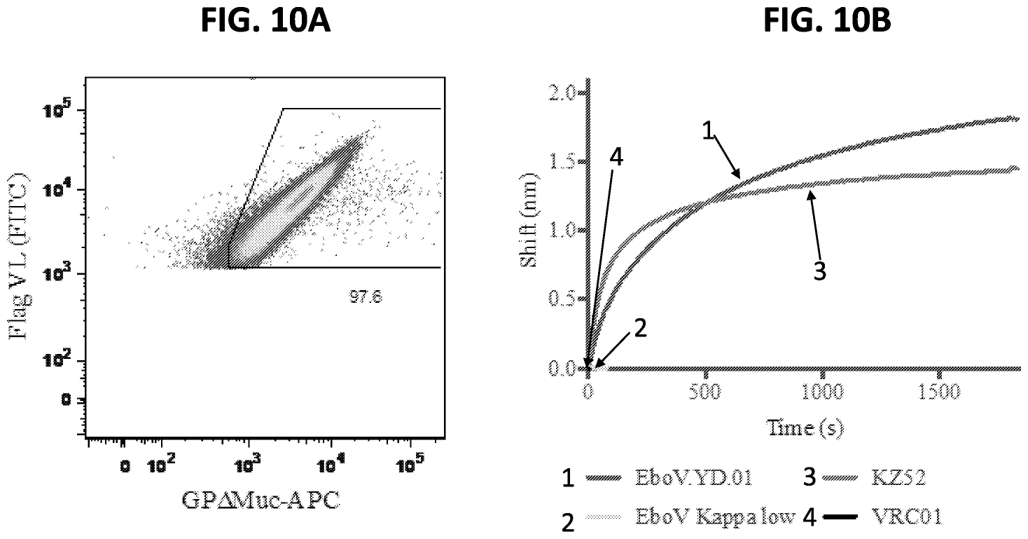

Single colony yeast expressing EboV.YD.01 was derived from sorted gates and reprobed for binding to GPΔMuc using flow cytometry to confirm its expressed Fab bound to GPΔMuc (FIG. 10A). Binding of the full EboV.YD.01 IgG mAbs to GPΔMuc was confirmed using biolayer interferometry (FIG. 10B).

### Epitope Mapping

By assessing how EboV.YD.01 competes with previously characterized mAbs, gross epitope was determined. Competition class was determined using BLI. Briefly, biosensors were loaded with purified mucin-domain-deleted GP. The competitor mAb (the mAb determining the class or gross epitope) was then allowed to bind to the antigen and the degree of binding was recorded. Then the analyte mAb was allowed to bind and the degree of binding was recorded. Percent Inhibition of the binding of the analyte was calculated as follows:

$$\% Inhibition = 100 ( 1 - \frac{signal of analyte binding in the presence of competitor}{signal of analyte binding in the absence of competitor} )$$

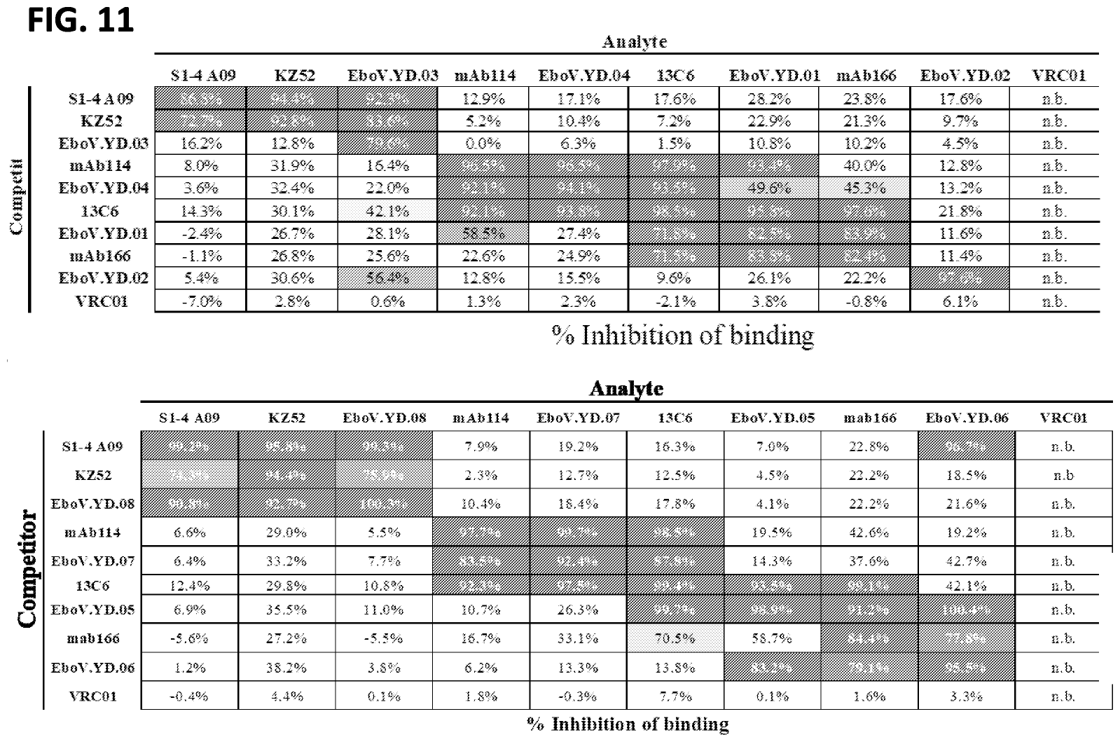

The assay puts EboV.YD.01 in the same competition class as mAb166 and 13C6, which are antibodies which bind in the glycan cap of GP (Misasi et al., Science 351(6279): 1343-1346, 2016; Lee et al., Nature 454(7201): 177-182, 2008). In contrast to mAb166, EboV.YD.01 shows moderate-to-high asymmetric competition with mAb114. Taken together, this suggests an epitope in the glycan cap (FIG. 11) that is unique from 13C6 and mAb166.

### EboV.YD.01 Kinetics of Binding to EBOV GPΔMuc

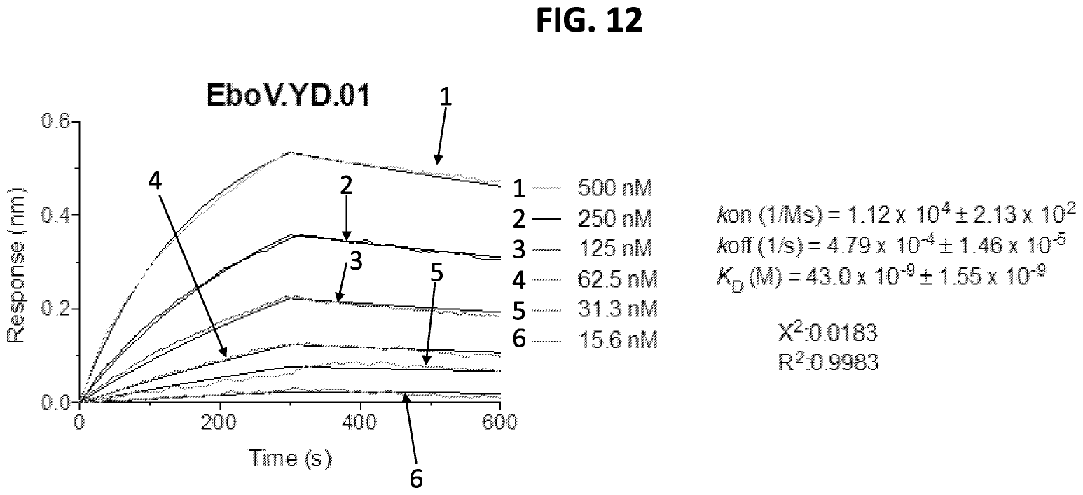

Fab protein generated from EboV.YD.01 IgG was evaluated for binding to EBOV GPΔMuc using BLI. EboV.YD.01 Fab showed binding to EBOV GPΔMuc with an affinity constant (KD) of 43 nM, kₒₙ of 1.13×10⁴ per second and k_{off} of 4.79×10⁻⁵ per Molar•second (FIG. 12).

### EboV.YD.01 Neutralization

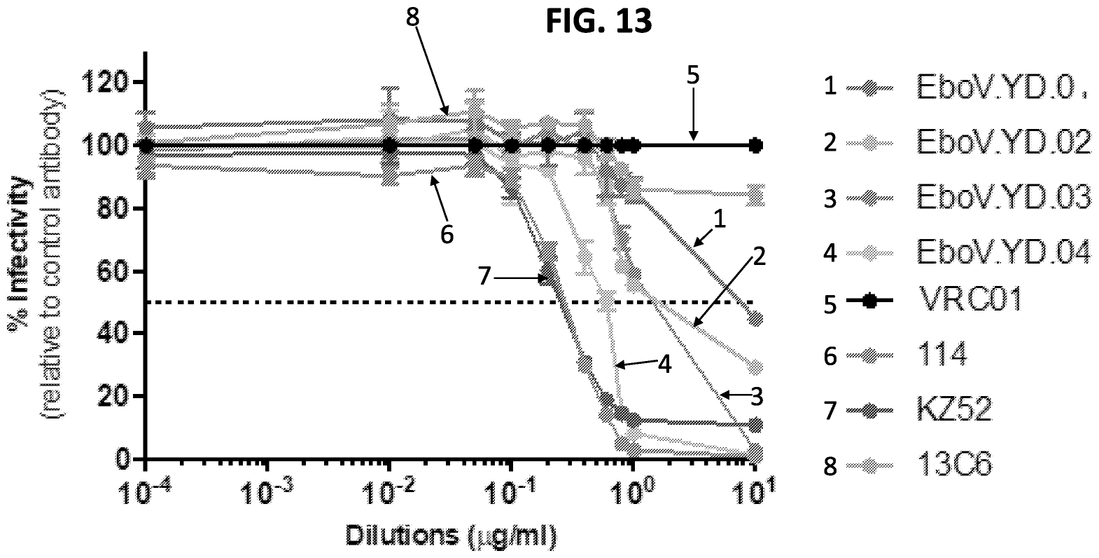

EboV.YD.01 was tested for neutralization activity in a pseudotyped virus entry assay. EboV.YD.01 was found to have an IC50 of 7.1 μg/ml. The neutralization potency of this antibody is unexpectedly high in view of its affinity (43 nM). This is an improvement over the prototypic glycan cap-binding antibody 13C6, which does not show neutralization (FIG. 13).

### Ebola Virus mAb EboV.YD.02

The nucleotide and amino acid sequences for the VH and VL domains of the expressed version of EboV.YD.02 are shown below.

EboV.YD.02 VH domain nucleic acid sequence
(SEQ ID NO: 5)
CAGGTGCAGCTGGTGCAGTCTGGAGCAGAGGTGAAAAAGCCTGGGACCTC
AGTGAAAATTTCCTGCAAGGCATCTGGATACAGCTTCACCAGCAAGTATA
TGCACTGGGTGCGACAGGCCCCTGGACAAGGGCTTGAGTGGATGGGAAAA
ATCAACCCTAGTGGTGGTAGAAGAGACTACGCACAGAAGTTCCAGGGCAG
AGTCACCATGACCAGGGACACGTCCACGAGCACAGTCTACATGGAGCTGA
GCAGCCTGAGATCTGGGGACACGGCCGTCTATTACTGTGCGAGAGATATG
CACGGTGTGTTAAGCTGGTACCATGCCCTTGACTACTGGGGCCAGGGAAC
CCTGGTCACCGTCTCCTCA
EboV.YD.02 VH domain amino acid sequence
(SEQ ID NO: 6)
QVQLVQSGAEVKKPGTSVKISCKASGYSFTSKYMHWVRQAPGQGLEWMGK
INPSGGRRDYAQKFQGRVTMTRDTSTSTVYMELSSLRSGDTAVYYCARDM
HGVLSWYHALDYWGQGTLVTVSS
EboV.YD.02 VL domain nucleic acid sequence
(SEQ ID NO: 7)
GACATCCGGGTGACCCAGTCTCCATCCTCCCTGTCTACGTCTGTGGGAGA
CAGAGTCACCATCACTTGCCGGGCAAGTCAAGACATTGGTAGATATCTAA
ATTGGTATCAGCAGAAACCAGGGAAAGCCCCTAAGCTCCTGATCTATGGT
GCATCCAGTTTGCAAAGTGGGGTCCCATCAAGGTTCAGTGGCAGTGGATC
TGGGACAGATTTCACTCTCACCATCAGCAGTCTGCAACCTGAAGATTTTG
CAACTTACTACTGTCAACAGGGTTACCGCATCCCGATCACCTTCGGCCAA
GGGACACGACTGGAGATTAAA
EboV.YD.02 VL domain amino acid sequence
(SEQ ID NO: 8)
DIRVTQSPSSLSTSVGDRVTITCRASQDIGRYLNWYQQKPGKAPKWYGAS
SLQSGVPSRFSGSGSGTDFTLTISSLQPEDFATYYCQQGYRIPITFGQGT
RLEIK

| EboV.YD.02 | CDR1 | CDR2 | CDR3 |
|---|---|---|---|
| VH domain (SEQ ID NO: 6) | 26-33 | 51-58 | 96-113 |
| VL domain (SEQ ID NO: 8) | 27-32 | 50-52 | 89-97 |

### Binding of EboV.YD.02 to EBOV GPΔMuc

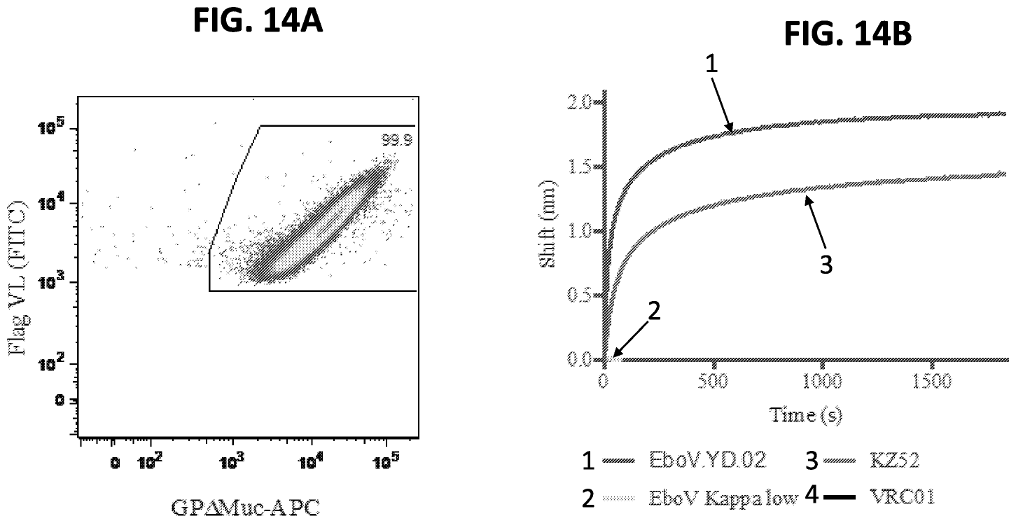

Single colony yeast expressing EboV.YD.02 was derived from sorted gates and reprobed for binding to GPΔMuc using flow cytometry to confirm its expressed Fab bound to GPΔMuc (FIG. 14A). Binding of the full EboV.YD.02 IgG mAbs to GPΔMuc was confirmed using biolayer interferometry (FIG. 14B).

### Epitope Mapping

Gross epitope determination via BLI competition assay was determined as described above (FIG. 11). The assay indicates that EboV.YD.02 does not significantly compete with any of the major competition groups and suggests that its epitope is unique (Misasi et al., Science 351(6279): 1343-1346, 2016; Lee et al., Nature 454(7201): 177-182, 2008).

### EboV.YD.02 Kinetics of Binding to EBOV GPΔMuc

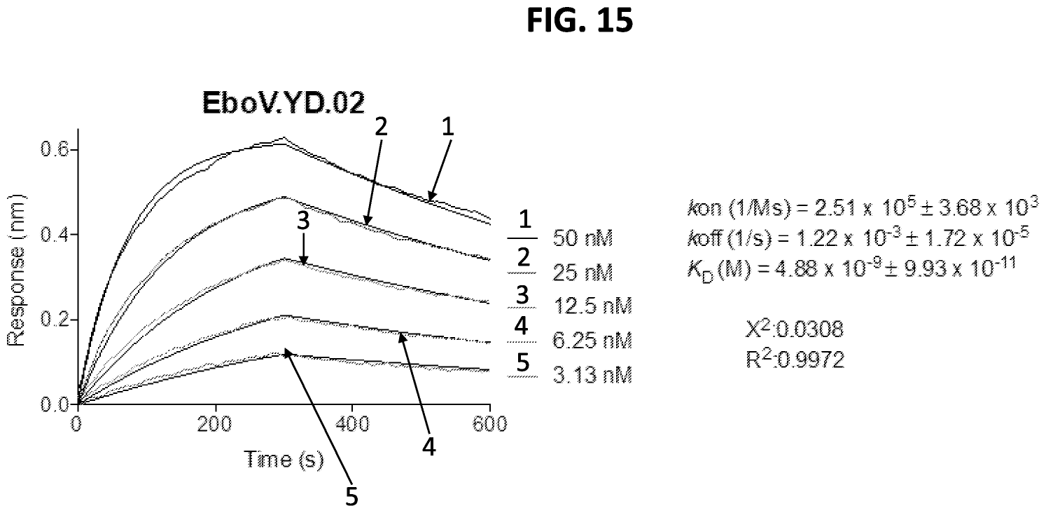

Fab protein generated from EboV.YD.02 IgG was evaluated for binding to EBOV GPΔMuc using BLI. EboV.YD.02 Fab showed binding to EBOV GPΔMuc with an affinity constant (KD) of 4.9 nM, kₒₙ of 2.51×10⁵ per second and k_{off} of 1.2×10⁻³ per Molar•second (FIG. 15)

### EboV.YD.02 Neutralization

EboV.YD.02 was tested for neutralization activity in a pseudotyped virus entry assay (FIG. 13). EboV.YD.02 was found to have an IC50 of 1.6 μg/ml. This is an improvement over 13C6 which does not show neutralization.

Ebola virus mAb EboV.YD.03

The nucleotide and amino acid sequences for the VH and VL domains of the expressed version of EboV.YD.03 are shown below.

EboV.YD.03 VH domain nucleic acid sequence
(SEQ ID NO: 9)
CAGGTGCAGCTGCAGGAGTCGGGCCCAGGACTAGTGAAGCCTTCGGAGAC
CCTGTCCCTCACCTGCACTGTCTCTGGTGACTCCCTCAGTAGTTCGTACT
GGAGCTGGATCCGGCAGTCCGCCGGGAAGGGACTGGAGTACATTGGGCGT
ACCTATGTTAGTGGGAACACCAAGTACAACCCCTCCCTCAAGAGTCGAGT
CACCATGTCAGTAGACACGTCCAAGAACCAGTTCTCCCTGAGACTGACCT
CTGTGACCGCCGCGGACACGGCCGTATATTACTGTGCGAGAATACGAGTG
CTACCAGCTGCTATGCTTAGAGGGGACTACTGGTACTTCGATCTCTGGGG
CCGTGGCACCCTGGTCACTGTCTCCTCA
EboV.YD.03 VH domain amino acid sequence
(SEQ ID NO: 10)
QVQLQESGPGLVKPSETLSLTCTVSGDSLSSSYWSWIRQSAGKGLEYIGR
TYVSGNTKYNPSLKSRVTMSVDTSKNQFSLRLTSVTAADTAVYYCARIRV
LPAAMLRGDYWYFDLWGRGTLVTVSS
EboV.YD.03 VL domain nucleic acid sequence
(SEQ ID NO: 11)
TCCTATGAGCTGACGCAGCTACCCTCAGTGTCCGTGTCACCAGGACAGAC
AGCGAGCATCACCTGCTCTGGAGATAAAGTGGAAAATAAATATGTTTGCT
GGTATCAGCAGAAGTCAGGCCAGTCCCCTGTCCTGGTCATCTATGAAGAT
AGTAAGCGGCCCTCAGGGATCCCTGAGCGATTCTCTGGCTCCAACTCTGG
AAACACAGCCACTCTGACCATCAGCGGGACCCAAACTATGGATGAGGCTG
ACTATTTCTGTCAGGCGTGGGACAGTAGTATTGGGGTCTTCGGAACTGGG
ACCAAGCTCACCGTCCTA
EboV.YD.03 VL domain amino acid sequence
(SEQ ID NO: 12)
SYELTQLPSVSVSPGQTASITCSGDKVENKYVCWYQQKSGQSPVLVIYED
SKRPSGIPERFSGSNSGNTATLTISGTQTMDEADYFCQAWDSSIGVFGTG
TKLTVL

| EboV.YD.03 | CDR1 | CDR2 | CDR3 |
|---|---|---|---|
| VH domain (SEQ ID NO: 10) | 26-33 | 51-57 | 95-116 |
| VL domain (SEQ ID NO: 12) | 26-31 | 49-51 | 87-97 |

### Binding of EboV.YD.03 to EBOV GPΔMuc

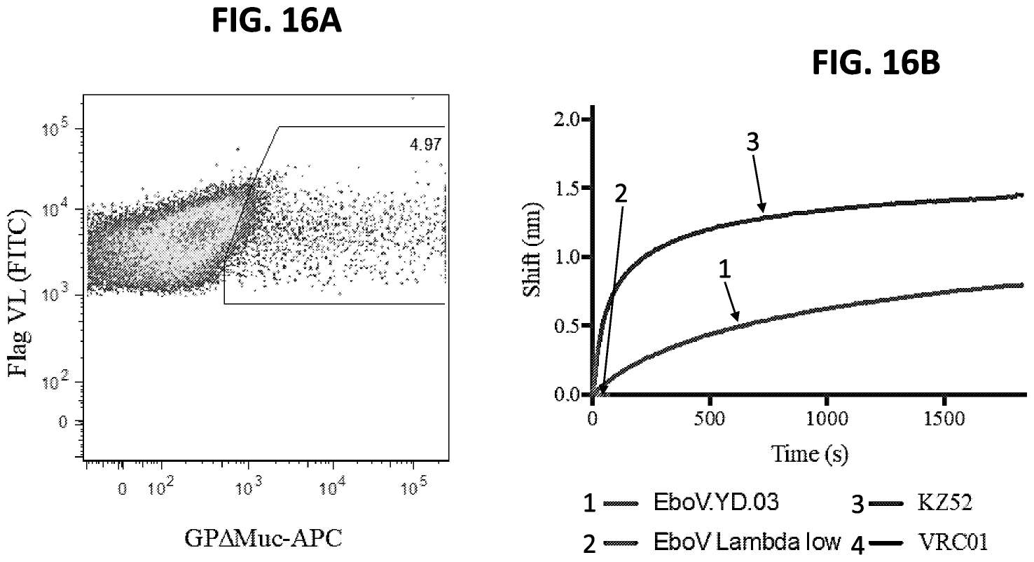

Single colony yeast expressing EboV.YD.03 was derived from sorted gates and reprobed for binding to GPΔMuc using flow cytometry to confirm its expressed Fab bound to GPΔMuc (FIG. 16A). Binding of the full EboV.YD.03 IgG mAbs to GPΔMuc was confirmed using biolayer interferometry (FIG. 16B).

### Epitope Mapping

Gross epitope determination via BLI competition assay was determined as described above (FIG. 11). The assay showed that both KZ52 and S1-4 A09 block binding of EboV.YD.03 but EboV.YD.03 does not block the binding of either KZ52 or S1-4 A09. This “asymmetric” competition suggests that the epitope is near the base of GP and is unique (Misasi et al., Science 351(6279): 1343-1346, 2016; Lee et al., Nature 454(7201): 177-182, 2008).

### EboV.YD.03 Kinetics of Binding to EBOV GPΔMuc

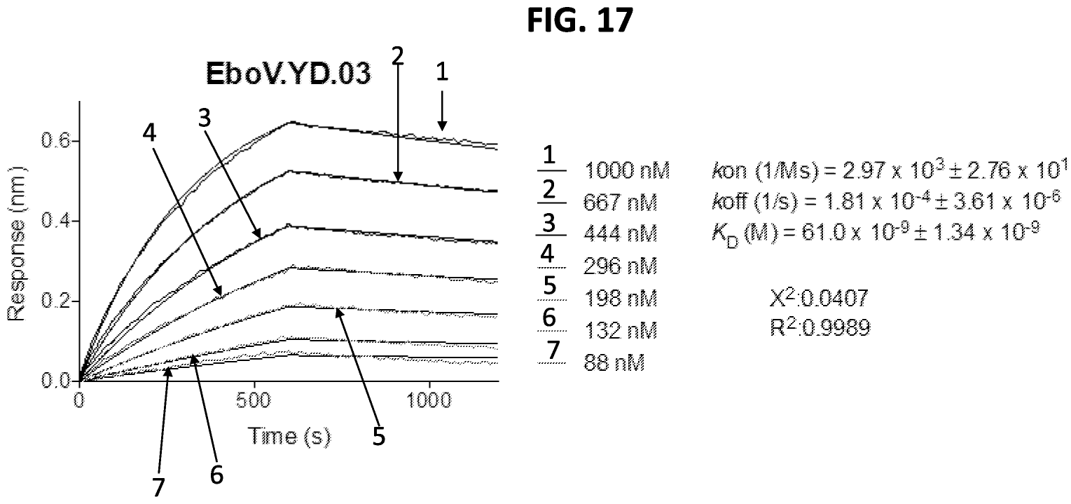

Fab protein generated from EboV.YD.03 IgG was evaluated for binding to EBOV GPΔMuc using BLI. EboV.YD.03 Fab showed binding to EBOV GPΔMuc with an affinity constant (KD) of 61 nM, kₒₙ of 2.97×10³ per second and k_{off} of 1.81×10⁻⁴ per Molar•second (FIG. 17).

### EboV.YD.03 Neutralization

EboV.YD.03 was tested for neutralization activity in a pseudotyped virus entry assay (FIG. 13). EboV.YD.03 showed near 100% neutralization at 10 μg/ml, with an IC50 of 1.1 μg/ml. This is an improvement over KZ52, which does not neutralize completely and 13C6 which does not show neutralization.

### Ebola Virus mAb EboV.YD.04

The nucleotide and amino acid sequences for the VH and VL domains of the expressed version of EboV.YD.04 are shown below.

EboV.YD.04 VH domain nucleic acid sequence
(SEQ ID NO: 13)
CAGGTGCAGCTGGTGGAGTCTGGGGGAGGCTTGGTAAAGCCTGGGGGGTC
CCTTAGACTCTCCTGTGCAGCCTCTGGATTCATGTTCAGTAATGCCTGGA
TGAACTGGGTCCGCCAGGCTCCAGGGAAGGGGCTGGAGTGGGTTGGACGT
ATTAAAAGCAGAAGTGATGGTGGGACAACAGACTACGCTGCACCCGTGAA
AGGCAGATTCACCTTCTCAAGAGAGGATTCAAAAAACATGCTGTATCTGC
AAATGAACAGCCTGAAACGCGAGGACACAGCCGTCTATTACTGTACCACC
AGAGTTTCTATTTTTCGGGGACCTATTGAGGACGTCTGGGGCCAAGGGAC
CACGGTCACCGTCTCCTCA
EboV.YD.04 VH domain amino acid sequence
(SEQ ID NO: 14)
QVQLVESGGGLVKPGGSLRLSCAASGFMFSNAWMNWVRQAPGKGLEWVGR
IKSRSDGGTTDYAAPVKGRFTFSREDSKNMLYLQMNSLKREDTAVYYCTT
RVSIFRGPIEDVWGQGTTVTVSS
EboV.YD.04 VL domain nucleic acid sequence
(SEQ ID NO: 15)
CAGCCTGTGCTGACTCAGCCGCCCTCAGTGTCTGGGGCCCCAGGGCAGAG
GGTCACCATCTCCTGCGCTGGAAGCAGCTCCAACATCGGGGCAGGTTATG
ATGTATACTGGTACCAGCAGCTTCCAGGAACTGCCCCCAAACTCCTCATC
TATGGAAACAACAATCGGCCCTCAGGGGTCCCTGACCGATTCTCTGGCTC
CAAGTCTGGCACCTCAGCCTCCCTGGCCATCACAGGGCTCCAGGCTGAGG
ATGAGGCTGAATATTACTGCCAGTCCTATGACAGCAGCCTGCGTGATTCT
TATGTCTTCGGAAGTGGGACCAAGGTGACCGTCCTA
EboV.YD.04 VL domain amino acid sequence
(SEQ ID NO: 16)
QPVLTQPPSVSGAPGQRVTISCAGSSSNIGAGYDVYWYQQLPGTAPKLLI
YGNNNRPSGVPDRFSGSKSGTSASLAITGLQAEDEAEYYCQSYDSSLRDS
YVFGSGTKVTVL

| EboV.YD.04 | CDR1 | CDR2 | CDR3 |
|---|---|---|---|
| VH domain (SEQ ID NO: 14) | 26-33 | 51-60 | 98-113 |
| VL domain (SEQ ID NO: 16) | 26-34 | 52-54 | 90-103 |

### Binding of EboV.YD.04 to EBOV GPΔMuc

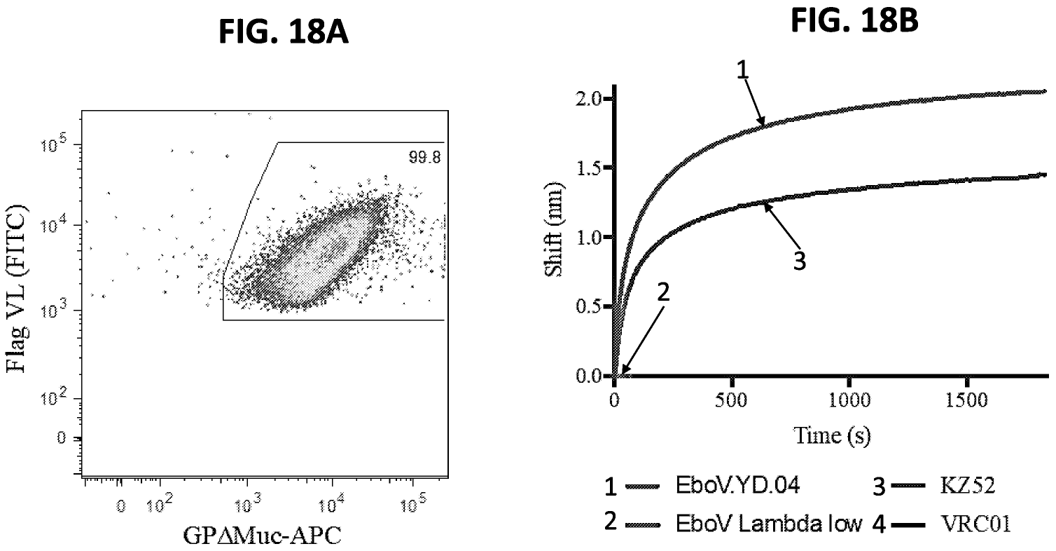

Single colony yeast expressing EboV.YD.04 was derived from sorted gates and reprobed for binding to GPΔMuc using flow cytometry to confirm its expressed Fab bound to GPΔMuc (FIG. 18A). Binding of the full EboV.YD.04 IgG mAbs to GPΔMuc was confirmed using biolayer interferometry (FIG. 18B).

### Epitope Mapping

Gross epitope determination via BLI competition assay was determined as described above (FIG. 11). The assay puts EboV.YD.04 in the same competition class as mAb114 and 13C6, which are antibodies that bind in the glycan cap of GP (Misasi et al., Science 351(6279): 1343-1346, 2016; Lee et al., Nature 454(7201): 177-182, 2008), suggesting an epitope in the glycan cap or GP₁ core.

### EboV.YD.04 Kinetics of Binding to EBOV GPΔMuc

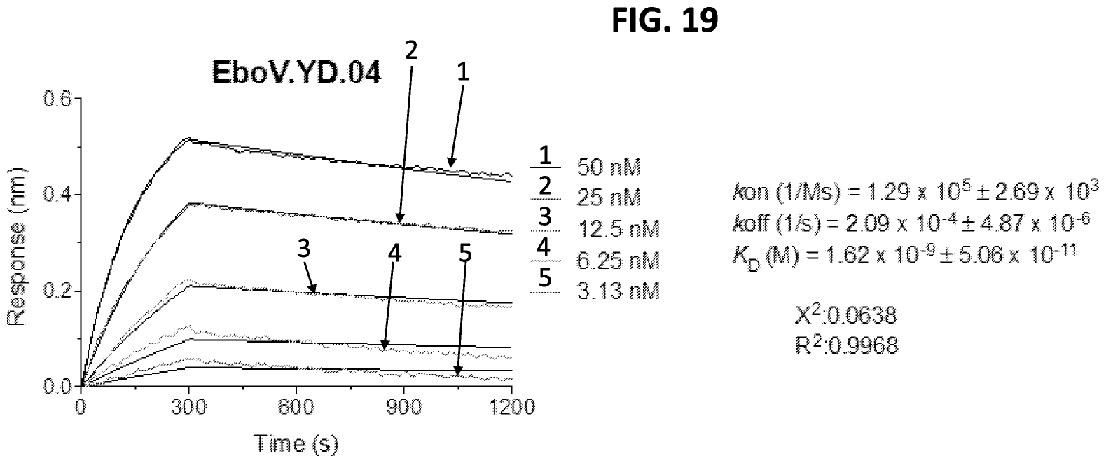

Fab protein generated from EboV.YD.04 IgG was evaluated for binding to EBOV GPΔMuc using BLI. EboV.YD.04 Fab showed binding to EBOV GPΔMuc with an affinity constant (KD) of 1.62 nM, kₒₙ of 1.25×10⁵ per second and k_{off} of 2.09×10⁻⁴ per Molar•second (FIG. 19).

### EboV.YD.04 Neutralization

EboV.YD.04 was tested for neutralization activity in a pseudotyped virus entry assay (FIG. 13). EboV.YD.04 shows near 100% neutralization at 10 μg/ml, with an IC50 of 0.5 μg/ml. This is similar to mAb114 and represents an improvement 13C6 which does not show neutralization.

### Ebola Virus mAb EboV.YD.05

The nucleotide and amino acid sequences for the VH and VL domains of the expressed version of EboV.YD.05 are shown below.

EboV.YD.05 VH domain nucleic acid sequence
(SEQ ID NO: 17)
GAGGTCCAGCTGGTGGAGTCTGGGGGAGGCCTGGTCAAGCCTGGGGGGTC
CCTGAGACTCTCCTGTGCAGCCTCTGGATTTACCCTCAGTAGTTATAGCA
TGAACTGGGTCCGCCAGGCTCCAGGGAAGGGGCTGGAATGGGTCTCATCC
GTTACTAGTAGTGATGACAAATACTACGCAGGCCGATTTACCCTCTCTAC
AGGCAGCAGTGATGATAAATACTACGCAGACTCAGTGAGGGGCCGCTTTA
CCATCTCCAGAGACAACGCCAAGAATTCACTCTATCTGCAAATGAACAGC
CTGAGAGCCGAAGACACAGCTATATATTATTGTGCGAGGGATATTGGATG
GGCACAACCGCCTGGGGCTGACTACTGGGGCCAGGGAACCCTGGTCACCG
TCTCCTCA
EboV.YD.05 VH domain amino acid sequence
(SEQ ID NO: 18)
EVQLVESGGGLVKPGGSLRLSCAASGFTLSSYSMNWVRQAPGKGLEWVSS
VTSSDDKYYAGRFTLSTGSSDDKYYADSVRGRFTISRDNAKNSLYLQMNS
LRAEDTAIYYCARDIGWAQPPGADYWGQGTLVTVSSH
EboV.YD.05 VL domain nucleic acid sequence
(SEQ ID NO: 19)
GACATCCAGTTGACCCAGTCTCCATCCTCCCTGTCTGCATCTGTAGGAGA
CAGAGTCACCATCACTTGCCGGGCCAGTCAGGGCATTAGAAGTTATTTAG
CCTGGTATCAGCAAAAACCAGGGAAAGCCCCTAAGCTCCTGATCTATGCT
GCATCCACTTTGCAAAGTGGGGTCCCATCAAGGTTCAGCGGCAGTGGATC
TGGGACAGATTTCACTCTCACCATCAGCAGCCTGCAGCCTGAAGATTTTG
CAACTTATTACTGTCAACAGGTTAATAGTTACCCTCGGACTTTCGGCCAA
GGGACCAAGGTGGAAATCAAA
EboV.YD.05 VL domain amino acid sequence
(SEQ ID NO: 20)
DIQLTQSPSSLSASVGDRVTITCRASQGIRSYLAWYQQKPGKAPKLLIYA
ASTLQSGVPSRFSGSGSGTDFTLTISSLQPEDFATYYCQQVNSYPRTFGQ
GTKVEIK

| EboV.YD.05 | CDR1 | CDR2 | CDR3 |
|---|---|---|---|
| VH domain (SEQ ID NO: 18) | 26-33 | 51-73 | 111-126 |
| VL domain (SEQ ID NO: 20) | 27-32 | 50-52 | 88-98 |

### Binding of EboV.YD.05 to EBOV GPΔMuc

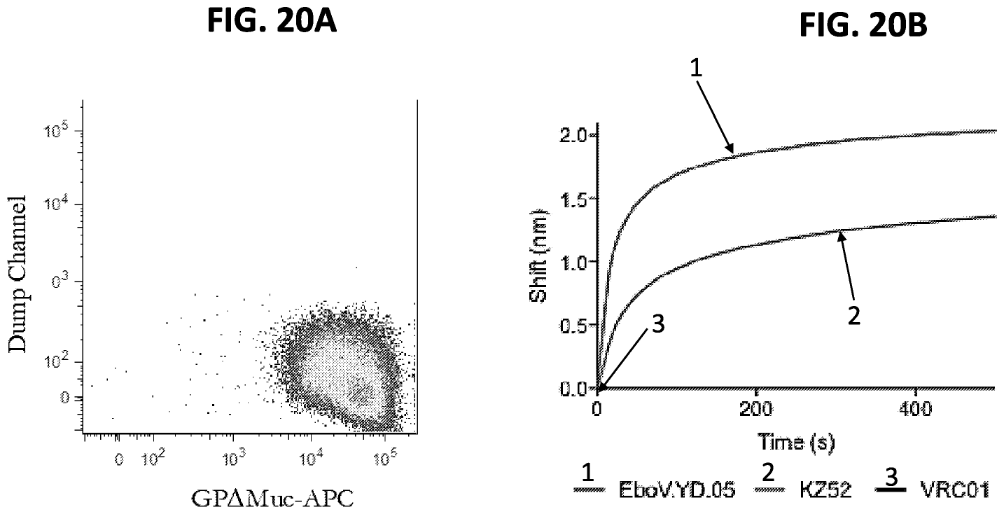

Single colony yeast expressing EboV.YD.05 was derived from sorted gates and reprobed for binding to GPΔMuc using flow cytometry to confirm its expressed Fab bound to GPΔMuc (FIG. 20A). Binding of the full EboV.YD.05 IgG mAbs to GPΔMuc was confirmed using biolayer interferometry (FIG. 20B).

### Epitope Mapping

Gross epitope determination via BLI competition assay was determined as described above (FIG. 11). The assay shows EboV.YD.05 competes with 13C6 for binding, but does not show the same competition profile as 13C6 (i.e., competition with itself, mAb114 and mAb166) (Misasi et al., Science 351(6279): 1343-1346, 2016; Lee et al., Nature 454(7201): 177-182, 2008). This suggests that EboV.YD.05 targets an epitope that is unique from 13C6 and other glycan cap antibodies.

### EboV.YD.05 Kinetics of Binding to EBOV GPΔMuc

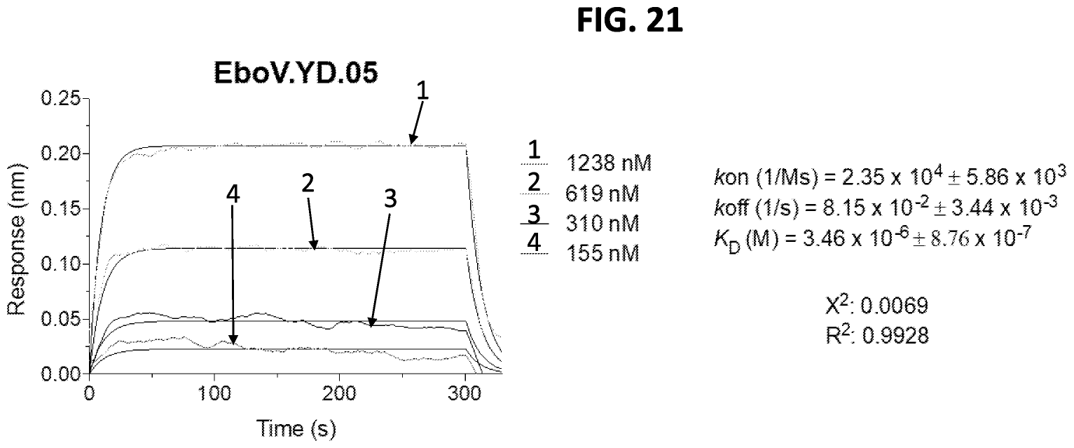

Fab protein generated from EboV.YD.05 IgG was evaluated for binding to EBOV GPΔMuc using BLI. EboV.YD.05 Fab showed binding to EBOV GPΔMuc with an affinity constant (KD) of 3.46 μM, kₒₙ of 2.35×10⁴ per second and k_{off} of 8.15×10⁻² per Molar•second (FIG. 21).

### Ebola Virus mAb EboV.YD.06

The nucleotide and amino acid sequences for the VH and VL domains of the expressed version of EboV.YD.05 are shown below.

EboV.YD.06 VH domain nucleic acid sequence
(SEQ ID NO: 21)
CAGCTGCAGCTGCAGGAGTCGGGCCCAGGACTGCTGAAGCCTTCGGAGAC
CCTGTCCCTCACTTGCAGTGTCTCTGGTGGCTCCATCAACAGTTATACTT
ACTACTGGGGCTGGGTCCGCCAGTCCCCAGCGAAGGGGCTGGAGTGGATT
GGGAGTTTCTCTTATAGTGGGAGTTCCCACTACAACCCGTCTCTTGAGAG
TCGAGTCACCATCTCCGTAGACAGGTCCAAGAATCAGGTCTCCCTGAAGC
TGAGTTCTGTGACCGCCGCAGACACGGCTGTGTATTACTGTGCGAGATTC
GCTAGGTTTATGACTACGTCTGGGGATCTTATCGTTAGTCTCGATTACTA
CGCTTTCGACGTCTGGGGCCAAGGGACCACGGTCACCGTCTCCTCA
EboV.YD.06 VH domain amino acid sequence
(SEQ ID NO: 22)
QLQLQESGPGLLKPSETLSLTCSVSGGSINSYTYYWGWVRQSPAKGLEWI
GSFSYSGSSHYNPSLESRVTISVDRSKNQVSLKLSSVTAADTAVYYCARF
ARFMTTSGDLIVSLDYYAFDVWGQGTTVTVSS
EboV.YD.06 VL domain nucleic acid sequence
(SEQ ID NO: 23)
GACATCCAGATGACCCAGTCTCCATCCTCCCTGTCTGCATCTGTAGGAGA
CAGAGTCACCATCACTTGCCGGGCAAGTCAGACCATTAGGAACAATTTAA
ATTGGTATCAGCAAAAACTAGGGAAAGCCCCTAAACTCCTGATCTATGCT
GCATCCACTTTACAAAATGGGGTCCCCTCGAGGTTCAGTGGCAGTGGATC
TGGGACAGATTTCACTCTCACCATCAGTAGTCTGCAACCTGAAGATTTTG
CGACTTACTATTGTCAACAGAGTTACACTACCCCTCGAGTCACTTTTGCC
CAGGGGACCAAGTTGGAGATCAAA
EboV.YD.06 VL domain amino acid sequence
(SEQ ID NO: 24)
DIQMTQSPSSLSASVGDRVTITCRASQTIRNNLNWYQQKLGKAPKLLIYA
ASTLQNGVPSRFSGSGSGTDFTLTISSLQPEDFATYYCQQSYTTPRVTFA
QGTKLEIK

| EboV.YD.06 | CDR1 | CDR2 | CDR3 |
|---|---|---|---|
| VH domain (SEQ ID NO: 22) | 27-35 | 53-59 | 97-122 |
| VL domain (SEQ ID NO: 24) | 27-32 | 50-52 | 88-99 |

### Binding of EboV.YD.06 to EBOV GPΔMuc

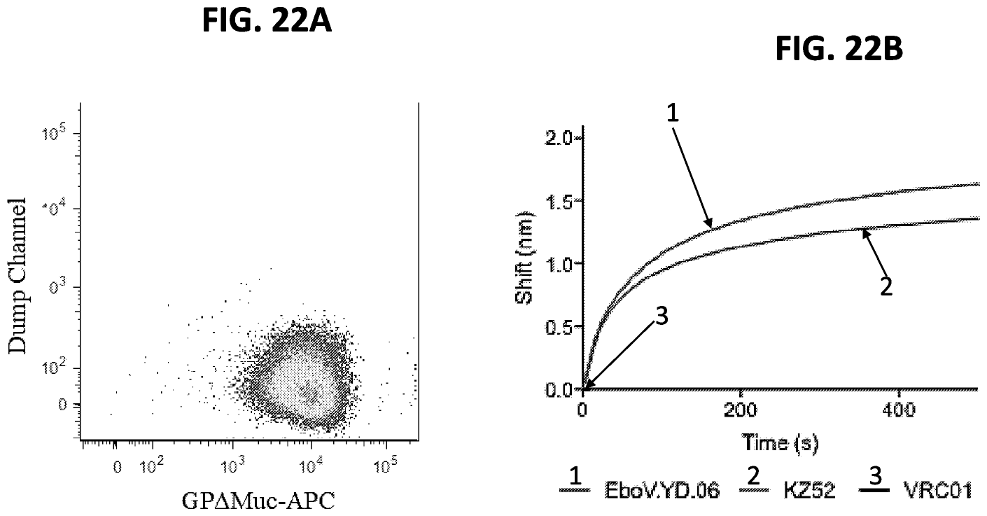

Single colony yeast expressing EboV.YD.06 was derived from sorted gates and reprobed for binding to GPΔMuc using flow cytometry to confirm its expressed Fab bound to GPΔMuc (FIG. 22A). Binding of the full EboV.YD.06 IgG mAbs to GPΔMuc was confirmed using biolayer interferometry (FIG. 22B).

### Epitope Mapping

Gross epitope determination via BLI competition assay was determined as described above. The assay show EboV.YD.06 competes with mAb166 and S1-4 A09 for binding to GP, (FIG. 11). Since it does not compete with other glycan cap mAbs (i.e., 13C6, mAb114) or base antibodies (i.e., KZ52), it suggests the epitope it targets an epitope near the glycan cap and GP base interface (Misasi et al., Science 351(6279): 1343-1346, 2016; Lee et al., Nature 454(7201): 177-182, 2008).

### EboV.YD.06 Kinetics of Binding to EBOV GPΔMuc

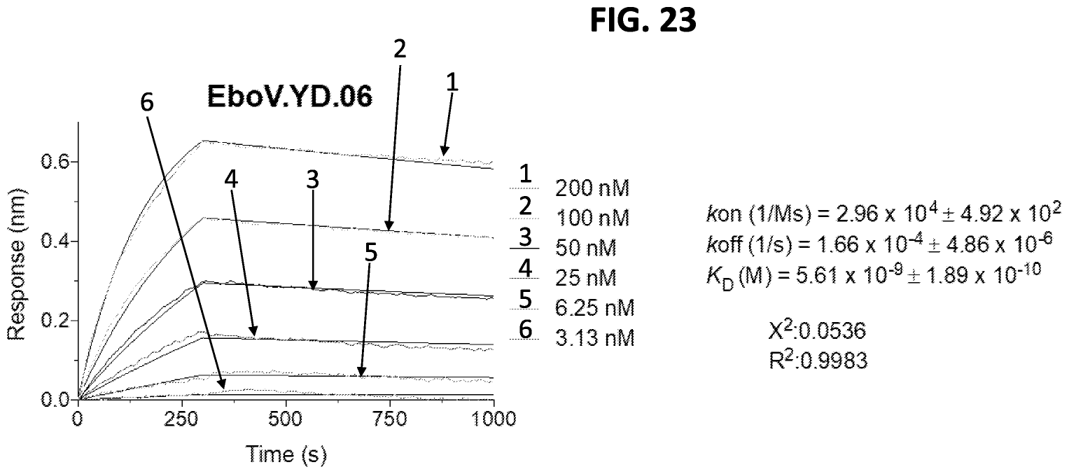

Fab protein generated from EboV.YD.06 IgG was evaluated for binding to EBOV GPΔMuc using BLI. EboV.YD.06 Fab showed binding to EBOV GPΔMuc with an affinity constant (KD) of 5.61 nM, kon of 2.96×10⁴ per second and k_{off} of 1.66×10⁴ per Molar•second (FIG. 23).

### Ebola Virus mAb EboV.YD.07

The nucleotide and amino acid sequences for the VH and VL domains of the expressed version of EboV.YD.07 are shown below.

EboV.YD.07 VH domain nucleic acid sequence
(SEQ ID NO: 25)
GAGGTCCAGCTGGTGGAGTCTGGGGGAGGCTTGGTGAAGCCTGGGGGGTC
CCTTAGACTCTCCTGTGCAGGCTCTGGATTCACTTTCACTAAAGCCTGGA
TGAGCTGGGTCCGCCAGGCTCCAGGGAAGGGGCTGGAGTGGGTTGGCCGT
ATTAAGAGCAGAGCTGATAGTGGGACAACAGCCTACACTGCACCCGTGAA
AGGCAGATTCACCATCTCAAGAGATGATTCAAAAAAGACGCTGTATCTGC
AAATGAACAGCCTGAAAACCGAGGACACAGCCGTGTACTATTGTGTCGCA
CATGGGGACCCAGTAGAGGCACAATGGGGCCAGGGAACCCTGGTCACCGT
CTCCTCT
EboV.YD.07 VH domain amino acid sequence
(SEQ ID NO: 26)
EVQLVESGGGLVKPGGSLRLSCAGSGFTFTKAWMSWVRQAPGKGLEWVGR
IKSRADSGTTAYTAPVKGRFTISRDDSKKTLYLQMNSLKTEDTAVYYCVA
HGDPVEAQWGQGTLVTVSS
EboV.YD.07 VL domain nucleic acid sequence
(SEQ ID NO: 27)
CAGTCTGTGCTGACTCAGCCGCCCTCAGTGTCTGGGGCCCCAGGGCAGAG
GGTCACCATCTCCTGCACTGGGGGCAGCTCCAACATCGGGGCAGGTTATG
ATGTACAATGGTACCAGCAGGTTCCAGGAACAGCCCCCAAACTCCTCATC
TATCATAACAACAATCGGCCCTCAGGGGTCCCTGACCGGTTCTCTGGCTC
CAAGTCTGGCACCTCAGCCTCCCTGGCCATCACTGGGCTCCAGGCTGAGG
ATGAGGCTGATTATTACTGCCAGTCTTATGACAGCAGCCTGAGTGACAAT
TGGGTGTTCGGCGGAGGGACCAAGCTGACCGTCCTA
EboV.YD.07 VL domain amino acid sequence
(SEQ ID NO: 28)
QSVLTQPPSVSGAPGQRVTISCTGGSSNIGAGYDVQWYQQVPGTAPKLLI
YHNNNRPSGVPDRFSGSKSGTSASLAITGLQAEDEADYYCQSYDSSLSDN
WVFGGGTKLTVL

| EboV.YD.07 | CDR1 | CDR2 | CDR3 |
|---|---|---|---|
| VH domain (SEQ ID NO: 26) | 27-33 | 51-60 | 98-109 |
| VL domain (SEQ ID NO: 28) | 26-34 | 52-54 | 90-103 |

### Binding of EboV.YD.07 to EBOV GPΔMuc

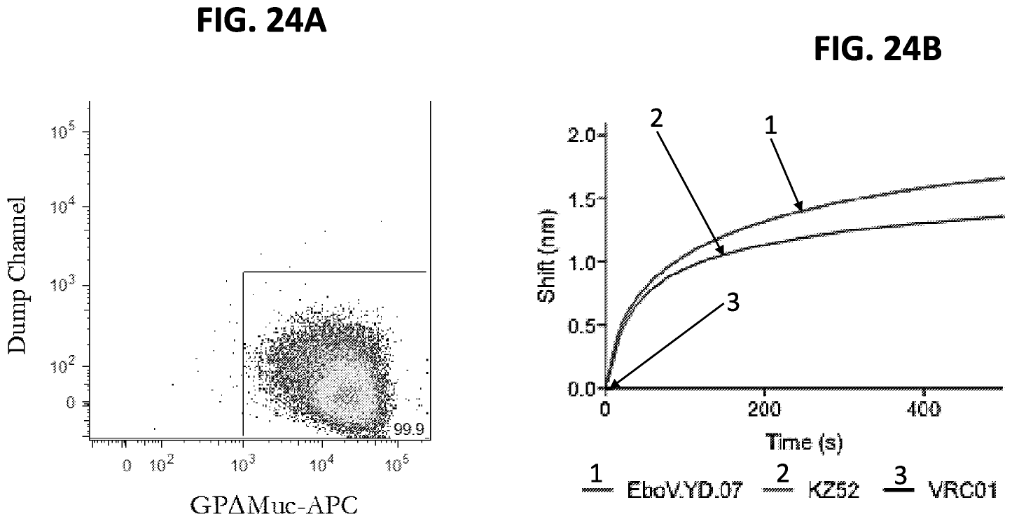

Single colony yeast expressing EboV.YD.07 was derived from sorted gates and reprobed for binding to GPΔMuc using flow cytometry to confirm its expressed Fab bound to GPΔMuc (FIG. 24A). Binding of the full EboV.YD.07 IgG mAbs to GPΔMuc was confirmed using biolayer interferometry (FIG. 24B).

### Epitope Mapping

Gross epitope determination via BLI competition assay was determined as described above (FIG. 11). The assay puts EboV.YD.07 in the same competition class as mAb114 and 13C6, which are antibodies which bind in the glycan cap of GP (Misasi et al., Science 351(6279): 1343-1346, 2016; Lee et al., Nature 454(7201): 177-182, 2008), suggesting an epitope in the glycan cap or GP₁ core.

### EboV.YD.07 Kinetics of Binding to EBOV GPΔMuc

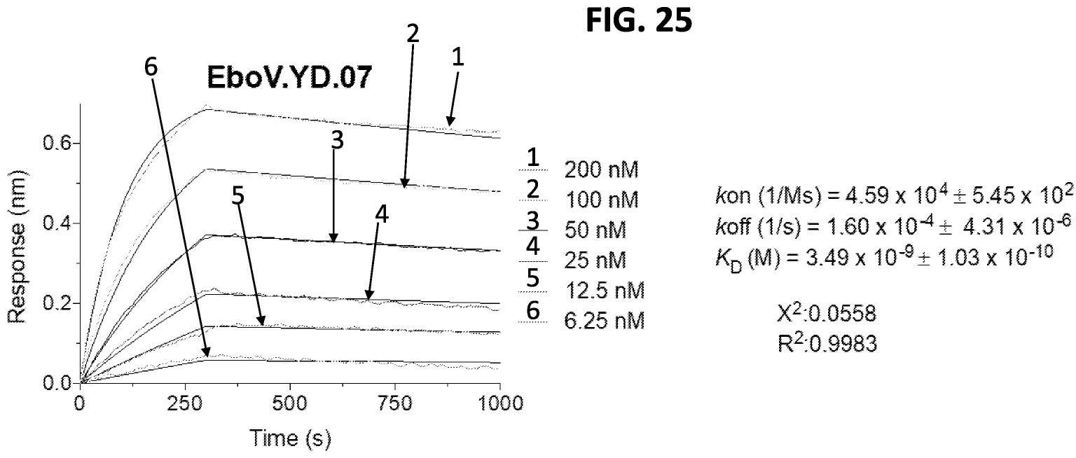

Fab protein generated from EboV.YD.07 IgG was evaluated for binding to EBOV GPΔMuc using BLI. EboV.YD.07 Fab showed binding to EBOV GPΔMuc with an affinity constant (KD) of 3.49 nM, kₒₙ of 4.59×10⁴ per second and k_{off} of 1.60×10⁻⁴ per Molar•second (FIG. 25).

### Ebola Virus mAb EboV.YD.08

The nucleotide and amino acid sequences for the VH and VL domains of the expressed version of EboV.YD.08 are shown below.

EboV.YD.08 VH domain nucleic acid sequence
(SEQ ID NO: 29)
CAGGTCCAGCTGGTGCAGTCTGGGGCTGAGGTGAAGAAGCCTGGGACCTC
AGTGAGGGTCTCCTGCAAGGCTTCTGGATACAGCCTCACCGGCCACTATA
TGCACTGGGTGCGACAGGCCCCTGGACAAGGGCTTGAGTGGATGGGTTGG
ATCAACCCTGCCAGTGGTGGCACACATTATGCAGAGAAGTTTCGGGTCAG
GGTCGCCATGACCAGGGACACGTCCATCAGCACAGTTTACATGGAGTTGT
ACAGCCTGACATCTGACGACACGGCCGTCTACTACTGTGCGAGGGCTGTC
CGGGGCACGACAGCAGTGGCTGGGACTTGGAGGTTCGACCCCTGGGGCCA
GGGAACCCTGGTCATCGTTTCCTCA
EboV.YD.08 VH domain amino acid sequence
(SEQ ID NO: 30)
QVQLVQSGAEVKKPGTSVRVSCKASGYSLTGHYMHWVRQAPGQGLEWMGW
INPASGGTHYAEKFRVRVAMTRDTSISTVYMELYSLTSDDTAVYYCARAV
RGTTAVAGTWRFDPWGQGTLVIVSS
EboV.YD.08 VL domain nucleic acid sequence
(SEQ ID NO: 31)
CAGTCTGTGCTGACTCAGCCGCCCTCAGTGTCTGCGGCCCCAGGACAGAA
GGTCACCATCTCCTGCTCTGGAAGCAGCTCCAACATTGGGAATAATTATG
TATCCTGGTACCAGCAGTTCCCAGGTACAGCCCCCAAACTCCTCATTTAT
GACAATAATAGGCGACCCTCAGGTGTTCCTGACCGATTCTCTGGCTCCAA
GTCTGACACGTCAGCCACCCTGGGCATCACCGGACTCCAGACTGGGGACG
AGGCCGATTATTACTGCGGAACATGGGATAGCAGCCTGGGTGCTGGTGTC
TTCGGCGGAGGGACCAAGCTGACCGTCCTG
EboV.YD.08 VL domain amino acid sequence
(SEQ ID NO: 32)
QSVLTQPPSVSAAPGQKVTISCSGSSSNIGNNYVSWYQQFPGTAPKLLIY
DNNRRPSGVPDRFSGSKSDTSATLGITGLQTGDEADYYCGTWDSSLGAGV
FGGGTKLTVL

| EboV.YD.08 | CDR1 | CDR2 | CDR3 |
|---|---|---|---|
| VH domain (SEQ ID NO: 30) | 26-33 | 51-58 | 96-115 |
| VL domain (SEQ ID NO: 32) | 25-33 | 51-53 | 89-101 |

### Binding of EboV.YD.08 to EBOV GPΔMuc

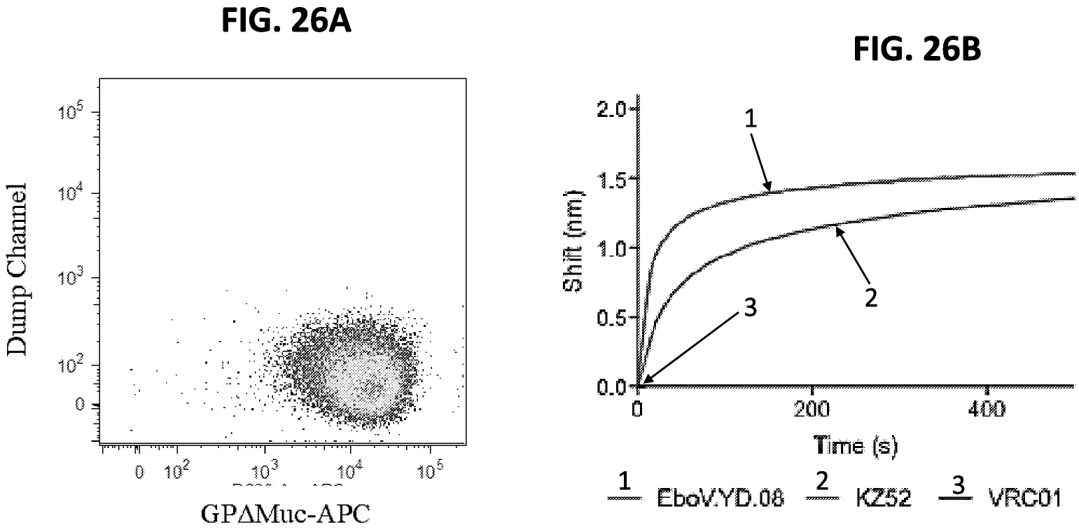

Single colony yeast expressing EboV.YD.08 was derived from sorted gates and reprobed for binding to GPΔMuc using flow cytometry to confirm its expressed Fab bound to GPΔMuc (FIG. 26A). Binding of the full EboV.YD.08 IgG mAbs to GPΔMuc was confirmed using biolayer interferometry (FIG. 26B).

### Epitope Mapping

Gross epitope determination via BLI competition assay was determined as described above (FIG. 11). The assay shows that both KZ52 and S1-4 A09 block binding of EboV.YD.08. This profile suggests that the epitope is near the base of GP (Misasi et al., Science 351(6279): 1343-1346, 2016; Lee et al., Nature 454(7201): 177-182, 2008).

### EboV.YD.08 Kinetics of Binding to EBOV GPΔMuc

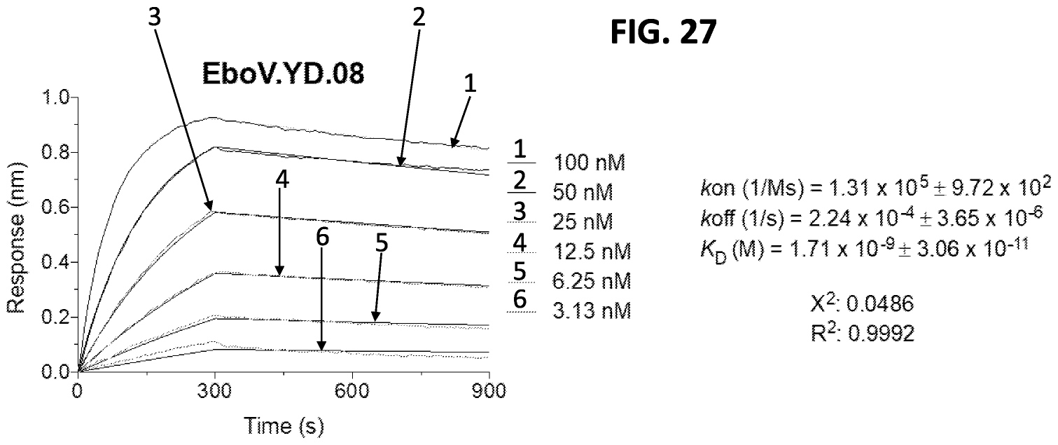

Fab protein generated from EboV.YD.08 IgG was evaluated for binding to EBOV GPΔMuc using BLI. EboV.YD.08 Fab showed binding to EBOV GPΔMuc with an affinity constant (KD) of 1.71 nM, kₒₙ of 1.31×10⁵ per second and k_{off} of 2.24×10⁻⁴ per Molar•second (FIG. 27).

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

1. A monoclonal antibody that specifically binds to Ebola virus (EBOV) glycoprotein (GP), comprising a variable heavy (VH) domain and a variable light (VL) domain, wherein:
(i) the VH domain comprises the VH complementarity determining region (HCDR)1, HCDR2, and HCDR3 sequences of SEQ ID NO: 2 and the VL domain comprises the VL complementarity determining region (LCDR)1, LCDR2, and LCDR3 sequences of SEQ ID NO: 4;
(ii) the VH domain comprises the HCDR1, HCDR2 and HCDR3 sequences of SEQ ID NO: 6 and the VL domain comprises the LCDR1, LCDR2 and LCDR3 sequences of SEQ ID NO: 8;
(iii) the VH domain comprises the HCDR1, HCDR2 and HCDR3 sequences of SEQ ID NO: 10 and the VL domain comprises the LCDR1, LCDR2 and LCDR3 sequences of SEQ ID NO: 12;
(iv) the VH domain comprises the HCDR1, HCDR2 and HCDR3 sequences of SEQ ID NO: 14 and the VL domain comprises the LCDR1, LCDR2 and LCDR3 sequences of SEQ ID NO: 16;
(v) the VH domain comprises the HCDR1, HCDR2 and HCDR3 sequences of SEQ ID NO: 18 and the VL domain comprises the LCDR1, LCDR2 and LCDR3 sequences of SEQ ID NO: 20;
(vi) the VH domain comprises the HCDR1, HCDR2 and HCDR3 sequences of SEQ ID NO: 22 and the VL domain comprises the LCDR1, LCDR2 and LCDR3 sequences of SEQ ID NO: 24;
(vii) the VH domain comprises the HCDR1, HCDR2 and HCDR3 sequences of SEQ ID NO: 26 and the VL domain comprises the LCDR1, LCDR2 and LCDR3 sequences of SEQ ID NO: 28; or
(viii) the VH domain comprises the HCDR1, HCDR2 and HCDR3 sequences of SEQ ID NO: 30 and the VL domain comprises the LCDR1, LCDR2 and LCDR3 sequences of SEQ ID NO: 32.

2. The monoclonal antibody of claim 1, wherein the monoclonal antibody neutralizes Zaire Ebola virus, Sudan Ebola virus, Bundibugyo Ebola virus, or any combination thereof.

3. The monoclonal antibody of claim 2, wherein the neutralization inhibitory concentration 50 (IC50) of the monoclonal antibody is less than 10 μg/ml.

4. The monoclonal antibody of claim 2, wherein the neutralization IC50 of the monoclonal antibody is less than 5 μg/ml.

5. The monoclonal antibody of claim 1, wherein the CDR sequences are determined using the IMGT, Kabat or Chothia numbering scheme.

6. The monoclonal antibody of claim 1, wherein:
the HCDR1, HCDR2, and HCDR3 are respectively set forth as residues 26-33, 51-58 and 96-119 of SEQ ID NO: 2 and the LCDR1, LCDR2, and LCDR3 are respectively set forth as residues 27-37, 50-52 and 88-98 of SEQ ID NO: 4;
the HCDR1, HCDR2, and HCDR3 are respectively set forth as residues 26-33, 51-58 and 96-113 of SEQ ID NO: 6 and the LCDR1, LCDR2, and LCDR3 are respectively set forth as residues 27-32, 50-52 and 89-97 of SEQ ID NO: 8;
the HCDR1, HCDR2, and HCDR3 are respectively set forth as residues 26-33, 51-57 and 95-116 of SEQ ID NO: 10 and the LCDR1, LCDR2, and LCDR3 are respectively set forth as residues 26-31, 49-51 and 87-97 of SEQ ID NO: 12;
the HCDR1, HCDR2, and HCDR3 are respectively set forth as residues 26-33, 51-60 and 98-113 of SEQ ID NO: 14 and the LCDR1, LCDR2, and LCDR3 are respectively set forth as residues 26-34, 52-54 and 90-103 of SEQ ID NO: 16;
the HCDR1, HCDR2, and HCDR3 are respectively set forth as residues 26-33, 51-73 and 111-126 of SEQ ID NO: 18 and the LCDR1, LCDR2, and LCDR3 are respectively set forth as residues 27-32, 50-52 and 88-98 of SEQ ID NO: 20;
the HCDR1, HCDR2, and HCDR3 are respectively set forth as residues 27-35, 53-59 and 97-122 of SEQ ID NO: 22 and the LCDR1, LCDR2, and LCDR3 are respectively set forth as residues 27-32, 50-52 and 88-99 of SEQ ID NO: 24;
the HCDR1, HCDR2, and HCDR3 are respectively set forth as residues 27-33, 51-60 and 98-109 of SEQ ID NO: 26 and the LCDR1, LCDR2, and LCDR3 are respectively set forth as residues 26-34, 52-54 and 90-103 of SEQ ID NO: 28; or
the HCDR1, HCDR2, and HCDR3 are respectively set forth as residues 26-33, 51-58 and 96-115 of SEQ ID NO: 30 and the LCDR1, LCDR2, and LCDR3 are respectively set forth as residues 25-33, 51-53 and 89-101 of SEQ ID NO: 32.

7. The monoclonal antibody of claim 1, wherein:
the amino acid sequence of the VH domain is at least 90% identical to SEQ ID NO: 2 and the amino acid sequence of the VL domain is at least 90% identical to SEQ ID NO: 4;
the amino acid sequence of the VH domain is at least 90% identical to SEQ ID NO: 6 and the amino acid sequence of the VL domain is at least 90% identical to SEQ ID NO: 8;
the amino acid sequence of the VH domain is at least 90% identical to SEQ ID NO: 10 and the amino acid sequence of the VL domain is at least 90% identical to SEQ ID NO: 12;
the amino acid sequence of the VH domain is at least 90% identical to SEQ ID NO: 14 and the amino acid sequence of the VL domain is at least 90% identical to SEQ ID NO: 16;
the amino acid sequence of the VH domain is at least 90% identical to SEQ ID NO: 18 and the amino acid sequence of the VL domain is at least 90% identical to SEQ ID NO: 20;
the amino acid sequence of the VH domain is at least 90% identical to SEQ ID NO: 22 and the amino acid sequence of the VL domain is at least 90% identical to SEQ ID NO: 24;
the amino acid sequence of the VH domain is at least 90% identical to SEQ ID NO: 26 and the amino acid sequence of the VL domain is at least 90% identical to SEQ ID NO: 28; or
the amino acid sequence of the VH domain is at least 90% identical to SEQ ID NO: 30 and the amino acid sequence of the VL domain is at least 90% identical to SEQ ID NO: 32.

8. The monoclonal antibody of wherein:
the amino acid sequence of the VH domain comprises SEQ ID NO: 2 and the amino acid sequence of the VL domain comprises SEQ ID NO: 4;
the amino acid sequence of the VH domain comprises SEQ ID NO: 6 and the amino acid sequence of the VL domain comprises SEQ ID NO: 8;
the amino acid sequence of the VH domain comprises SEQ ID NO: 10 and the amino acid sequence of the VL domain comprises SEQ ID NO: 12;
the amino acid sequence of the VH domain comprises SEQ ID NO: 14 and the amino acid sequence of the VL domain comprises SEQ ID NO: 16;
the amino acid sequence of the VH domain comprises SEQ ID NO: 18 and the amino acid sequence of the VL domain comprises SEQ ID NO: 20;
the amino acid sequence of the VH domain comprises SEQ ID NO: 22 and the amino acid sequence of the VL domain comprises SEQ ID NO: 24;
the amino acid sequence of the VH domain comprises SEQ ID NO: 26 and the amino acid sequence of the VL domain comprises SEQ ID NO: 28; or
the amino acid sequence of the VH domain comprises SEQ ID NO: 30 and the amino acid sequence of the VL domain comprises SEQ ID NO: 32.

9. The monoclonal antibody of wherein the antibody is an IgG, IgM or IgA.

10. The monoclonal antibody of claim 9, wherein the IgG is IgG1.

11. The monoclonal antibody of claim 9, wherein the IgG is IgG2, IgG3 or IgG4.

12. The monoclonal antibody of claim 1, comprising a human constant region.

13. The monoclonal antibody of claim 1, comprising a recombinant constant region comprising a modification that increases binding to the neonatal Fc receptor.

14. The monoclonal antibody of claim 13, wherein the antibody is an IgG1 and the modification that increases binding to the neonatal Fc receptor comprises:
M428L and N434S amino acid substitutions; or
M252Y, S254T and T256E amino acid substitutions.

15. An antigen binding fragment of the monoclonal antibody of claim 1.

16. The antigen-binding fragment of claim 15, wherein the antigen-binding fragment is an Fab fragment, an Fab′ fragment, an F(ab)′2 fragment, a single chain variable fragment (scFv) or a disulfide stabilized variable fragment (dsFv).

17. The monoclonal antibody or antigen binding fragment of claim 1, comprising a human framework region.

18. The monoclonal antibody or antigen-binding fragment of claim 1, which is a fully human antibody or antigen-binding fragment.

19. The monoclonal antibody or antigen binding fragment of claim 1, linked to an effector molecule or a detectable label.

20. (canceled)

21. An isolated nucleic acid molecule encoding the VH domain, the VL domain, or both the VH domain and VL domain of the monoclonal antibody or antigen-binding fragment of claim 1.

22-23. (canceled)

24. The nucleic acid molecule of claim 21, wherein the VH domain and/or the VL domain of the monoclonal antibody or antigen binding fragment comprise the nucleic acid sequences set forth as:
SEQ ID NOs: 1 and 3, respectively, or degenerate variants thereof;
SEQ ID NOs: 5 and 7, respectively, or degenerate variants thereof;
SEQ ID NOs: 9 and 11, respectively, or degenerate variants thereof;
SEQ ID NOs: 13 and 15, respectively, or degenerate variants thereof;
SEQ ID NOs: 17 and 19, respectively, or degenerate variants thereof;
SEQ ID NOs: 21 and 23, respectively, or degenerate variants thereof;
SEQ ID NOs: 25 and 27, respectively, or degenerate variants thereof;
SEQ ID NOs: 29 and 31, respectively, or degenerate variants thereof.

25. The nucleic acid molecule of claim 21, operably linked to a promoter.

26. An expression vector comprising the nucleic acid molecule of claim 25.

27. A pharmaceutical composition for use in treating or inhibiting an Ebola virus infection, comprising:
a therapeutically effective amount of the monoclonal antibody, antigen binding fragment, nucleic acid molecule, or expression vector of claim 1; and
a pharmaceutically acceptable carrier.

28. The pharmaceutical composition of claim 27, wherein the composition is sterile and/or is in unit dosage form or a multiple thereof.

29. A method of detecting an Ebola virus infection in a subject, comprising:
contacting a biological sample from the subject with the monoclonal antibody or antigen binding fragment of claim 1 under conditions sufficient to form an immune complex; and
detecting the presence of the immune complex in the sample, wherein the presence of the immune complex in the sample indicates that the subject has the Ebola virus infection.

30. A method of preventing or treating an Ebola virus infection in a subject, comprising administering to the subject a therapeutically effective amount of the antibody of claim 1, thereby preventing or treating the Ebola virus infection.

31. The method of claim 30, further comprising administering to the subject one or more additional antibodies or antigen binding fragments that specifically bind to Ebola virus GP and neutralize Ebola virus, or one or more nucleic acid molecules encoding the additional antibodies or antigen binding fragments.

32. The method of claim 30, wherein the Ebola virus is Ebola virus Zaire.

33. A method of producing a monoclonal antibody or antigen binding fragment that specifically binds to Ebola virus GP, comprising:
expressing first and second nucleic acid molecules encoding the VH domain and the VL domain, respectively, of the monoclonal antibody of claim 1 in a host cell, or expressing a nucleic acid molecule encoding the VH domain and the VL domain of the monoclonal antibody in the host cell; and
purifying the antibody or antigen binding fragment;
thereby producing the antibody or antigen binding fragment.

34. (canceled)
